SEQUENCE LISTING (287 sequences)

SEQ ID NO: 1 - RNA, 20 nt, SARS-CoV-2
  source 1..20: mol_type = other RNA
tgattgtgaagaagaagagt

SEQ ID NO: 2 - DNA, 20 bp, SARS-CoV-2
  source 1..20: mol_type = other DNA
tgattgtgaagaagaagagt

SEQ ID NO: 3 - RNA, 20 nt, SARS-CoV-2
  source 1..20: mol_type = other RNA
aagaaaggagctaaattgtt

SEQ ID NO: 4 - DNA, 20 bp, SARS-CoV-2
  source 1..20: mol_type = other DNA
aagaaaggagctaaattgtt

SEQ ID NO: 5 - RNA, 20 nt, SARS-CoV-2
  source 1..20: mol_type = other RNA
agagttgatttttgtggaaa

SEQ ID NO: 6 - DNA, 20 bp, SARS-CoV-2
  source 1..20: mol_type = other DNA
agagttgatttttgtggaaa

SEQ ID NO: 7 - RNA, 20 nt, SARS-CoV-2
  source 1..20: mol_type = other RNA
tggtgtttattctgttattt

SEQ ID NO: 8 - DNA, 20 bp, SARS-CoV-2
  source 1..20: mol_type = other DNA
tggtgtttattctgttattt

SEQ ID NO: 9 - RNA, 20 nt, SARS-CoV-2
  source 1..20: mol_type = other RNA
ggtaaagttgagggttgtat

SEQ ID NO: 10 - DNA, 20 bp, SARS-CoV-2
  source 1..20: mol_type = other DNA
ggtaaagttgagggttgtat

SEQ ID NO: 11 - DNA, 20 bp, Escherichia virus MS2
  source 1..20: mol_type = other DNA
accactaatgagtgatatcc

SEQ ID NO: 12 - DNA, 20 bp, Escherichia virus MS2
  source 1..20: mol_type = other DNA
tacctgtaggtaacatgctc

SEQ ID NO: 13 - DNA, 20 bp, Escherichia virus MS2
  source 1..20: mol_type = other DNA
tctgcatccgattccatctc

SEQ ID NO: 14 - DNA, 20 bp, Escherichia virus MS2
  source 1..20: mol_type = other DNA
cctgatatgaatatgtacct

SEQ ID NO: 15 - DNA, 20 bp, Escherichia virus MS2
  source 1..20: mol_type = other DNA
acctccccctaaagagagga

SEQ ID NO: 16 - DNA, 20 bp, synthetic construct
  source 1..20: mol_type = other DNA
aaccaaccgaactgcaactc

SEQ ID NO: 17 - DNA, 20 bp, synthetic construct
  source 1..20: mol_type = other DNA
aagcatctcatatgcaccct

SEQ ID NO: 18 - DNA, 20 bp, synthetic construct
  source 1..20: mol_type = other DNA
ggagccagtcgacaacgaat

SEQ ID NO: 19 - DNA, 20 bp, synthetic construct
  source 1..20: mol_type = other DNA
acgggggccgtaaggccctc

SEQ ID NO: 20 - DNA, 20 bp, synthetic construct
  source 1..20: mol_type = other DNA
cgataagtctatcgtcgcaa

SEQ ID NO: 21 - DNA, 20 bp, synthetic construct
  source 1..20: mol_type = other DNA
aactccacaccaggcgatcg

SEQ ID NO: 22 - DNA, 63 bp, synthetic construct
  source 1..63: mol_type = other DNA
ttcgacaactcgtattaaatcctttgcccgaacgttattttttactcttcttcttcacaatca

SEQ ID NO: 23 - DNA, 63 bp, synthetic construct
  source 1..63: mol_type = other DNA
gtgagtgaataaccttgcttctgtaaatcgtcgctatttttttaacaatttagctcctttctt

SEQ ID NO: 24 - DNA, 63 bp, synthetic construct
  source 1..63: mol_type = other DNA
agaatataaagtaccgacaaaaggtaaagtaattctgtttttttttccacaaaaatcaactct

SEQ ID NO: 25 - DNA, 63 bp, synthetic construct
  source 1..63: mol_type = other DNA
tcccaatccaaataagaaacgattttttgtttaacgtctttttaaataacagaataaacacca

SEQ ID NO: 26 - DNA, 63 bp, synthetic construct
  source 1..63: mol_type = other DNA
cattcaaccgattgagggagggaaggtaaatattgacgtttttatacaaccctcaactttacc

SEQ ID NO: 27 - DNA, 63 bp, synthetic construct
  source 1..63: mol_type = other DNA
ttcgacaactcgtattaaatcctttgcccgaacgttattttttggatatcactcattagtggt

SEQ ID NO: 28 - DNA, 63 bp, synthetic construct
  source 1..63: mol_type = other DNA
gtgagtgaataaccttgcttctgtaaatcgtcgctatttttttgagcatgttacctacaggta

SEQ ID NO: 29 - DNA, 63 bp, synthetic construct
  source 1..63: mol_type = other DNA
agaatataaagtaccgacaaaaggtaaagtaattctgttttttgagatggaatcggatgcaga

SEQ ID NO: 30 - DNA, 358 bp, synthetic construct
  source 1..358: mol_type = other DNA
acatcacttgcctgagtagaagaactcaaatcctcttttgaggaacaagttttcttgtctatcggccttgctggtaattcctcttttgaggaacaagttttcttgtatccagaacaatattaccgctcctcttttgaggaacaagttttcttgtcagccattgcaacaggaaaatcctcttttgaggaacaagttttcttgtacgctcatggaaatacctactcctcttttgaggaacaagttttcttgtattttgacgctcaatcgtcttcctcttttgaggaacaagttttcttgtgaaatggattatttacattggcagattcaccagtcacacgaccagtaataaaagggacat

SEQ ID NO: 31 - DNA, 434 bp, synthetic construct
  source 1..434: mol_type = other DNA
tcacaaacaaataaatcctcattaaagccagaatggaaagcgcagtctctgaatttaccgttccagtaagcgtcatacatggcttttcctcttttgaggaacaagttttcttgttgatgatacaggagtgtacttcctcttttgaggaacaagttttcttgtggtaataagttttaacggggtcctcttttgaggaacaagttttcttgttcagtgccttgagtaacagttcctcttttgaggaacaagttttcttgtgcccgtataaacagttaatgtcctcttttgaggaacaagttttcttgtccccctgcctatttcggaactcctcttttgaggaacaagttttcttgtctattattctgaaacatgaaagtattaagaggctgagactcctcaagagaaggattaggattagcggggttttgctcagt

SEQ ID NO: 32 - DNA, 14 bp, synthetic construct
  source 1..14: mol_type = other DNA
tgaagaagaagagt

SEQ ID NO: 33 - DNA, 14 bp, synthetic construct
  source 1..14: mol_type = other DNA
ggagctaaattgtt

SEQ ID NO: 34 - DNA, 14 bp, synthetic construct
  source 1..14: mol_type = other DNA
gatttttgtggaaa

SEQ ID NO: 35 - DNA, 14 bp, synthetic construct
  source 1..14: mol_type = other DNA
ttattctgttattt

SEQ ID NO: 36 - DNA, 14 bp, synthetic construct
  source 1..14: mol_type = other DNA
gttgagggttgtat

SEQ ID NO: 37 - DNA, 14 bp, synthetic construct
  source 1..14: mol_type = other DNA
aatgagtgatatcc

SEQ ID NO: 38 - DNA, 14 bp, synthetic construct
  source 1..14: mol_type = other DNA
taggtaacatgctc

SEQ ID NO: 39 - DNA, 14 bp, synthetic construct
  source 1..14: mol_type = other DNA
tccgattccatctc

SEQ ID NO: 40 - DNA, 20 bp, synthetic construct
  source 1..20: mol_type = other DNA
gctagtgtaactagcaagaa

SEQ ID NO: 41 - DNA, 20 bp, synthetic construct
  source 1..20: mol_type = other DNA
attgcagcagtacgcacaca

SEQ ID NO: 42 - DNA, 20 bp, synthetic construct
  source 1..20: mol_type = other DNA
cgttctccattctggttact

SEQ ID NO: 43 - DNA, 20 bp, synthetic construct
  source 1..20: mol_type = other DNA
gtgatcttttggtgtattca

SEQ ID NO: 44 - DNA, 20 bp, synthetic construct
  source 1..20: mol_type = other DNA
gataactagcgcatatacct

SEQ ID NO: 45 - DNA, 20 bp, synthetic construct
  source 1..20: mol_type = other DNA
gctacactacgtgcccgccg

SEQ ID NO: 46 - DNA, 20 bp, SARS-CoV-2
  source 1..20: mol_type = other DNA
catccttactgcgcttcgat

SEQ ID NO: 47 - DNA, 20 bp, SARS-CoV-2
  source 1..20: mol_type = other DNA
cattgcaactgagggagcct

SEQ ID NO: 48 - DNA, 20 bp, SARS-CoV-2
  source 1..20: mol_type = other DNA
agactcagactaattctcct

SEQ ID NO: 49 - DNA, 14 bp, synthetic construct
  source 1..14: mol_type = other DNA
tactgcgcttcgat

SEQ ID NO: 50 - DNA, 14 bp, synthetic construct
  source 1..14: mol_type = other DNA
aactgagggagcct

SEQ ID NO: 51 - DNA, 14 bp, synthetic construct
  source 1..14: mol_type = other DNA
agactaattctcct

SEQ ID NO: 52 - DNA, 63 bp, synthetic construct
  source 1..63: mol_type = other DNA
ttcgacaactcgtattaaatcctttgcccgaacgttattttttatcgaagcgcagtaaggatg

SEQ ID NO: 53 - DNA, 63 bp, synthetic construct
  source 1..63: mol_type = other DNA
gtgagtgaataaccttgcttctgtaaatcgtcgctatttttttaggctccctcagttgcaatg

SEQ ID NO: 54 - DNA, 63 bp, synthetic construct
  source 1..63: mol_type = other DNA
agaatataaagtaccgacaaaaggtaaagtaattctgttttttaggagaattagtctgagtct

SEQ ID NO: 55 - DNA, 20 bp, SARS-CoV-2
  source 1..20: mol_type = other DNA
gtatgaaaatgcctttttac

SEQ ID NO: 56 - DNA, 20 bp, Influenza A virus
  source 1..20: mol_type = other DNA
tgacaggattggtcttgtct

SEQ ID NO: 57 - DNA, 20 bp, Respiratory syncytial virus universal A
  source 1..20: mol_type = other DNA
acacagcagctgttcagtac

SEQ ID NO: 58 - DNA, 20 bp, Parainfluenza 1
  source 1..20: mol_type = other DNA
cttcctgctggtgtgttaat

SEQ ID NO: 59 - DNA, 20 bp, Rhinovirus
  source 1..20: mol_type = other DNA
tcctccggcccctgaatgtg

SEQ ID NO: 60 - DNA, 14 bp, synthetic construct
  source 1..14: mol_type = other DNA
aaatgcctttttac

SEQ ID NO: 61 - DNA, 14 bp, synthetic construct
  source 1..14: mol_type = other DNA
gattggtcttgtct

SEQ ID NO: 62 - DNA, 14 bp, synthetic construct
  source 1..14: mol_type = other DNA
cagctgttcagtac

SEQ ID NO: 63 - DNA, 14 bp, synthetic construct
  source 1..14: mol_type = other DNA
gctggtgtgttaat

SEQ ID NO: 64 - DNA, 14 bp, synthetic construct
  source 1..14: mol_type = other DNA
ggcccctgaatgtg

SEQ ID NO: 65 - DNA, 63 bp, synthetic construct
  source 1..63: mol_type = other DNA
ttcgacaactcgtattaaatcctttgcccgaacgttattttttgtaaaaaggcattttcatac

SEQ ID NO: 66 - DNA, 63 bp, synthetic construct
  source 1..63: mol_type = other DNA
gtgagtgaataaccttgcttctgtaaatcgtcgctatttttttgtactgaacagctgctgtgt

SEQ ID NO: 67 - DNA, 63 bp, synthetic construct
  source 1..63: mol_type = other DNA
agaatataaagtaccgacaaaaggtaaagtaattctgttttttcacattcaggggccggagga

SEQ ID NO: 68 - DNA, 63 bp, synthetic construct
  source 1..63: mol_type = other DNA
tcccaatccaaataagaaacgattttttgtttaacgtctttttagacaagaccaatcctgtca

SEQ ID NO: 69 - DNA, 63 bp, synthetic construct
  source 1..63: mol_type = other DNA
cattcaaccgattgagggagggaaggtaaatattgacgtttttattaacacaccagcaggaag

SEQ ID NO: 70 - DNA, 20 bp, SARS-CoV-2
  source 1..20: mol_type = other DNA
gtatgaaaatgcctttttac

SEQ ID NO: 71 - DNA, 20 bp, SARS-CoV-2
  source 1..20: mol_type = other DNA
ccggtatagattgtttagga

SEQ ID NO: 72 - DNA, 20 bp, SARS-CoV-2
  source 1..20: mol_type = other DNA
ggtgttaactgcacagaagt

SEQ ID NO: 73 - DNA, 20 bp, SARS-CoV-2
  source 1..20: mol_type = other DNA
cttatggtgttggttaccaa

SEQ ID NO: 74 - DNA, 20 bp, SARS-CoV-2
  source 1..20: mol_type = other DNA
taaaggttttaattgttact

SEQ ID NO: 75 - DNA, 14 bp, synthetic construct
  source 1..14: mol_type = other DNA
aaatgcctttttac

SEQ ID NO: 76 - DNA, 14 bp, synthetic construct
  source 1..14: mol_type = other DNA
tagattgtttagga

SEQ ID NO: 77 - DNA, 14 bp, synthetic construct
  source 1..14: mol_type = other DNA
aactgcacagaagt

SEQ ID NO: 78 - DNA, 14 bp, synthetic construct
  source 1..14: mol_type = other DNA
gtgttggttaccaa

SEQ ID NO: 79 - DNA, 14 bp, synthetic construct
  source 1..14: mol_type = other DNA
ttttaattgttact

SEQ ID NO: 80 - DNA, 20 bp, SARS-CoV-2
  source 1..20: mol_type = other DNA
cctgtatagattgtttagga

SEQ ID NO: 81 - DNA, 20 bp, SARS-CoV-2
  source 1..20: mol_type = other DNA
gatgttaactgcacagaagt

SEQ ID NO: 82 - DNA, 20 bp, SARS-CoV-2
  source 1..20: mol_type = other DNA
ctaatggtgttggttaccaa

SEQ ID NO: 83 - DNA, 20 bp, SARS-CoV-2
  source 1..20: mol_type = other DNA
tgaaggttttaattgttact

SEQ ID NO: 84 - DNA, 63 bp, synthetic construct; identical to SEQ ID NO: 65
  source 1..63: mol_type = other DNA

SEQ ID NO: 85 - DNA, 63 bp, synthetic construct
  source 1..63: mol_type = other DNA
agaatataaagtaccgacaaaaggtaaagtaattctgtttttttcctaaacaatctataccgg

SEQ ID NO: 86 - DNA, 63 bp, synthetic construct
  source 1..63: mol_type = other DNA
cattcaaccgattgagggagggaaggtaaatattgacgtttttacttctgtgcagttaacacc

SEQ ID NO: 87 - DNA, 63 bp, synthetic construct
  source 1..63: mol_type = other DNA
tcccaatccaaataagaaacgattttttgtttaacgtctttttttggtaaccaacaccataag

SEQ ID NO: 88 - DNA, 63 bp, synthetic construct
  source 1..63: mol_type = other DNA
gtgagtgaataaccttgcttctgtaaatcgtcgctatttttttagtaacaattaaaaccttta

SEQ ID NO: 89 - DNA, 46 bp, synthetic construct
  source 1..46: mol_type = other DNA
ttttcgtaatcatggtcatagctgtttcctgtgtgaaattgttatc

SEQ ID NO: 90 - DNA, 38 bp, synthetic construct
  source 1..38: mol_type = other DNA
cgctcacaattccacacaacatacgagccggaagcata

SEQ ID NO: 91 - DNA, 38 bp, synthetic construct
  source 1..38: mol_type = other DNA
aagtgtaaagcctggggtgcctaatgagtgagctaact

SEQ ID NO: 92 - DNA, 38 bp, synthetic construct
  source 1..38: mol_type = other DNA
cacattaattgcgttgcgctcactgcccgctttccagt

SEQ ID NO: 93 - DNA, 38 bp, synthetic construct
  source 1..38: mol_type = other DNA
cgggaaacctgtcgtgccagctgcattaatgaatcggc

SEQ ID NO: 94 - DNA, 38 bp, synthetic construct
  source 1..38: mol_type = other DNA
caacgcgcggggagaggcggtttgcgtattgggcgcca

SEQ ID NO: 95 - DNA, 38 bp, synthetic construct
  source 1..38: mol_type = other DNA
gggtggtttttcttttcaccagtgagacgggcaacagc

SEQ ID NO: 96 - DNA, 38 bp, synthetic construct
  source 1..38: mol_type = other DNA
tgattgcccttcaccgcctggccctgagagagttgcag

SEQ ID NO: 97 - DNA, 38 bp, synthetic construct
  source 1..38: mol_type = other DNA
caagcggtccacgctggtttgccccagcaggcgaaaat

SEQ ID NO: 98 - DNA, 38 bp, synthetic construct
  source 1..38: mol_type = other DNA
cctgtttgatggtggttccgaaatcggcaaaatccctt

SEQ ID NO: 99 - DNA, 38 bp, synthetic construct
  source 1..38: mol_type = other DNA
ataaatcaaaagaatagcccgagatagggttgagtgtt

SEQ ID NO: 100 - DNA, 38 bp, synthetic construct
  source 1..38: mol_type = other DNA
gttccagtttggaacaagagtccactattaaagaacgt

SEQ ID NO: 101 - DNA, 38 bp, synthetic construct
  source 1..38: mol_type = other DNA
ggactccaacgtcaaagggcgaaaaaccgtctatcagg

SEQ ID NO: 102 - DNA, 38 bp, synthetic construct
  source 1..38: mol_type = other DNA
gcgatggcccactacgtgaaccatcacccaaatcaagt

SEQ ID NO: 103 - DNA, 38 bp, synthetic construct
  source 1..38: mol_type = other DNA
tttttggggtcgaggtgccgtaaagcactaaatcggaa

SEQ ID NO: 104 - DNA, 38 bp, synthetic construct
  source 1..38: mol_type = other DNA
ccctaaagggagcccccgatttagagcttgacggggaa

SEQ ID NO: 105 - DNA, 38 bp, synthetic construct
  source 1..38: mol_type = other DNA
agccggcgaacgtggcgagaaaggaagggaagaaagcg

SEQ ID NO: 106 - DNA, 38 bp, synthetic construct
  source 1..38: mol_type = other DNA
aaaggagcgggcgctagggcgctggcaagtgtagcggt

SEQ ID NO: 107 - DNA, 38 bp, synthetic construct
  source 1..38: mol_type = other DNA
cacgctgcgcgtaaccaccacacccgccgcgcttaatg

SEQ ID NO: 108 - DNA, 38 bp, synthetic construct
  source 1..38: mol_type = other DNA
cgccgctacagggcgcgtactatggttgctttgacgag

SEQ ID NO: 109 - DNA, 38 bp, synthetic construct
  source 1..38: mol_type = other DNA
cacgtataacgtgctttcctcgttagaatcagagcggg

SEQ ID NO: 110 - DNA, 38 bp, synthetic construct
  source 1..38: mol_type = other DNA
agctaaacaggaggccgattaaagggattttagacagg

SEQ ID NO: 111 - DNA, 38 bp, synthetic construct
  source 1..38: mol_type = other DNA
aacggtacgccagaatcctgagaagtgtttttataatc

SEQ ID NO: 112 - DNA, 38 bp, synthetic construct
  source 1..38: mol_type = other DNA
agtgaggccaccgagtaaaagagtctgtccatcacgca

SEQ ID NO: 113 - DNA, 38 bp, synthetic construct
  source 1..38: mol_type = other DNA
aattaaccgttgtagcaatacttctttgattagtaata

SEQ ID NO: 114 - DNA, 38 bp, synthetic construct
  source 1..38: mol_type = other DNA
acatcacttgcctgagtagaagaactcaaactatcggc

SEQ ID NO: 115 - DNA, 38 bp, synthetic construct
  source 1..38: mol_type = other DNA
cttgctggtaatatccagaacaatattaccgccagcca

SEQ ID NO: 116 - DNA, 38 bp, synthetic construct
  source 1..38: mol_type = other DNA
ttgcaacaggaaaaacgctcatggaaatacctacattt

SEQ ID NO: 117 - DNA, 38 bp, synthetic construct
  source 1..38: mol_type = other DNA
tgacgctcaatcgtctgaaatggattatttacattggc

SEQ ID NO: 118 - DNA, 38 bp, synthetic construct
  source 1..38: mol_type = other DNA
agattcaccagtcacacgaccagtaataaaagggacat

SEQ ID NO: 119 - DNA, 38 bp, synthetic construct
  source 1..38: mol_type = other DNA
tctggccaacagagatagaacccttctgacctgaaagc

SEQ ID NO: 120 - DNA, 38 bp, synthetic construct
  source 1..38: mol_type = other DNA
gtaagaatacgtggcacagacaatatttttgaatggct

SEQ ID NO: 121 - DNA, 38 bp, synthetic construct
  source 1..38: mol_type = other DNA
attagtctttaatgcgcgaactgatagccctaaaacat

SEQ ID NO: 122 - DNA, 38 bp, synthetic construct
  source 1..38: mol_type = other DNA
cgccattaaaaataccgaacgaaccaccagcagaagat

SEQ ID NO: 123 - DNA, 38 bp, synthetic construct
  source 1..38: mol_type = other DNA
aaaacagaggtgaggcggtcagtattaacaccgcctgc

SEQ ID NO: 124 - DNA, 38 bp, synthetic construct
  source 1..38: mol_type = other DNA
aacagtgccacgctgagagccagcagcaaatgaaaaat

SEQ ID NO: 125 - DNA, 38 bp, synthetic construct
  source 1..38: mol_type = other DNA
ctaaagcatcaccttgctgaacctcaaatatcaaaccc

SEQ ID NO: 126 - DNA, 38 bp, synthetic construct
  source 1..38: mol_type = other DNA
tcaatcaatatctggtcagttggcaaatcaacagttga

SEQ ID NO: 127 - DNA, 38 bp, synthetic construct
  source 1..38: mol_type = other DNA
aaggaattgaggaaggttatctaaaatatctttaggag

SEQ ID NO: 128 - DNA, 38 bp, synthetic construct
  source 1..38: mol_type = other DNA
cactaacaactaatagattagagccgtcaatagataat

SEQ ID NO: 129 - DNA, 38 bp, synthetic construct
  source 1..38: mol_type = other DNA
acatttgaggatttagaagtattagactttacaaacaa

SEQ ID NO: 130 - DNA, 38 bp, synthetic construct
  source 1..38: mol_type = other DNA
ttcgacaactcgtattaaatcctttgcccgaacgttat

SEQ ID NO: 131 - DNA, 38 bp, synthetic construct
  source 1..38: mol_type = other DNA
taattttaaaagtttgagtaacattatcattttgcgga

SEQ ID NO: 132 - DNA, 38 bp, synthetic construct
  source 1..38: mol_type = other DNA
acaaagaaaccaccagaaggagcggaattatcatcata

SEQ ID NO: 133 - DNA, 38 bp, synthetic construct
  source 1..38: mol_type = other DNA
ttcctgattatcagatgatggcaattcatcaatataat

SEQ ID NO: 134 - DNA, 38 bp, synthetic construct
  source 1..38: mol_type = other DNA
cctgattgtttggattatacttctgaataatggaaggg

SEQ ID NO: 135 - DNA, 38 bp, synthetic construct
  source 1..38: mol_type = other DNA
ttagaacctaccatatcaaaattatttgcacgtaaaac

SEQ ID NO: 136 - DNA, 38 bp, synthetic construct
  source 1..38: mol_type = other DNA
agaaataaagaaattgcgtagattttcaggtttaacgt

SEQ ID NO: 137 - DNA, 38 bp, synthetic construct
  source 1..38: mol_type = other DNA
cagatgaatatacagtaacagtaccttttacatcggga

SEQ ID NO: 138 - DNA, 38 bp, synthetic construct
  source 1..38: mol_type = other DNA
gaaacaataacggattcgcctgattgctttgaatacca

SEQ ID NO: 139 - DNA, 38 bp, synthetic construct
  source 1..38: mol_type = other DNA
agttacaaaatcgcgcagaggcgaattattcatttcaa

SEQ ID NO: 140 - DNA, 38 bp, synthetic construct
  source 1..38: mol_type = other DNA
ttacctgagcaaaagaagatgatgaaacaaacatcaag

SEQ ID NO: 141 - DNA, 38 bp, synthetic construct
  source 1..38: mol_type = other DNA
aaaacaaaattaattacatttaacaatttcatttgaat

SEQ ID NO: 142 - DNA, 38 bp, synthetic construct
  source 1..38: mol_type = other DNA
taccttttttaatggaaacagtacataaatcaatatat

SEQ ID NO: 143 - DNA, 38 bp, synthetic construct
  source 1..38: mol_type = other DNA
gtgagtgaataaccttgcttctgtaaatcgtcgctatt

SEQ ID NO: 144 - DNA, 38 bp, synthetic construct
  source 1..38: mol_type = other DNA
aattaattttcccttagaatccttgaaaacatagcgat

SEQ ID NO: 145 - DNA, 38 bp, synthetic construct
  source 1..38: mol_type = other DNA
agcttagattaagacgctgagaagagtcaatagtgaat

SEQ ID NO: 146 - DNA, 38 bp, synthetic construct
  source 1..38: mol_type = other DNA
ttatcaaaatcataggtctgagagactacctttttaac

SEQ ID NO: 147 - DNA, 38 bp, synthetic construct
  source 1..38: mol_type = other DNA
ctccggcttaggttgggttatataactatatgtaaatg

SEQ ID NO: 148 - DNA, 38 bp, synthetic construct
  source 1..38: mol_type = other DNA
ctgatgcaaatccaatcgcaagacaaagaacgcgagaa

SEQ ID NO: 149 - DNA, 38 bp, synthetic construct
  source 1..38: mol_type = other DNA
aactttttcaaatatattttagttaatttcatcttctg

SEQ ID NO: 150 - DNA, 38 bp, synthetic construct
  source 1..38: mol_type = other DNA
acctaaatttaatggtttgaaataccgaccgtgtgata

SEQ ID NO: 151 - DNA, 38 bp, synthetic construct
  source 1..38: mol_type = other DNA
aataaggcgttaaataagaataaacaccggaatcataa

SEQ ID NO: 152 - DNA, 38 bp, synthetic construct
  source 1..38: mol_type = other DNA
ttactagaaaaagcctgtttagtatcatatgcgttata

SEQ ID NO: 153 - DNA, 38 bp, synthetic construct
  source 1..38: mol_type = other DNA
caaattcttaccagtataaagccaacgctcaacagtag

SEQ ID NO: 154 - DNA, 38 bp, synthetic construct
  source 1..38: mol_type = other DNA
ggcttaattgagaatcgccatatttaacaacgccaaca

SEQ ID NO: 155 - DNA, 38 bp, synthetic construct
  source 1..38: mol_type = other DNA
tgtaatttaggcagaggcattttcgagccagtaataag

SEQ ID NO: 156 - DNA, 38 bp, synthetic construct
  source 1..38: mol_type = other DNA
agaatataaagtaccgacaaaaggtaaagtaattctgt

SEQ ID NO: 157 - DNA, 38 bp, synthetic construct
  source 1..38: mol_type = other DNA
ccagacgacgacaataaacaacatgttcagctaatgca

SEQ ID NO: 158 - DNA, 38 bp, synthetic construct
  source 1..38: mol_type = other DNA
gaacgcgcctgtttatcaacaatagataagtcctgaac

SEQ ID NO: 159 - DNA, 38 bp, synthetic construct
  source 1..38: mol_type = other DNA
aagaaaaataatatcccatcctaatttacgagcatgta

SEQ ID NO: 160 - DNA, 38 bp, synthetic construct
  source 1..38: mol_type = other DNA
gaaaccaatcaataatcggctgtctttccttatcattc

SEQ ID NO: 161 - DNA, 38 bp, synthetic construct
  source 1..38: mol_type = other DNA
caagaacgggtattaaaccaagtaccgcactcatcgag

SEQ ID NO: 162 - DNA, 38 bp, synthetic construct
  source 1..38: mol_type = other DNA
aacaagcaagccgtttttattttcatcgtaggaatcat

SEQ ID NO: 163 - DNA, 38 bp, synthetic construct
  source 1..38: mol_type = other DNA
taccgcgcccaatagcaagcaaatcagatatagaaggc

SEQ ID NO: 164 - DNA, 38 bp, synthetic construct
  source 1..38: mol_type = other DNA
ttatccggtattctaagaacgcgaggcgttttagcgaa

SEQ ID NO: 165 - DNA, 38 bp, synthetic construct
  source 1..38: mol_type = other DNA
cctcccgacttgcgggaggttttgaagccttaaatcaa

SEQ ID NO: 166 - DNA, 38 bp, synthetic construct
  source 1..38: mol_type = other DNA
gattagttgctattttgcacccagctacaattttatcc

SEQ ID NO: 167 - DNA, 38 bp, synthetic construct
  source 1..38: mol_type = other DNA
tgaatcttaccaacgctaacgagcgtctttccagagcc

SEQ ID NO: 168 - DNA, 38 bp, synthetic construct
  source 1..38: mol_type = other DNA
taatttgccagttacaaaataaacagccatattattta

SEQ ID NO: 169 - DNA, 38 bp, synthetic construct
  source 1..38: mol_type = other DNA
tcccaatccaaataagaaacgattttttgtttaacgtc

SEQ ID NO: 170 - DNA, 38 bp, synthetic construct
  source 1..38: mol_type = other DNA
aaaaatgaaaatagcagcctttacagagagaataacat

SEQ ID NO: 171 - DNA, 38 bp, synthetic construct
  source 1..38: mol_type = other DNA
aaaaacagggaagcgcattagacgggagaattaactga

SEQ ID NO: 172 - DNA, 38 bp, synthetic construct
  source 1..38: mol_type = other DNA
acaccctgaacaaagtcagagggtaattgagcgctaat

SEQ ID NO: 173 - DNA, 38 bp, synthetic construct
  source 1..38: mol_type = other DNA
atcagagagataacccacaagaattgagttaagcccaa

SEQ ID NO: 174 - DNA, 38 bp, synthetic construct
  source 1..38: mol_type = other DNA
taataagagcaagaaacaatgaaatagcaatagctatc

SEQ ID NO: 175 - DNA, 38 bp, synthetic construct
  source 1..38: mol_type = other DNA
ttaccgaagccctttttaagaaaagtaagcagatagcc

SEQ ID NO: 176 - DNA, 38 bp, synthetic construct
  source 1..38: mol_type = other DNA
gaacaaagttaccagaaggaaaccgaggaaacgcaata

SEQ ID NO: 177 - DNA, 38 bp, synthetic construct
  source 1..38: mol_type = other DNA
ataacggaatacccaaaagaactggcatgattaagact

SEQ ID NO: 178 - DNA, 38 bp, synthetic construct
  source 1..38: mol_type = other DNA
ccttattacgcagtatgttagcaaacgtagaaaataca

SEQ ID NO: 179 - DNA, 38 bp, synthetic construct
  source 1..38: mol_type = other DNA
tacataaaggtggcaacatataaaagaaacgcaaagac

SEQ ID NO: 180 - DNA, 38 bp, synthetic construct
  source 1..38: mol_type = other DNA
accacggaataagtttattttgtcacaatcaatagaaa

SEQ ID NO: 181 - DNA, 38 bp, synthetic construct
  source 1..38: mol_type = other DNA
attcatatggtttaccagcgccaaagacaaaagggcga

SEQ ID NO: 182 - DNA, 38 bp, synthetic construct
  source 1..38: mol_type = other DNA
cattcaaccgattgagggagggaaggtaaatattgacg

SEQ ID NO: 183 - DNA, 38 bp, synthetic construct
  source 1..38: mol_type = other DNA
gaaattattcattaaaggtgaattatcaccgtcaccga

SEQ ID NO: 184 - DNA, 38 bp, synthetic construct
  source 1..38: mol_type = other DNA
cttgagccatttgggaattagagccagcaaaatcacca

SEQ ID NO: 185 - DNA, 38 bp, synthetic construct
  source 1..38: mol_type = other DNA
gtagcaccattaccattagcaaggccggaaacgtcacc

SEQ ID NO: 186 - DNA, 38 bp, synthetic construct
  source 1..38: mol_type = other DNA
aatgaaaccatcgatagcagcaccgtaatcagtagcga

SEQ ID NO: 187 - DNA, 38 bp, synthetic construct
  source 1..38: mol_type = other DNA
cagaatcaagtttgcctttagcgtcagactgtagcgcg

SEQ ID NO: 188 - DNA, 38 bp, synthetic construct
  source 1..38: mol_type = other DNA
ttttcatcggcattttcggtcatagcccccttattagc

SEQ ID NO: 189 - DNA, 38 bp, synthetic construct
  source 1..38: mol_type = other DNA
gtttgccatcttttcataatcaaaatcaccggaaccag

SEQ ID NO: 190 - DNA, 38 bp, synthetic construct
  source 1..38: mol_type = other DNA
agccaccaccggaaccgcctccctcagagccgccaccc

SEQ ID NO: 191 - DNA, 38 bp, synthetic construct
  source 1..38: mol_type = other DNA
tcagaaccgccaccctcagagccaccaccctcagagcc

SEQ ID NO: 192 - DNA, 38 bp, synthetic construct
  source 1..38: mol_type = other DNA
gccaccagaaccaccaccagagccgccgccagcattga

SEQ ID NO: 193 - DNA, 38 bp, synthetic construct
  source 1..38: mol_type = other DNA
caggaggttgaggcaggtcagacgattggccttgatat

SEQ ID NO: 194 - DNA, 38 bp, synthetic construct
  source 1..38: mol_type = other DNA
tcacaaacaaataaatcctcattaaagccagaatggaa

SEQ ID NO: 195 - DNA, 38 bp, synthetic construct
  source 1..38: mol_type = other DNA
agcgcagtctctgaatttaccgttccagtaagcgtcat

SEQ ID NO: 196 - DNA, 38 bp, synthetic construct
  source 1..38: mol_type = other DNA
acatggcttttgatgatacaggagtgtactggtaataa

SEQ ID NO: 197 - DNA, 38 bp, synthetic construct
  source 1..38: mol_type = other DNA
gttttaacggggtcagtgccttgagtaacagtgcccgt

SEQ ID NO: 198 - DNA, 38 bp, synthetic construct
  source 1..38: mol_type = other DNA
ataaacagttaatgccccctgcctatttcggaacctat

SEQ ID NO: 199 - DNA, 38 bp, synthetic construct
  source 1..38: mol_type = other DNA
tattctgaaacatgaaagtattaagaggctgagactcc

SEQ ID NO: 200 - DNA, 38 bp, synthetic construct
  source 1..38: mol_type = other DNA
tcaagagaaggattaggattagcggggttttgctcagt

SEQ ID NO: 201 - DNA, 38 bp, synthetic construct
  source 1..38: mol_type = other DNA
accaggcggataagtgccgtcgagagggttgatataag

SEQ ID NO: 202 - DNA, 38 bp, synthetic construct
  source 1..38: mol_type = other DNA
tatagcccggaataggtgtatcaccgtactcaggaggt

SEQ ID NO: 203 - DNA, 38 bp, synthetic construct
  source 1..38: mol_type = other DNA
ttagtaccgccaccctcagaaccgccaccctcagaacc

SEQ ID NO: 204 - DNA, 38 bp, synthetic construct
  source 1..38: mol_type = other DNA
gccaccctcagagccaccaccctcattttcagggatag

SEQ ID NO: 205 - DNA, 38 bp, synthetic construct
  source 1..38: mol_type = other DNA
caagcccaataggaacccatgtaccgtaacactgagtt

SEQ ID NO: 206 - DNA, 38 bp, synthetic construct
  source 1..38: mol_type = other DNA
tcgtcaccagtacaaactacaacgcctgtagcattcca

SEQ ID NO: 207 - DNA, 38 bp, synthetic construct
  source 1..38: mol_type = other DNA
cagacagccctcatagttagcgtaacgatctaaagttt

SEQ ID NO: 208 - DNA, 38 bp, synthetic construct
  source 1..38: mol_type = other DNA
tgtcgtctttccagacgttagtaaatgaattttctgta

SEQ ID NO: 209 - DNA, 38 bp, synthetic construct
  source 1..38: mol_type = other DNA
tgggattttgctaaacaactttcaacagtttcagcgga

SEQ ID NO: 210 - DNA, 38 bp, synthetic construct
  source 1..38: mol_type = other DNA
gtgagaatagaaaggaacaactaaaggaattgcgaata

SEQ ID NO: 211 - DNA, 38 bp, synthetic construct
  source 1..38: mol_type = other DNA
ataattttttcacgttgaaaatctccaaaaaaaaggct

SEQ ID NO: 212 - DNA, 38 bp, synthetic construct
  source 1..38: mol_type = other DNA
ccaaaaggagcctttaattgtatcggtttatcagcttg

SEQ ID NO: 213 - DNA, 38 bp, synthetic construct
  source 1..38: mol_type = other DNA
ctttcgaggtgaatttcttaaacagcttgataccgata

SEQ ID NO: 214 - DNA, 38 bp, synthetic construct
  source 1..38: mol_type = other DNA
gttgcgccgacaatgacaacaaccatcgcccacgcata

SEQ ID NO: 215 - DNA, 38 bp, synthetic construct
  source 1..38: mol_type = other DNA
accgatatattcggtcgctgaggcttgcagggagttaa

SEQ ID NO: 216 - DNA, 38 bp, synthetic construct
  source 1..38: mol_type = other DNA
aggccgcttttgcgggatcgtcaccctcagcagcgaaa

SEQ ID NO: 217 - DNA, 38 bp, synthetic construct
  source 1..38: mol_type = other DNA
gacagcatcggaacgagggtagcaacggctacagaggc

SEQ ID NO: 218 - DNA, 38 bp, synthetic construct
  source 1..38: mol_type = other DNA
tttgaggactaaagactttttcatgaggaagtttccat

SEQ ID NO: 219 - DNA, 38 bp, synthetic construct
  source 1..38: mol_type = other DNA
taaacgggtaaaatacgtaatgccactacgaaggcacc

SEQ ID NO: 220 - DNA, 38 bp, synthetic construct
  source 1..38: mol_type = other DNA
aacctaaaacgaaagaggcaaaagaatacactaaaaca

SEQ ID NO: 221 - DNA, 38 bp, synthetic construct
  source 1..38: mol_type = other DNA
ctcatctttgacccccagcgattataccaagcgcgaaa

SEQ ID NO: 222 - DNA, 38 bp, synthetic construct
  source 1..38: mol_type = other DNA
caaagtacaacggagatttgtatcatcgcctgataaat

SEQ ID NO: 223 - DNA, 38 bp, synthetic construct
  source 1..38: mol_type = other DNA
tgtgtcgaaatccgcgacctgctccatgttacttagcc

SEQ ID NO: 224 - DNA, 38 bp, synthetic construct
  source 1..38: mol_type = other DNA
ggaacgaggcgcagacggtcaatcataagggaaccgaa

SEQ ID NO: 225 - DNA, 38 bp, synthetic construct
  source 1..38: mol_type = other DNA
ctgaccaactttgaaagaggacagatgaacggtgtaca

SEQ ID NO: 226 - DNA, 38 bp, synthetic construct
  source 1..38: mol_type = other DNA
gaccaggcgcataggctggctgaccttcatcaagagta

SEQ ID NO: 227 - DNA, 38 bp, synthetic construct
  source 1..38: mol_type = other DNA
atcttgacaagaaccggatattcattacccaaatcaac

SEQ ID NO: 228 - DNA, 38 bp, synthetic construct
  source 1..38: mol_type = other DNA
gtaacaaagctgctcattcagtgaataaggcttgccct

SEQ ID NO: 229 - DNA, 38 bp, synthetic construct
  source 1..38: mol_type = other DNA
gacgagaaacaccagaacgagtagtaaattgggcttga

SEQ ID NO: 230 - DNA, 38 bp, synthetic construct
  source 1..38: mol_type = other DNA
gatggtttaatttcaactttaatcattgtgaattacct

SEQ ID NO: 231 - DNA, 38 bp, synthetic construct
  source 1..38: mol_type = other DNA
tatgcgattttaagaactggctcattataccagtcagg

SEQ ID NO: 232 - DNA, 38 bp, synthetic construct
  source 1..38: mol_type = other DNA
acgttgggaagaaaaatctacgttaataaaacgaacta

SEQ ID NO: 233 - DNA, 38 bp, synthetic construct
  source 1..38: mol_type = other DNA
acggaacaacattattacaggtagaaagattcatcagt

SEQ ID NO: 234 - DNA, 38 bp, synthetic construct
  source 1..38: mol_type = other DNA
tgagatttaggaataccacattcaactaatgcagatac

SEQ ID NO: 235 - DNA, 38 bp, synthetic construct
  source 1..38: mol_type = other DNA
ataacgccaaaaggaattacgaggcatagtaagagcaa

SEQ ID NO: 236 - DNA, 38 bp, synthetic construct
  source 1..38: mol_type = other DNA
cactatcataaccctcgtttaccagacgacgataaaaa

SEQ ID NO: 237 - DNA, 38 bp, synthetic construct
  source 1..38: mol_type = other DNA
ccaaaatagcgagaggcttttgcaaaagaagttttgcc

SEQ ID NO: 238 - DNA, 38 bp, synthetic construct
  source 1..38: mol_type = other DNA
agagggggtaatagtaaaatgtttagactggatagcgt

SEQ ID NO: 239 - DNA, 38 bp, synthetic construct
  source 1..38: mol_type = other DNA
ccaatactgcggaatcgtcataaatattcattgaatcc

SEQ ID NO: 240 - DNA, 38 bp, synthetic construct
  source 1..38: mol_type = other DNA
ccctcaaatgctttaaacagttcagaaaacgagaatga

SEQ ID NO: 241 - DNA, 38 bp, synthetic construct
  source 1..38: mol_type = other DNA
ccataaatcaaaaatcaggtctttaccctgactattat

SEQ ID NO: 242 - DNA, 38 bp, synthetic construct
  source 1..38: mol_type = other DNA
agtcagaagcaaagcggattgcatcaaaaagattaaga

SEQ ID NO: 243 - DNA, 38 bp, synthetic construct
  source 1..38: mol_type = other DNA
ggaagcccgaaagacttcaaatatcgcgttttaattcg

SEQ ID NO: 244 - DNA, 38 bp, synthetic construct
  source 1..38: mol_type = other DNA
agcttcaaagcgaaccagaccggaagcaaactccaaca

SEQ ID NO: 245 - DNA, 38 bp, synthetic construct
  source 1..38: mol_type = other DNA
ggtcaggattagagagtacctttaattgctccttttga

SEQ ID NO: 246 - DNA, 38 bp, synthetic construct
  source 1..38: mol_type = other DNA
taagaggtcatttttgcggatggcttagagcttaattg

SEQ ID NO: 247 - DNA, 38 bp, synthetic construct
  source 1..38: mol_type = other DNA
ctgaatataatgctgtagctcaacatgttttaaatatg

SEQ ID NO: 248 - DNA, 38 bp, synthetic construct
  source 1..38: mol_type = other DNA
caactaaagtacggtgtctggaagtttcattccatata

SEQ ID NO: 249 - DNA, 38 bp, synthetic construct
  source 1..38: mol_type = other DNA
acagttgattcccaattctgcgaacgagtagatttagt

SEQ ID NO: 250 - DNA, 38 bp, synthetic construct
  source 1..38: mol_type = other DNA
ttgaccattagatacatttcgcaaatggtcaataacct

SEQ ID NO: 251 - DNA, 38 bp, synthetic construct
  source 1..38: mol_type = other DNA
gtttagctatattttcatttggggcgcgagctgaaaag

SEQ ID NO: 252 - DNA, 38 bp, synthetic construct
  source 1..38: mol_type = other DNA
gtggcatcaattctactaatagtagtagcattaacatc

SEQ ID NO: 253 - DNA, 38 bp, synthetic construct
  source 1..38: mol_type = other DNA
caataaatcatacaggcaaggcaaagaattagcaaaat

SEQ ID NO: 254 - DNA, 38 bp, synthetic construct
  source 1..38: mol_type = other DNA
taagcaataaagcctcagagcataaagctaaatcggtt

SEQ ID NO: 255 - DNA, 38 bp, synthetic construct
  source 1..38: mol_type = other DNA
gtaccaaaaacattatgaccctgtaatacttttgcggg

SEQ ID NO: 256 - DNA, 38 bp, synthetic construct
  source 1..38: mol_type = other DNA
agaagcctttatttcaacgcaaggataaaaatttttag

SEQ ID NO: 257 - DNA, 38 bp, synthetic construct
  source 1..38: mol_type = other DNA
aaccctcatatattttaaatgcaatgcctgagtaatgt

SEQ ID NO: 258 - DNA, 38 bp, synthetic construct
  source 1..38: mol_type = other DNA
gtaggtaaagattcaaaagggtgagaaaggccggagac

SEQ ID NO: 259 - DNA, 38 bp, synthetic construct
  source 1..38: mol_type = other DNA
agtcaaatcaccatcaatatgatattcaaccgttctag

SEQ ID NO: 260 - DNA, 38 bp, synthetic construct
  source 1..38: mol_type = other DNA
ctgataaattaatgccggagagggtagctatttttgag

SEQ ID NO: 261 - DNA, 38 bp, synthetic construct
  source 1..38: mol_type = other DNA
agatctacaaaggctatcaggtcattgcctgagagtct

SEQ ID NO: 262 - DNA, 38 bp, synthetic construct
  source 1..38: mol_type = other DNA
ggagcaaacaagagaatcgatgaacggtaatcgtaaaa

SEQ ID NO: 263 - DNA, 38 bp, synthetic construct
  source 1..38: mol_type = other DNA
ctagcatgtcaatcatatgtaccccggttgataatcag

SEQ ID NO: 264 - DNA, 38 bp, synthetic construct
  source 1..38: mol_type = other DNA
aaaagccccaaaaacaggaagattgtataagcaaatat

SEQ ID NO: 265 - DNA, 38 bp, synthetic construct
  source 1..38: mol_type = other DNA
ttaaattgtaaacgttaatattttgttaaaattcgcat

SEQ ID NO: 266 - DNA, 38 bp, synthetic construct
  source 1..38: mol_type = other DNA
taaatttttgttaaatcagctcattttttaaccaatag

SEQ ID NO: 267 - DNA, 38 bp, synthetic construct
  source 1..38: mol_type = other DNA
gaacgccatcaaaaataattcgcgtctggccttcctgt

SEQ ID NO: 268 - DNA, 38 bp, synthetic construct
  source 1..38: mol_type = other DNA
agccagctttcatcaacattaaatgtgagcgagtaaca

SEQ ID NO: 269 - DNA, 38 bp, synthetic construct
  source 1..38: mol_type = other DNA
acccgtcggattctccgtgggaacaaacggcggattga

SEQ ID NO: 270 - DNA, 38 bp, synthetic construct
  source 1..38: mol_type = other DNA
ccgtaatgggataggtcacgttggtgtagatgggcgca

SEQ ID NO: 271 - DNA, 38 bp, synthetic construct
  source 1..38: mol_type = other DNA
tcgtaaccgtgcatctgccagtttgaggggacgacgac

SEQ ID NO: 272 - DNA, 38 bp, synthetic construct
  source 1..38: mol_type = other DNA
agtatcggcctcaggaagatcgcactccagccagcttt

SEQ ID NO: 273 - DNA, 38 bp, synthetic construct
  source 1..38: mol_type = other DNA
ccggcaccgcttctggtgccggaaaccaggcaaagcgc

SEQ ID NO: 274 - DNA, 38 bp, synthetic construct
  source 1..38: mol_type = other DNA
cattcgccattcaggctgcgcaactgttgggaagggcg

SEQ ID NO: 275 - DNA, 38 bp, synthetic construct
  source 1..38: mol_type = other DNA
atcggtgcgggcctcttcgctattacgccagctggcga

SEQ ID NO: 276 - DNA, 38 bp, synthetic construct
  source 1..38: mol_type = other DNA
aagggggatgtgctgcaaggcgattaagttgggtaacg

SEQ ID NO: 277 - DNA, 38 bp, synthetic construct
  source 1..38: mol_type = other DNA
ccagggttttcccagtcacgacgttgtaaaacgacggc

SEQ ID NO: 278 - DNA, 46 bp, synthetic construct
  source 1..46: mol_type = other DNA
cagtgccaagcttgcatgcctgcaggtcgactctagaggatctttt

SEQ ID NO: 279 - RNA, 60 nt, SARS-CoV-2
  source 1..60: mol_type = other RNA
atcatatggtttccaacccacttatggtgttggttaccaaccatacagagtagtagtact

SEQ ID NO: 280 - RNA, 60 nt, SARS-CoV-2
  source 1..60: mol_type = other RNA
atcatatggtttccaacccacttctggtgttggttaccaaccatacagagtagtagtact

SEQ ID NO: 281 - RNA, 60 nt, SARS-CoV-2
  source 1..60: mol_type = other RNA
atcatatggtttccaacccacttgtggtgttggttaccaaccatacagagtagtagtact

SEQ ID NO: 282 - DNA, 20 bp, synthetic construct
  source 1..20: mol_type = other DNA
tgggttggaaaccatatgat

SEQ ID NO: 283 - DNA, 20 bp, synthetic construct
  source 1..20: mol_type = other DNA
agtactactactctgtatgg

SEQ ID NO: 284 - DNA, 43 bp, synthetic construct
  source 1..43: mol_type = other DNA
ttcgacaactcgtattaaatcctttgcccgaacgttatttttt

SEQ ID NO: 285 - DNA, 43 bp, synthetic construct
  source 1..43: mol_type = other DNA
gtgagtgaataaccttgcttctgtaaatcgtcgctattttttt

SEQ ID NO: 286 - DNA, 63 bp, synthetic construct
  source 1..63: mol_type = other DNA
tcccaatccaaataagaaacgattttttgtttaacgtctttttttggtaaccaacaccaaaag

SEQ ID NO: 287 - DNA, 14 bp, synthetic construct
  source 1..14: mol_type = other DNA
gtgttggttaccaa

(19) United States
(12) Patent Application Publication
(10) Pub. No.: US 2024/0352522 A1
(43) Pub. Date: Oct. 24, 2024

(54) NUCLEIC ACID DETECTION

(71) Applicant: CAMBRIDGE ENTERPRISE LIMITED, Cambridge, Cambridgeshire (GB)

(72) Inventors: Ulrich KEYSER, Cambridge, Cambridgeshire (GB); Filip BOSKOVIC, Cambridge, Cambridgeshire (GB)

(21) Appl. No.: 18/685,434

(22) PCT Filed: Aug. 23, 2022
(86) PCT No.: PCT/GB2022/052171

§ 371 Date: Feb. 21, 2024

(30) Foreign Application Priority Data
Aug. 23, 2021 (GB) 2112088.6

(51) Int. Cl. C12Q 1/6876; C12Q 1/44; C12Q 1/6825; C12Q 1/70

(52) U.S. Cl. CPC C12Q 1/6876; C12Q 1/44; C12Q 1/6825; C12Q 1/701; G01N 2333/922

(57) ABSTRACT

This invention relates to methods for detecting the presence or absence of target nucleic acids in samples by excising and detecting specific target probes.

The project leading to this application has received funding from the European Research Council (ERC) under the European Union's Horizon 2020 research and innovation programme (grant agreement No 647144).

### FIELD OF THE INVENTION

This invention relates to methods for detecting the presence or absence of target nucleic acid in a sample.

### BACKGROUND OF THE INVENTION

Nucleic acid detection is central to a wide variety of scientific techniques and underpins various detection methods, such as detection of pathogens, genetic conditions and gene expression levels.

Traditional methods for nucleic acid detection typically require amplification of target nucleic acid prior to detection, and so are inherently susceptible to errors induced by enzyme variety, nonspecific amplification of target, amplification biases and limited multiplexing capacity.

The nucleic acid ‘RNA’ is one of four major classes of biomacromolecules in the cell and is a core element of gene expression and gene expression regulation. In addition to its importance for cellular physiology, double and single-stranded RNA forms the genomic material of numerous viruses, including major human pathogenic viruses such as Influenza, Zika, Ebola and SARS-COV-2 which have single-stranded RNA genomes.

Methods traditionally used for detection of RNA include RNA sequencing, Northern blot, and quantitative polymerase chain reaction (qPCR). qPCR is a gold standard method for RNA detection that relies on the real-time fluorescence monitoring of amplified target RNA. In addition to above-mentioned problems associated with target nucleic acid amplification prior to detection, qPCR also requires intensive sample preparation and labour intensive optimization.

As highlighted by the Covid-19 pandemic, there is an urgent need for faster and more reliable nucleic acid detection methods which are less reliant on skilled users and are better-suited to multiplexing, such as for detection of viral RNA and RNA transcripts.

### SUMMARY OF THE INVENTION

The inventors have overcome the above problems by identifying a novel method for detecting the presence or absence of target nucleic acid(s) in a sample. In more detail, the inventors discovered that the presence or absence of target nucleic acids can be determined efficiently and with a high degree of specificity and sensitivity by excising and detecting specific target probe(s) from the target nucleic acid. Advantageously, the method of the invention avoids the need for intensive sample preparation and/or amplification of target nucleic acid(s).

The method of the invention is rapid and can be readily multiplexed allowing the detection of multiple target nucleic acids in a single reaction. The high level of sensitivity and specificity achieved by the invention enables target nucleic acid detection, and optionally quantification, even when present at low concentrations.

The invention provides a method for detecting the presence or absence of a target nucleic acid in a sample, the method comprising the steps of: (a) contacting the sample with a cutting reagent for excising a target probe from the target nucleic acid to provide an excise mixture; (b) contacting the excise mixture with a nucleic acid carrier comprising a capture oligonucleotide that is complementary to the target probe; and (c) detecting binding of the target probe to the capture oligonucleotide; wherein binding of the target probe to the capture oligonucleotide indicates presence of the target nucleic acid in the sample, and the absence of binding of the target probe to the capture oligonucleotide indicates absence of the target nucleic acid in the sample.

In one embodiment, the cutting reagent comprises: (a) cutting oligonucleotides which are complementary to target nucleic acid sequences immediately upstream and immediately downstream of the target probe, and (b) an enzyme suitable for cutting the target nucleic acid at sites hybridised to cutting oligonucleotides.

In one embodiment, the target nucleic acid is RNA, optionally wherein the target nucleic acid is selected from single-stranded RNA, double-stranded RNA, mRNA, miRNA, and non-coding RNA.

In one embodiment, the target nucleic acid is single-stranded RNA and the cutting oligonucleotides comprise single-stranded DNA. In one embodiment, the enzyme suitable for cutting the target nucleic acid at sites hybridised to cutting oligonucleotides is ribonuclease H (RNase H).

In one embodiment, the nucleic acid carrier is a single stranded DNA (ssDNA) carrier. In one embodiment, the nucleic acid carrier comprises one or more reference labels that allow the identity of the nucleic acid carrier, the location of the capture oligonucleotides, and/or the identity of the capture oligonucleotides to be determined.

In one embodiment, the nucleic acid carrier comprises more than one capture oligonucleotide complementary to different target probes, optionally wherein the different target probes are derived from different target nucleic acids.

In one embodiment, the capture oligonucleotide binds to a signalling oligonucleotide in the absence of target probe. In one embodiment, the signalling oligonucleotide is displaced from the capture oligonucleotide in the presence of target probe. In one embodiment, the capture oligonucleotide comprises an overhang that is complementary to the target probe but is not complementary to the signalling oligonucleotide, and wherein in the presence of target probe, the target probe binds to the overhang and displaces the signalling oligonucleotide from the capture oligonucleotide.

In one embodiment, detecting binding of the target probe to the capture oligonucleotide comprises detecting binding of the capture oligonucleotide to the signalling oligonucleotide.

In one embodiment, the signalling oligonucleotide comprises a structural, chemical and/or fluorescent label. In one embodiment, the signalling oligonucleotide comprises a ligand label, and optionally wherein the method further comprises contacting the nucleic acid carrier with a receptor that interacts with the ligand.

In one embodiment, the ligand is biotin and the receptor is avidin, neutravidin, traptavidin or streptavidin, and wherein detecting binding of the capture oligonucleotide to the signalling oligonucleotide comprises detecting the presence of biotin, avidin, neutravidin, traptavidin, streptavidin and/or biotin/avidin, biotin/neutravidin, biotin/traptavidin or biotin/streptavidin complexes.

In one embodiment, the ligand is an antigen and the receptor is an antibody, and wherein detecting binding of the capture oligonucleotide to the signalling oligonucleotide comprises detecting the presence of antigen and/or antigen/antibody complexes.

In one embodiment, the capture oligonucleotide comprises a fluorescent label and the signalling oligonucleotide comprises a quencher and wherein detecting binding of the target probe to the capture oligonucleotide comprises detecting the presence or absence fluorescence.

In one embodiment, binding of the target probe to the capture oligonucleotide is detected using nanopore-based detection methods.

In one embodiment, binding of the target probe to the capture oligonucleotide is detected by spectroscopic-based detection methods.

In one embodiment, the method further comprises quantifying the level of target nucleic acid in the sample by quantifying the level of binding of the target probe to the capture oligonucleotide and/or by quantifying the level of binding of the signalling oligonucleotide to the capture oligonucleotide.

In one embodiment, the method comprises detecting the presence or absence of more than one target nucleic acid in the sample.

In one embodiment, the method comprises excising more than one target probe from the target nucleic acid, optionally from more than one target nucleic acid.

In one embodiment, the target probe has a GC content of 40-60%. In one embodiment, the target probe comprises a terminal region that has a GC content of 40-60%, optionally wherein the terminal region of the target probe is 1 nt, 2 nt, 3 nt, 4 nt, 5 nt, 6 nt, 7 nt, 8 nt, 9 nt, 10 nt, 15 nt, 20 nt, 30 nt, 40 nt, or 50 nt starting from the 3′ and/or the 5′ end of the target probe.

In one embodiment, the target probe has less than 80% sequence identity to other sequences that may be present in the sample, such as other regions of the target nucleic acid.

In one embodiment, the target probe is located in an unhybridized region of the target nucleic acid.

In one embodiment, the target nucleic acid is derived from a virus, optionally wherein the virus is selected from a coronavirus, Influenza virus, Zika virus, Ebola virus, Dengue virus, Hantavirus, Nairovirus, Orthobunyavirus, Phlebovirus, Flavivirus, and Alphavirus. In one embodiment, the target nucleic acid is a coronavirus genome, optionally the SARS-COV-2 genome.

In one embodiment, the target nucleic acid is derived from a microorganism, optionally wherein the target nucleic acid is derived from a bacteria or a fungi.

In one embodiment, the target nucleic acid is derived from a pathogen, optionally wherein the pathogen is a viral pathogen, bacterial pathogen or a fungal pathogen.

In one embodiment, the target nucleic acid is an RNA transcript.

In one embodiment, the target nucleic acid is a therapeutic nucleic acid, optionally wherein the therapeutic nucleic acid is selected from siRNA, shRNA, miRNA, RNA aptamer, DNA aptamer, mRNA, splice-switching oligonucleotides, antisense oligonucleotides, RNA decoys and peptide nucleic acids.

In one embodiment, the target nucleic acid is a genetic biomarker, optionally wherein the target nucleic acid is selected from a gene, an RNA transcript or a region thereof. In one embodiment, the genetic biomarker is associated with a disease or condition, optionally wherein the disease or condition is cancer or an increased risk thereof, or a hereditary disease or condition.

In one embodiment, the target nucleic acid comprises a single nucleotide variant when compared to a reference nucleic acid.

In one embodiment, the sample is obtained from a subject that has been treated with a therapeutic. In one embodiment, the method comprises comparing the level of target nucleic acid in the sample to the level present in a sample from a subject who has not been treated with the therapeutic. In one embodiment, the target nucleic acid is an RNA transcript.

In one embodiment, the sample is obtained from a subject, optionally wherein the subject is a human. In one embodiment, the sample is selected from blood, serum, plasma, saliva, sputum, urine, faeces, cerebrospinal fluid, a lung tissue sample, a bronchoalveolar lavage sample, a nose and/or throat swab sample, or a biopsy sample.

### BRIEF DESCRIPTION OF THE DRAWINGS

FIG. 1. Experimental workflow for SARS-COV-2 RNA detection using toehold-mediated strand displacement. (A) Hybridization of cutting oligonucleotides next to the target probes. (B) RNA in DNA:RNA hybrid is cut with ribonuclease H (RNase H). The result of cutting is five virus-specific probes. (C) Toehold-mediated strand displacement reaction is performed using nucleic acid (e.g. DNA) carrier comprising capture oligonucleotides that are complementary to the target probes. Two references (indicated by black circles) are used to mark the sensing region. A signalling oligonucleotide that is partially complementary to capture oligonucleotides is biotinylated which allows labelling of these sites with streptavidin. Invading probes which are fully complementary to the capture oligonucleotides displace the biotinylated signalling oligonucleotides. (D) Nanopore sensing is employed to discriminate if probes are present. In the absence of the probes, all five sites have the streptavidin-biotin complex that is read as a downward peak in ionic current trace. If the virus probes are present, they will displace the streptavidin-biotin complex and remove the signal in nanopore recordings (the absence of a signal indicates presence of the target probe).

FIG. 2. DNA carrier design was verified with nanopore readout and atomic force microscopy (AFM) imaging. (A) DNA carrier in absence of streptavidin-biotin complex has two distinctive peaks originating from DNA nanostructures or references (indicated by black circles) that can be observed in a sample DNA carrier event. AFM images of DNA carriers are shown, and the single DNA carrier with DNA references is highlighted. (B) DNA carrier event with biotin-streptavidin complexes is depicted and each peak corresponds to the structure present in DNA carrier design. DNA carrier AFM image is shown with each structure marked with a number corresponding to a number in the design.

FIG. 3. SARS-COV-2 strand displacement reaction (SDR) data for ratio dependence on the SDR using RNA and DNA oligonucleotides as shown in (A) and (B) respectively. SDR was performed at room temperature for 10 minutes and each data point is the average of the first fifty unfolded DNA carrier translocations.

FIG. 4. SARS-COV-2 SDR kinetics data for target probes using RNA and DNA oligonucleotides as shown in (A) and (B) respectively.

FIG. 5. SDR results of the detection of SARS-COV-2 probes in human total RNA background. (A) In the negative control measurement, DNA carrier was mixed with human total RNA and incubated in the same SDR conditions as described in Example 1. Each site should have a biotin-streptavidin complex in the absence of the target probe. Hence, for each probe the majority of events should induce a downward peak in nanopore recording as can be observed from example events. (B) The positive control is prepared under the same conditions but for each capture oligonucleotide, 10× excess of target probe is added. As shown, the peaks corresponding to each capture oligonucleotide site do not appear after 5-minute incubation with target probes indicating the absence of the signalling oligonucleotide comprising the biotin-streptavidin complex. The occupied fraction for single sites is plotted in (C) and (D), for negative and positive sample data, respectively. The data clearly show that in the presence of target probes biotin-signalling oligonucleotide is displaced.

FIG. 6. Experimental workflow for MS2 RNA detection using SDR. Here the approach introduced for SARS-COV-2 RNA detection was modified, so that the design has three target sites M1, M2 and M3, instead of five.

FIG. 7. RNase H cutting efficiency was assessed by running samples on 2% (w/v) agarose gel in 1×TBE. On both sides are DNA ladders spanning from around 10 bp to 10 kb. The reactions were prepared to have only viral RNA without (V) or with (E) RNase H cutting protocol without cutting oligonucleotides followed by single probe cutting for M1, M2 and M3, separately and jointly. The mix of all six cutting oligonucleotides was run in the amount used in the RNase H cutting experiment (15 ng) and ten times more (150 ng).

FIG. 8. Denaturing 10% PAGE gel data reveals the length of probes after cutting with cutting oligonucleotide and RNase H and random fragmentation using magnesium ions. MS2 RNA was mixed in 10 mM MgCl₂ and incubated for different periods from κ to 15 minutes. Gel results indicate that even after incubation at 94° C. for 15 minutes, random fragmentation did not achieve desired cutting of short random 20 nt probes. By contrast, samples from the reactions shown in FIG. 7 (demonstrating probe excision by cutting oligonucleotides and RNase H) were also run indicating the efficient excision of the target probes.

FIG. 9. Occupied fraction after SDR with uncut and cut MS2 RNA is shown. (A) Data for the negative control confirm the correct design of DNA carrier as indicated by current peaks which correspond to the presence of biotin-streptavidin complexes at capture oligonucleotide locations on the DNA carrier. (B) After addition of 10 times excess of cut MS2, probes vary in their displacement (absence of signal corresponds to the level of signalling oligo displacement). M1 was shown to be the best site for displacement, which might indicate that it was more successfully cut from the MS2 RNA due to it having the highest percentage likelihood of being unstructured.

FIG. 10. Protocol overview from isolated nucleic acids to nanopore measurement. 4.1 Nanopore sensing with 5 nm pores discriminates single-stranded DNA (probe absent) from double-stranded DNA (probe present) as peaks with a different current drop (peak drop). 4.2 Nanopore sensing with 15 nm pores detects peaks only if a probe is absent i.e. signalling oligonucleotide with structure is not displaced. If signalling oligonucleotide with structure is displaced the probe (i.e. the target nucleic acid) is present (peak absent).

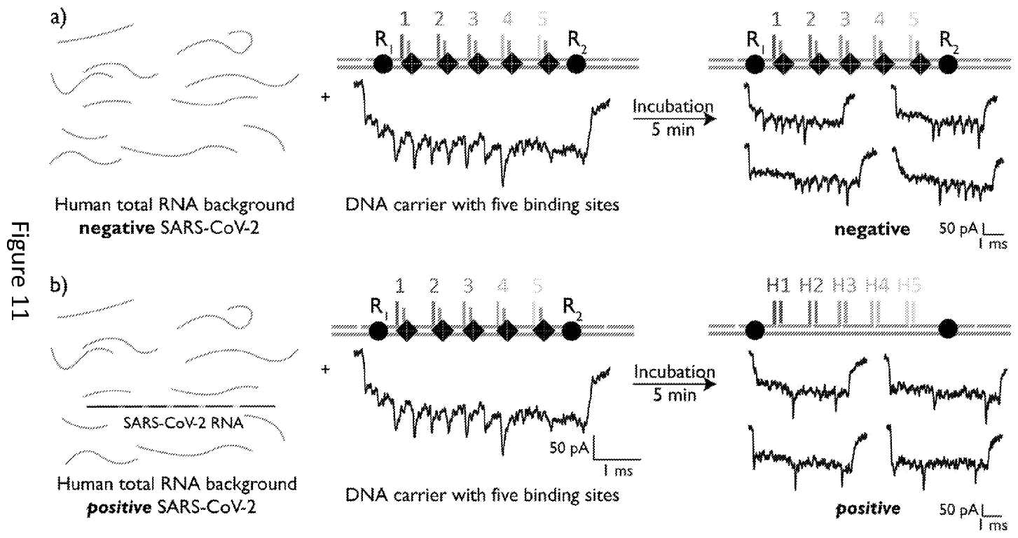

FIG. 11. Results demonstrating the detection of SARS-COV-2 sequences in human total RNA background. (A) Negative control measurements mixing DNA carrier that screens for 5 distinct viral sequences (1-5) in a background of human total RNA, involving screening for five distinct viral sequences that displace five signalling oligonucleotides labelled by streptavidin bound to DNA (black circles). After a 5 minute incubation, the sample containing the DNA carriers is analysed. Four representative events (DNA carrier nanopore current traces) are shown. The DNA carrier signal remains unchanged displaying 7 peaks as before incubation (right) indicating that the sample is negative for the virus. (B) Detection of SARS-COV-2 by strand displacement. Adding SARS-COV-2 probes into the sample at an excess concentration of ^{˜}1 nM (0.3 ng/μL) and incubating for 5 min leads to strand displacement of the signalling oligonucleotides. After incubation, the sample is analysed and the DNA carriers are measured with nanopores. The current trace shows only two peaks signalling the removal of the streptavidin signalling oligonucleotides and indicating the presence of SARS-COV-2 in the sample resulting in positive detection. All measurements were performed in a background of human total RNA at a concentration of 10 ng/μL.

FIG. 12. Human patient swab samples previously tested with RT-PCR and verified with the method of the invention. a) Example nanopore events of DNA carrier using a human patient swab sample detected to be negative for SARS-COV-2 in RT-PCR testing. b) Example nanopore events of DNA carrier using a human patient swab sample detected to be positive for SARS-COV-2 in RT-PCR testing. c) Displacement level for three sites on DNA carrier for negative and positive patient swab samples indicate a clear difference in displacement.

FIG. 13. PAGE gel of RNA cleavage efficiency for probes M1 and M2.

FIG. 14. Quantification of micro RNAs (mR or miRNA) in total RNA extract from Caenorhabditis elegans. Five miRNAs have been analysed: miR-58, miR-1, miR-71, miR-70, miR-72 that correspond to 1, 2, 3, 4, and 5, respectively. A positive sample is distinguishable from a control sample indicating that the method detects miRNAs in a complex transcriptome. Each bar shows the measurement from a separate nanopore while N is the number of events. The presence of these miRNAs was verified previously (Kato, M. et al. Genome Biol 10, R54 (2009)).

FIG. 15. Multiplexed detection of viruses and viral variants with nucleic acid carrier. a) Nucleic acid carrier designed to have five sites specific to SARS-COV-2, influenza A, Respiratory Syncytial Virus (RSV), parainfluenza, and rhinoviruses. Example events in the presence and absence of target probe are depicted. b) Example events in the presence and absence of various SARS-COV-2 variants demonstrating that the method of the invention can discriminate between single-nucleotide variants of SARS-COV-2. c) Displacement levels indicate a clear absence of signal if the corresponding target is present. d) Displacement levels indicate discrimination between single-nucleotide variants (B.1.617, B.1, B.1.1.7, and B.1.351) from wildtype Wuhan strain of SARS-COV-2 virus (reference). Displacement efficiency is calculated for all five sites for multiple nanopores (>3). Error bars represent standard error.

FIG. 16. Discrimination of single-nucleotide SARS-COV-2 RNA variants. a) Representation of two SARS-COV-2 RNA variant controls S:N501T and S:N501S which have a single amino acid substitution due to single nucleotide variant. Cutting oligos are added to the target RNA mixture and anneal next to the target RNA probe. Once annealed, the cutting oligos form RNA:DNA hybrid regions which are cut by RNase H thereby releasing the target RNA probes from the RNA strand. b) DNA carrier design having two references (grey rectangles, annotated ‘1’ and ‘5’) and three capture oligonucleotides for each variant (N501T-annotated ‘2’ and N501S-annotated ‘3’) and the wild-type SARS-COV-2 (annotated ‘4’). The position and substitution of the nucleotide counterparts for each amino acid variant are provided in the table. c) Example nanopore events for no target control indicating correct number of downward spikes each corresponding to a structure depicted in b) (annotated 1-5 as shown in b)). d) and e) Example nanopore events for each N501 variant. The absence of a spike relative to c) indicates the presence of each respective target (e.g. the absence of spike ‘2’ in d) indicates the presence of N501T probes). f) Displacement efficiencies for single-nucleotide SARS-COV-2 variants (labelled as ‘V’) compared with the displacement efficiency for the wild-type SARS-COV-2 (B.1.1.7). Error bars represent standard error for three nanopore measurements and fifty nanopore events per measurement. The difference between conditions with and without variant targets is statistically significant (*** p<0.001; Student's T test; N=150).

### DETAILED DESCRIPTION OF THE INVENTION

The invention provides a method for detecting the presence or absence of target nucleic acid(s) in a sample. The methods of the invention are rapid and exhibit a high degree of sensitivity and specificity. Advantageously, the methods of the invention can be readily multiplexed allowing the simultaneous detection and quantification of multiple target nucleic acids in a single reaction. Moreover, the methods of the invention can advantageously differentiate between closely related sequences in a single sample, e.g. viral or genetic variants such as single-nucleotide variants.

Nucleic acid detection methods known in the art often rely on amplification of the target nucleic acid, or a region thereof. Amplification, e.g. by polymerase chain reaction (PCR), typically requires intensive sample preparation and may exhibit amplification biases, whereby some targets are more efficiently amplified than others. As a result, the specificity and sensitivity of the method varies depending on the target. Amplification biases also reduce the reliability of quantitative nucleic acid detection because targets that are more efficiently amplified appear to be more abundant relative to targets with lower amplification efficiency.

The inventors have overcome these problems by developing a nucleic acid detection method that relies on the detection of specific probes that are excised from the target nucleic acid, without requiring amplification. The target nucleic acid can be detected, and optionally quantified, rapidly and with a high degree of specificity and sensitivity. Advantageously, avoiding amplification of the target nucleic acid avoids the need for intensive sample preparation and reduces the potential for errors introduced by nonspecific amplification and amplification biases.

Existing nucleic acid detection methods may also rely on random fragmentation of nucleic acids to produce shorter fragments which are then detected. During random fragmentation, e.g. by chemical fragmentation or non-specific enzymatic cleavage, all nucleic acids present in a sample (i.e. target and non-target nucleic acids) are non-specifically fragmented, resulting in an abundance of nucleic acid fragments. An abundance of nucleic acid fragments is problematic because, e.g.: (i) it leads to an increased risk of off-target binding between non-target fragments and detection reagents, thereby reducing the specificity of the method and increasing the potential for false-positive results; and (ii) it increases the potential for hybridisation between fragments, thereby preventing or reducing interaction of target nucleic acid(s) with detection reagents, in turn reducing the sensitivity of the method and increasing the potential for false-negative results. Non-specific fragmentation also risks fragmentation of target nucleic acids, thereby reducing the sensitivity and reproducibility of the method, and further increasing the potential for false-negative results.

In overcoming problems with existing methods, the inventors designed a method for excising specific probes (herein “target probe(s)”) from target nucleic acids prior to detection. By limiting the fragmentation of nucleic acids present in a sample, the present invention advantageously reduces the potential for off-target binding with detection reagents and significantly improves the specificity and sensitivity of detection. Excision of specific probes ensures that nucleic acid fragments are generated only in the presence of target nucleic acid. Limiting the generation of non-target probes allows the methods of the invention to retain a high level of sensitivity, even in the presence of total DNA and/or RNA isolated from a patient sample. The methods of the invention also ensure that the target probes have a well-defined sequence allowing detection reagents (nucleic acid carriers and capture oligonucleotides) to be designed with a high level of specificity and reproducibility.

The target probes are designed to maximise the efficiency of: (i) excision of the target probe from the target nucleic acid by the cutting reagents; and (ii) detection of the target probe by capture oligonucleotides. Although existing nucleic acid detection methods often rely on the detection of well-conserved regions of target sequences, the inventors found that probes located in well-conserved regions can exhibit low excision efficiency, thereby reducing the sensitivity of the method. The present invention enables considerable flexibility in designing and optimising selection of target probes and is not restricted to the detection of well-conserved regions of target sequences. Target probes for use in the invention are typically designed for efficient excision from the target nucleic acid and/or for efficient recognition by detection reagents, thereby ensuring a high level of sensitivity and enabling quantification of target nucleic acid(s). A high level of sensitivity ensures that the methods of the invention can reliably detect target nucleic acids at low abundance.

The inventors identified several criteria that may be used to identify target probes that are efficiently excised and detected by the methods of the invention. In some embodiments, target probes have a guanine-cytosine (GC) content of 40-60%. In some embodiments, target probes comprise a terminal region that has a GC content of 40-60%. In some embodiments, target probes have a GC content of 40-60% and a terminal region that has a GC content of 40-60%. A GC content of 40-60% was identified by the inventors as being advantageous because it helps ensure that the probe and the corresponding capture oligonucleotide establish a stable interaction.

In some embodiments, target probes have a high specificity, i.e. low similarity to other nucleic acids that may be present in the sample. Selecting target probes that have high specificity limits the potential for cross-hybridization between the target probe and other nucleic acids. Limiting cross-hybridisation advantageously increases the sensitivity of the methods of the invention because target probes that are excised from the target nucleic acid remain unhybridized and free to interact with detection reagents. Low similarity to other nucleic acids typically means that the target probe has a low level of sequence identity relative to other nucleic acids that may be in the sample, including other regions of the target nucleic acid.

In some embodiments, target probes are located in (or excised from) regions of the target nucleic acid that are unstructured, e.g. regions of the target nucleic acid that are, or are predicted to be, unhybridized. Typically, target probes are located in regions of the target nucleic acid that have the highest likelihood of being unhybridized regions based on published or predicted 3D structures of a target nucleic acid. For single stranded RNA (ssRNA) targets, target probes are typically located in unstructured regions including RNA loops, RNA bulges, and unpaired RNA segments.

### Probe Excision

The invention provides a method for detecting the presence or absence of a target nucleic acid in a sample. The method comprises contacting the sample with a cutting reagent for excising a target probe from the target nucleic acid to provide an excise mixture.

In some embodiments, the cutting reagent comprises: (i) cutting oligonucleotides which are complementary to regions of the target nucleic acid that are directly upstream and downstream of the target probe sequence; and (ii) an enzyme suitable for cutting target nucleic acid hybridised to the cutting oligonucleotides. Cutting oligonucleotides rely on specific base pairing interactions with complementary regions of the target nucleic acid to ‘flank’ the target probe sequence. Enzymatic cutting of the target nucleic acid-cutting oligonucleotide hybrid results in excision of the target probe from the target nucleic acid.

In some embodiments, the target nucleic acid is a ssRNA target and the cutting reagent comprises single stranded DNA (ssDNA) cutting oligonucleotides and ribonuclease H (RNase H). In the presence of ssRNA target, ssDNA cutting oligonucleotides bind to complementary regions of the ssRNA upstream and downstream of the target probe resulting in the formation of RNA:DNA hybrid regions upstream and downstream of the target probe sequence. RNase H cuts RNA in these RNA:DNA hybrid regions by hydrolysing RNA phosphodiester bonds, thereby excising the target probe from the ssRNA target.

Advantageously, the specificity of the cutting reagents allows pre-determined, specific probes to be excised from the target nucleic acid while reducing or avoiding the generation of non-specific nucleic acid fragments which can otherwise interfere with target probes and/or capture oligonucleotides and reduce the sensitivity and/or specificity of the method.

When target nucleic acid is present in the sample, excision of the target probe(s) can occur and so the excise mixture contains excised target probe(s). In the absence of target nucleic acid, the excise mixture does not contain target probe.

The excise mixture may be treated prior to being contacted with the nucleic acid carrier, e.g. to remove or denature enzyme and/or to separate the target probe from longer nucleic acids, such as nucleic acids that are more than 100 nucleotides (nt), more than 200 nt, more than 500 nt, more than 1000 nt, or more than 2000 nt in length. Typically, the target probes are not purified in the excise mixture.

The sample is contacted with cutting reagent under conditions that allow: (i) the cutting oligonucleotides to bind to complementary regions of the target nucleic acid; and (ii) the enzyme to cut the target nucleic acid where the cutting oligonucleotides are bound. The conditions may comprise different phases, e.g. a first phase that allows the cutting oligonucleotides to bind to complementary regions of the target nucleic acid and a second phase that allows the enzyme to cut the target nucleic acid where the cutting oligonucleotides are bound. The cutting oligonucleotide binding phase may comprise incubating the sample with cutting oligonucleotides at a temperature that is optimal for cutting oligonucleotides to anneal to the target nucleic acid. The temperature will vary depending on the nature of the target nucleic acid and cutting oligonucleotides used. The enzymatic cutting phase may comprise incubating at a temperature that is within the optimal activity range for that enzyme, but which does not result in dissociation of the cutting oligonucleotides from the target nucleic acid.

For example, when the enzyme is RNase H, the mixture may be incubated at a temperature in the range of 20-95° C., e.g. at 25-85° C., 25-80° C., 25-75° C., 25-70° C., 25-65° C., 25-60° C., 25-55° C., 25-50° C., 25-45° C., 25-40° C., 30-85° C., 30-80° C., 30-75° C., 30-70° C., 30-65° C., 30-60° C., 30-55° C., 30-50° C., 30-45° C., 30-40° C., 35-40° C. or 36-38° C. In one embodiment, the mixture is incubated at 37° C. Following excision of the target probe, the conditions may further comprise an inactivation phase comprising incubation under conditions that result in the enzyme being inactivated, e.g. to prevent unwanted enzymatic activity during subsequent method steps.

### Target Probes

The terms “probe” and “target probe” are used interchangeably herein to refer to a specific region of the target nucleic acid that is excised from the target nucleic acid and subsequently detected. Reference to “probe” and “probes” should be understood to encompass both a singular probe and multiple (i.e. more than one) probes, unless otherwise indicated.

In some embodiments, the target probe(s) are 10-100 nucleotides (nt) in length, for example 10-90 nt, 10-80 nt, 10-70 nt, 10-60 nt, 10-50 nt, 10-40 nt, 10-35 nt, 10-30 nt, 10-25 nt, 10-20 nt, 15-100 nt, 15-90 nt, 15-80 nt, 15-70 nt, 15-60 nt, 15-50 nt 15-40 nt, 15-35 nt, 15-30 nt, 15-25 nt, 15-20 nt, 20-100 nt, 20-90 nt, 20-80 nt, 20-70 nt, 20-60 nt, 20-50 nt 20-40 nt, 20-35 nt, 20-30 nt, 20-25 nt, 10 nt, 15 nt, 20 nt, 25 nt, 30 nt, 35 nt, 40 nt, 45 nt, 50 nt, 60 nt, 70 nt, 80 nt, 90 nt, or 100 nt in length. In some embodiments, the target probe(s) are 20 nt in length.

The methods of the invention may comprise excising and detecting one probe from a single target nucleic acid. The methods of the invention may comprise excising and detecting one probe from more than one target nucleic acid, e.g. one probe from 2, 3, 4, 5, 6, 7, 8, 9, 10, 11, 12, 13, 14, 15, 20, 25, 30, 40, 50, 60, 70, 80, 90, 100, 150, 200, 250, 500, or 1000 different target nucleic acids. For example, the methods of the invention may comprise excising and detecting one probe from more than one target nucleic acid, e.g. one probe from at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 different target nucleic acids.

The methods of the invention may comprise excising and detecting more than one probe from a single target nucleic acid. For example, the methods of the invention may comprise excising and detecting 2, 3, 4, 5, 6, 7, 8, 9, 10, 11, 12, 13, 14, 15, 20, 25, 30, 40, 50, 60, 70, 80, 90, 100, 150, 200, 250, 500, or 1000 probes from a single target nucleic acid. For example, the methods of the invention may comprise excising and detecting at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 probes from a single target nucleic acid.

The method of the invention may comprise excising and detecting more than one probe from more than one target nucleic acid. For example, the methods of the invention may comprise excising and detecting 2, 3, 4, 5, 6, 7, 8, 9, 10, 11, 12, 13, 14, 15, 20, 25, 30, 40, 50, 60, 70, 80, 90, 100, 150, 200, 250, 500, or 1000 probes from 2, 3, 4, 5, 6, 7, 8, 9, 10, 11, 12, 13, 14, 15, 20, 25, 30, 40, 50, 60, 70, 80, 90, 100, 150, 200, 250, 500, or 1000 different target nucleic acids. For example, the methods of the invention may comprise excising and detecting at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 probes from at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 different target nucleic acids.

To optimise sensitivity, the inventors identified several criteria for designing probes that are susceptible to optimal excision and detection. In some embodiments, target probes: (a) have a guanine-cytosine (GC) content of 40-60%; (b) comprise a terminal region that has a GC content of 40-60%; (c) have a high specificity, i.e. low similarity to other nucleic acids that may be present in the sample to limit cross-hybridization; and/or (d) are located in unstructured (unhybridized) regions of the target nucleic acid.

The invention provides a method of identifying target probes for use in the method of the invention, comprising identifying regions of the target nucleic acid sequence that: (a) have a GC content of 40-60%; (b) comprise a terminal region that has a GC content of 40-60%; (c) have a high specificity, i.e. low similarity to other nucleic acids that may be present in the sample to limit cross-hybridization; and/or (d) are located in unstructured, i.e. unhybridized, regions of the target nucleic acid.

The GC content is the percentage of bases in a nucleic acid molecule that are either guanine (G) or cytosine (C). A probe with a GC content of 40-60% indicates that 40-60% of the bases forming the probe are guanine or cytosine. In some embodiments, probes have a GC content of 40-60%. In some embodiments, probes have a GC content of 40-55%, 40-50% 40-45%, 45-60%, 45-55%, 45-50%, 50-60%, 50-55%, 40%, 45%, 50%, 55% or 60%. In some embodiments, probes have a GC content of less than 40%. In some embodiments, probes have a GC content of more than 60%.

A probe comprising a terminal region that has a GC content of 40-60% means one or both terminal regions of the probe have a GC content of 40-60%. In some embodiments, probes comprise a terminal region with a GC content of 40-55%, 40-50% 40-45%, 45-60%, 45-55%, 45-50%, 50-60%, 50-55%, 40%, 45%, 50%, 55% or 60%. In some embodiments, probes comprise a terminal region with a GC content of less than 40%. In some embodiments, probes comprise a terminal region with a GC content of more than 60%.

In some embodiments, the probe terminal region comprises 1 nt, 2 nt, 3 nt, 4 nt, 5 nt, 6 nt, 7 nt, 8 nt, 9 nt, 10 nt, 15 nt, 20 nt, 30 nt, 40 nt, or 50 nt starting from either the 3′ and/or the 5′ end of the probe. In some embodiments, the probe terminal region comprises 4 nt starting from either the 3′ or the 5′ end of the probe. In some embodiments, the probe terminal region comprises 6 nt starting from the 5′ end of the probe.

In some embodiments, the probe has less than 80%, less than 70%, less than 60%, less than 50%, less than 40%, less than 30%, or less than 20% sequence identity to other nucleic acids that may be present in the sample, such as other regions of the target nucleic acid.

In some embodiments, the target nucleic acid is RNA and the target probe is located in an RNA loop, RNA bulge, or an unpaired RNA segment.

### Cutting Reagent

Target probes are excised from the target nucleic acid by the cutting reagent. In some embodiments, the cutting reagent comprises: (i) cutting oligonucleotides which are complementary to regions of the target nucleic acid that are directly upstream and downstream of the target probe; and (ii) an enzyme suitable for cutting target nucleic acid hybridised to the cutting oligonucleotides. The cutting reagent may comprise multiple cutting oligonucleotides that are complementary to regions upstream and downstream of multiple probes. The cutting reagent may comprise cutting oligonucleotides that are complementary to the full length of the target nucleic acid except the target probe(s).

The terms “cutting oligos” or “cutting oligonucleotides” are used interchangeably herein to refer to short, single stranded nucleic acids which are complementary to regions of the target nucleic acid directly upstream and downstream of the probe. Cutting oligonucleotides base-pair (anneal) with complementary sequences of the target nucleic acid and render these annealed regions susceptible to enzymatic cutting.

In some embodiments, the cutting oligonucleotides are 10-50 nucleotides (nt) in length. In some embodiments, the cutting oligonucleotides are 10-40 nt, 10-35 nt, 10-30 nt, 10-25 nt, 10-20 nt, 15-40 nt, 15-35 nt, 15-30 nt, 15-25 nt, 15-20 nt, 20-40 nt, 20-35 nt, 20-30 nt, 20-25 nt, 10 nt, 15 nt, 20 nt, 25 nt, 30 nt, 35 nt, 40 nt, 45 nt, or 50 nt in length. In some embodiments, the cutting oligonucleotides are 20 nt in length. In some embodiments, the cutting oligonucleotides are more than 50 nt in length, e.g. more than 100 nt, more than 500 nt, more than 1000 nt, more than 1500 nt, or more than 2000 nt in length.

In some embodiments, the cutting oligonucleotides are complementary to the 10-50 nt of the target nucleic acid directly upstream of the target probe. In some embodiments, the cutting oligonucleotides are complementary to the 10-50 nt of the target nucleic acid directly downstream of the target probe. In some embodiments, the cutting reagent comprises cutting oligonucleotides complementary to the 10-50 nt of the target nucleic acid directly upstream of the target probe and cutting oligonucleotides complementary to the 10-50 nt of the target nucleic acid directly downstream of the target probe. In some embodiments, the cutting oligonucleotides are complementary to the 10-40 nt, 10-35 nt, 10-30 nt, 10-25 nt, 10-20 nt, 15-40 nt, 15-35 nt, 15-30 nt, 15-25 nt, 15-20 nt, 20-40 nt, 20-35 nt, 20-30 nt, 20-25 nt, 10 nt, 15 nt, 20 nt, 25 nt, 30 nt, 35 nt, 40 nt, 45 nt, or 50 nt of the target nucleic acid directly upstream and/or directly downstream of the target probe. In some embodiments, the cutting oligonucleotides are complementary to the 20 nt of the target nucleic acid directly upstream and directly downstream of the target probe. In some embodiments, the cutting oligonucleotides are complementary to the full length of the target nucleic acid with the exception of the target probe. The cutting oligonucleotides are not complementary to the target probe.

In some embodiments, the enzyme suitable for cutting target nucleic acid hybridised to the cutting oligonucleotides is a nuclease. In some embodiments, annealing of cutting oligonucleotides to the target nucleic acid forms a double stranded region that is susceptible to cutting by the nuclease. In some embodiments, the target nucleic acid comprises ssRNA, the cutting oligonucleotides comprise ssDNA and the enzyme comprises ribonuclease, e.g. ribonuclease H. When annealed to ssRNA, ssDNA cutting oligonucleotides form RNA:DNA hybrid regions which are recognised and cut by RNase H. RNase H nucleases are non-sequence specific endonucleases which hydrolyse RNA phosphodiester bonds in RNA:DNA hybrids. Cutting of such RNA:DNA hybrid regions by RNase H results in excision of specific target probes from the target nucleic acid.

In some embodiments, cutting oligonucleotides are linked to a nuclease (any suitable linkage may be employed). In such an embodiment, binding of the cutting oligonucleotides to the complementary regions of the target nucleic acid brings the nuclease into close proximity with the target nucleic acid which then excises the probe. In some embodiments, the nuclease is a CRISPR associated (Cas) endonuclease. In some embodiments, the target probe is cut from that target nucleic acid using double specific cleavage using CRISPR-Cas (clustered regularly interspaced short palindromic repeats-CRISPR-associated protein) systems. In this embodiment, the cutting reagent comprises cutting oligos (guide nucleic acids) complexed with Cas nucleases.

In some embodiments, the cutting reagent comprises DNAzymes (deoxyribozymes or DNA enzymes). DNAzymes are single-stranded DNA molecules with catalytic capabilities which can be employed as site-specific RNA cutting enzymes. DNAzymes have two binding arms and a middle catalytic domain that cuts at a specific RNA position. Binding arms can be designed that are specific to regions downstream and upstream of the target probe.

### Identification of Probes

The method comprises contacting the excise mixture with a nucleic acid carrier. The nucleic acid carrier is typically a single stranded nucleic acid to which capture oligonucleotide(s) are bound. In some embodiments, the nucleic acid carrier is a DNA carrier. In some embodiments, the nucleic acid carrier is an RNA carrier. The nucleic acid carrier may be a single stranded DNA (ssDNA) carrier. Nucleic acid carriers may be 100 nucleotides (nt), 200 nt, 500 nt, 1000 nt, 2000 nt, 3000 nt, 4000 nt, 5000 nt, 6000 nt, 7000 nt, 8000 nt, 9000 nt, 10,000 nt or more in length.

The capture oligonucleotide(s) are complementary to the target probe(s). In some embodiments, the capture oligonucleotide(s) comprises a nucleotide that is not complementary to the target probe (i.e. there is a mismatch). In some embodiments, the capture oligonucleotide(s) comprises two, three, four, five, or more mismatches to the target probe.

In some embodiments, the nucleic acid carrier comprises one capture oligonucleotide. In some embodiments, the nucleic acid carrier comprises more than one capture oligonucleotides that are complementary to the same or different target probes. Typically, each capture oligonucleotide is complementary to a single target probe.

In some embodiments, the nucleic acid carrier comprises a single capture oligonucleotide that is complementary to a single target probe. In some embodiments, the nucleic acid carrier comprises more than one capture oligonucleotide complementary to a single target probe e.g. 2, 3, 4, 5, 6, 7, 8, 9, 10, 11, 12, 13, 14, 15, 20, 25, 30, 40, 50, 60, 70, 80, 90, 100, 150, 200, 250, 500, or 1000 capture oligonucleotides which are complementary to a single target probe. In some embodiments, the nucleic acid carrier comprises more than one capture oligonucleotide complementary to a single target probe e.g. at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 capture oligonucleotides which are complementary to a single target probe.

In some embodiments, the nucleic acid carrier comprises capture oligonucleotides that are complementary to single target probes from more than one target nucleic acid, e.g. single target probes from 2, 3, 4, 5, 6, 7, 8, 9, 10, 11, 12, 13, 14, 15, 20, 25, 30, 40, 50, 60, 70, 80, 90, 100, 150, 200, 250, 500 or 1000 target nucleic acids. In some embodiments, the nucleic acid carrier comprises more than one capture oligonucleotide complementary to different probes from a single target nucleic acid, e.g. 2, 3, 4, 5, 6, 7, 8, 9, 10, 11, 12, 13, 14, 15, 20, 25, 30, 40, 50, 60, 70, 80, 90, 100, 150, 200, 250, 500, or 1000 different probes from a single target nucleic acid. The nucleic acid carrier may comprise more than one capture oligonucleotides that are complementary to more than one probe from more than one target nucleic acid, e.g. 2, 3, 4, 5, 6, 7, 8, 9, 10, 11, 12, 13, 14, 15, 20, 25, 30, 40, 50, 60, 70, 80, 90, 100, 150, 200, 250, 500, or 1000 different probes from 2, 3, 4, 5, 6, 7, 8, 9, 10, 11, 12, 13, 14, 15, 20, 25, 30, 40, 50, 60, 70, 80, 90, 100, 150, 200, 250, 500, or 1000 target nucleic acids.

In some embodiments, the nucleic acid carrier comprises capture oligonucleotides that are complementary to single target probes from more than one target nucleic acid, e.g. single target probes from at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500 or at least 1000 target nucleic acids. In some embodiments, the nucleic acid carrier comprises more than one capture oligonucleotide complementary to different probes from a single target nucleic acid, e.g. at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 different probes from a single target nucleic acid. The nucleic acid carrier may comprise more than one capture oligonucleotides that are complementary to more than one probe from more than one target nucleic acid, e.g. at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 different probes from at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 250, at least 500, or at least 1000 target nucleic acids.

The nucleic acid carrier may comprise one or more reference label(s), e.g. structural, chemical or fluorescent label(s). The reference label(s) may be used to identify the nucleic acid carrier and/or to identify the location and/or the identity of capture oligonucleotides.

Capture oligonucleotides typically comprise a region that is complementary to the nucleic acid carrier and a region that is complementary to the target probe. When the region complementary to the nucleic acid carrier is bound thereto, the region complementary to the target probe forms an overhang (i.e. unhybridized) region.

In some embodiments, capture oligonucleotides are 10-100 nucleotides (nt) in length. In some embodiments, capture oligonucleotides are 10-90 nt, 10-80 nt, 10-70 nt, 10-60 nt, 10-50 nt, 10-40 nt, 10-35 nt, 10-30 nt, 10-25 nt, 10-20 nt, 15-100 nt, 15-90 nt, 15-80 nt, 15-70 nt, 15-60 nt, 15-50 nt, 15-40 nt, 15-35 nt, 15-30 nt, 15-25 nt, 15-20 nt, 20-100 nt, 20-90 nt, 20-80 nt, 20-70 nt, 20-60 nt, 20-50 nt, 20-40 nt, 20-35 nt, 20-30 nt, 20-25 nt, 10 nt, 15 nt, 20 nt, 25 nt, 30 nt, 35 nt, 40 nt, 45 nt, 50 nt, 60 nt, 70 nt, 80 nt, 90 nt, or 100 nt in length. Typically, the region of the capture oligonucleotide that is complementary to the target probe is the same length as the corresponding target probe.

In some embodiments, capture oligonucleotides are bound to signalling oligonucleotides that are complementary thereto to form capture-signalling hybrids. In some embodiments, signalling oligonucleotides are 10-100 nt in length. In some embodiments, signalling oligonucleotides are 10-90 nt, 10-80 nt, 10-70 nt, 10-60 nt, 10-50 nt, 10-40 nt, 10-35 nt, 10-30 nt, 10-25 nt, 10-20 nt, 15-100 nt, 15-90 nt, 15-80 nt, 15-70 nt, 15-60 nt, 15-50 nt, 15-40 nt, 15-35 nt, 15-30 nt, 15-25 nt, 15-20 nt, 20-100 nt, 20-90 nt, 20-80 nt, 20-70 nt, 20-60 nt, 20-50 nt, 20-40 nt, 20-35 nt, 20-30 nt, 20-25 nt, 10 nt, 15 nt, 20 nt, 25 nt, 30 nt, 35 nt, 40 nt, 45 nt, 50 nt, 60 nt, 70 nt, 80 nt, 90 nt, or 100 nt in length. In some embodiments, signalling oligonucleotides are 14 nt in length.

The capture oligonucleotide typically has a higher affinity for the target probe than for the signalling oligonucleotide. The affinity between two oligonucleotides may be determined e.g. by the dissociation constant (K_{d}) wherein a lower dissociation constant indicates a higher binding affinity. Two sequences that are fully complementary have a higher binding affinity than two sequences that contain mismatches (i.e. non-complementary nucleotides). Thus, in some embodiments, the capture-signalling hybrid contains at least one mismatch. When there is a mismatch between the target probe and the capture oligonucleotide, particularly the toehold region of the capture oligonucleotide, the strand displacement reaction is slowed because the formation of strand displacement (i.e. the interaction between the probe and the capture-signalling duplex) is destabilised. As a result, probes comprising mismatches can be distinguished from probes that are fully complementary based on the efficiency of strand displacement.

Wherein signalling oligonucleotide is present, the presence of target probe causes displacement of the signalling oligonucleotide from the capture oligonucleotide and/or prevents binding of the signalling oligonucleotide to the capture oligonucleotide. In such embodiments, capture oligonucleotides preferentially interact with target probes via base pairing interactions to form capture-probe hybrids.

Wherein signalling oligonucleotide is present, the absence of target probe (e.g. when the sample does not contain target nucleic acid) typically results in hybridisation of signalling oligonucleotide to the capture oligonucleotide to form capture-signalling hybrids.

Wherein signalling oligonucleotide is not present, the presence of target probe typically results in formation of capture-probe hybrids.

Wherein signalling oligonucleotide is not present, the absence of target probe (e.g. when the sample does not contain target nucleic acid) typically results in unhybridized capture oligonucleotides.

In some embodiments, the capture oligonucleotide in the capture-signalling hybrid comprises an overhang that is complementary to a region (typically a terminal region) of the target probe. An “overhang” refers to at least one unpaired nucleotide. The overhang may be referred to as a toehold region herein. In the presence of target probe, the target probe interacts with the overhang region of the capture oligonucleotide in the capture-signalling hybrid and subsequent base-pairing between complementary bases of the target probe and capture oligonucleotide result in displacement of the signalling oligonucleotide (referred to herein as strand displacement). Displacement of the signalling oligonucleotide results in the formation of capture-probe hybrids. In some embodiments, the capture oligonucleotide comprises a 5′ overhang. In some embodiments, the capture oligonucleotide comprises a 3′ overhang. In some embodiments, the capture oligonucleotide comprises a 5′ overhang and a 3′ overhang. In some embodiments, the overhang is 1 nt, 2 nt, 3 nt, 4 nt, 5 nt, 6 nt, 7 nt, 8 nt, 9 nt, 10 nt, 15 nt, 20 nt, 30 nt, 40 nt, or 50 nt in length. In some embodiments, the overhang is 4 nt in length.

In some embodiments, capture oligonucleotides are contacted with signalling oligonucleotides after being contacted with the excise mixture. In such embodiments, signalling oligonucleotides may bind to capture oligonucleotides that are not already bound to complementary target probes, to form capture-signalling hybrids.

Signalling oligonucleotides comprise labels that can be detected using suitable methods known in the art, e.g. spectroscopic, photochemical, or microscopic methods. In some embodiments, the signalling oligonucleotide comprises a fluorescent label, a chemical label and/or a structural label. In some embodiments, the signalling oligonucleotide comprises a label selected from a nucleic acid nanostructure (e.g. nucleic acid nanostructure, nucleic acid origami structure, hairpin structure or multi-hairpin structure), a ligand (e.g. biotin or an antigen), an enzyme, a radioactive tag and/or a fluorescent tag.

### Detection of Target Probes

The method of the invention comprises detecting binding of the target probe to the capture oligonucleotide; wherein binding of the target probe to the capture oligonucleotide indicates presence of the target nucleic acid in the sample, and the absence of binding of the target probe to the capture oligonucleotide indicates absence of the target nucleic acid in the sample.

The method may comprise detecting binding between the probe and capture oligonucleotide directly, e.g. by detecting the presence or absence of unhybridized (i.e. single stranded) and/or hybridized (i.e. double stranded) capture oligonucleotide(s). In such embodiments, the presence of unhybridized capture oligonucleotide(s) indicates the absence of target probe (and thus absence of the target nucleic acid in the sample), and the presence of hybridized capture oligonucleotides (i.e. capture-probe hybrids) indicates the presence of target probe (and thus presence of the target nucleic acid in the sample).

The method may comprise detecting binding between the probe and capture oligonucleotide indirectly, e.g. by detecting the presence or absence of capture-signalling hybrids. Wherein signalling oligonucleotide is present, absence of capture-signalling hybrids indicates presence of the target probe (and thus presence of the target nucleic acid in the sample). Presence of capture-signalling hybrids indicates absence of the target probe (and thus absence of the target nucleic acid in the sample). The skilled person will understand that the method used to detect the presence or absence of capture-signalling hybrids is dependent on the signalling oligonucleotide label.

In some embodiments, the signalling oligonucleotide is conjugated to a ligand. In this embodiment, presence or absence of capture-signalling hybrids may be determined by detecting the presence or absence of the ligand. In some embodiments, the amount of ligand is quantified and correlated to the amount of target probe and/or the amount of target nucleic acid in the sample.

In some embodiments, the signalling oligonucleotide is conjugated to a ligand and the method further comprises contacting the nucleic acid carrier with a receptor that is specific for said ligand. In this embodiment, presence or absence of capture-signalling hybrids may be determined by detecting the presence or absence of ligand/receptor complexes. In some embodiments, the amount of ligand/receptor complex is quantified and correlated to the amount of target probe and/or the amount of target nucleic acid in the sample.

In some embodiments, the signalling oligonucleotide comprises a biotinylated nucleotide. In some embodiments, the signalling oligonucleotide is covalently attached to biotin. In these embodiments, the method may comprise contacting the nucleic acid carrier with avidin, neutravidin, traptavidin or streptavidin. In this embodiment, the presence or absence of capture-signalling hybrids is determined by detecting the presence or absence of biotin/avidin, biotin/neutravidin, biotin/traptavidin or biotin/streptavidin complexes. Biotin/avidin, biotin/neutravidin, biotin/traptavidin or biotin/streptavidin complexes may be detected using nanopore based methods as described herein.

As used herein, references to avidin encompass neutravidin, traptavidin and streptavidin, and vice versa. Avidin, neutravidin, traptavidin and streptavidin for use in the methods of the invention are typically monomeric or monovalent, although multimeric forms (e.g. divalent trivalent or tetravalent) may also be employed.

In some embodiments, the signalling oligonucleotide is conjugated to an antigen and the method comprises contacting the nucleic acid carrier with an antibody specific for the antigen. In this embodiment, presence or absence of capture-signalling hybrids is determined by detecting the presence or absence of antigen/antibody complexes. In some embodiments, the presence or absence of antigen/antibody complexes is determined by nanopore-based methods.

In some embodiments, the signalling oligonucleotide is conjugated to an enzyme and the method comprises contacting the nucleic acid carrier with a substrate specific for the enzyme. In this embodiment, presence or absence of capture-signalling hybrids is determined by detecting the presence or absence of enzymatic reaction product(s). In some embodiments, the enzyme is horseradish peroxidase (HRP) and substrate is selected from 2,2′-azino-bis(3-ethylbenzothiazoline-6-sulfonic acid) (ABTS), 3,3′,5,5′-Tetramethylbenzidine (TMB), and 3,3′-Diaminobenzidine (DAB). Suitable methods for identifying and quantifying the products of HRP enzyme reactions are known in the art. For example, HRP converts chromogenic substrates, including TMB, DAB and ABTS, into coloured products which can be detected using spectrophotometric methods. The intensity of signal produced by the enzyme substrate may be correlated to the abundance of target probe, and therefore the abundance of target nucleic acid in the sample.

In some embodiments, the capture oligonucleotide is conjugated to a fluorophore and the signalling oligonucleotide is conjugated to a quencher. When the capture oligonucleotide is bound to the signalling oligonucleotide, the fluorophore and quencher are in close proximity and the fluorophore is quenched, i.e. does not fluoresce. In the presence of target probe, the signalling oligonucleotide is displaced and the fluorophore and quencher are separated resulting in the fluorophore fluorescing. In some embodiments, the fluorophore is a fluorescein, such as 6-carboxyfluorescein (6-FAM), and the quencher is Dabcyl or tetramethylrhodamine. The presence or absence of fluorescence can be detected by methods known in the art, e.g. fluorescence spectroscopy. The presence of fluorescence indicates that the signalling oligonucleotide has been displaced or prevented from binding to the capture oligonucleotide, thereby indicating the presence of capture-probe hybrids. The intensity of fluorescence may be quantified and correlated to the abundance of probe, and therefore to the abundance of target nucleic acid in the sample.

The presence or absence of capture-signalling hybrids may be detected using nanopore-based methods. In nanopore-based detection methods, an ionic current passes through the nanopore due to an applied potential. When nucleic acid carriers translocate through a nanopore, a current signature or current trace is produced which corresponds to the current level detected over time as the nucleic acid carrier translocates through the nanopore. The current signature may be compared to a negative control (e.g. a current trace produced by the nucleic acid carrier in the absence of target probe and/or presence of signalling oligonucleotide); and/or to a positive control (e.g. a current trace produced by the nucleic acid carrier in the presence of target probe and/or absence of signalling oligonucleotide). A current signature or current trace may also be referred to herein as an event or a nanopore event.

The nucleic acid carrier may comprise one or more reference labels or identification labels that produce an identifiable signal in the current trace. Reference labels, and the current signals they produce, may be used to locate and/or identify capture oligonucleotides. This is advantageous because it allows capture-signalling hybrids to be differentiated, even when the same signalling labels are used. Identification labels, and the current signals they produce, may be used to identify the nucleic acid carrier. Advantageously, identification labels allow nucleic acid carriers from multiple reactions to be combined in a single nanopore-based detection assay.

Wherein a nanopore is used for detecting binding of the target probe to the capture oligonucleotide, the type of nanopore used will depend on whether the binding is being detected directly (e.g. by identifying single stranded capture oligonucleotides and double stranded capture-probe hybrids) or indirectly (e.g. by detecting capture-signalling hybrids). The nanopore may be a solid state or a biological nanopore. In some embodiments, the nanopore is a glass nanopore. For direct detection of capture-probe binding, nanopores with a diameter of about 3 nm, 4 nm, 5 nm, 6 nm, 7 nm, 8 nm, 9 nm, or 10 nm are typically used. For example, direct detection of capture-probe binding, nanopores with a diameter of about 3 nm-about 10 nm, about 3 nm-about 9 nm, about 3 nm-about 8 nm, about 3 nm-about 7 nm, about 3 nm-about 6 nm, about 3 nm-about 5 nm, about 3 nm-about 4 nm, about 4 nm-about 10 nm, about 4 nm-about 9 nm, about 4 nm-about 8 nm, about 4 nm-about 7 nm, about 4 nm-about 6 nm, about 4 nm-about 5 nm, about 5 nm-about 10 nm, about 5 nm-about 9 nm, about 5 nm-about 8 nm, about 5 nm-about 7 nm, about 5 nm-about 6 nm, about 6 nm-about 10 nm, about 6 nm-about 9 nm, about 6 nm-about 8 nm, about 6 nm-about 7 nm, about 7 nm-about 10 nm, about 7 nm-about 9 nm, about 7 nm-about 8 nm, about 8 nm-about 10 nm, about 8 nm-about 9 nm, or about 9 nm-about 10 nm, are typically used. For indirect detection of capture-probe binding, nanopores with a diameter of about 10 nm, 11 nm, 12 nm, 13 nm, 14 nm, 15 nm, 16 nm, 17 nm, 18 nm, 19 nm, or 20 nm are typically used. For example, for indirect detection of capture-probe binding, nanopores with a diameter of about 10 nm-about 20 nm, about 10 nm-about 19 nm, about 10 nm-about 18 nm, about 10 nm-about 17 nm, about 10 nm-about 16 nm, about 10 nm-about 15 nm, about 10 nm-about 14 nm, about 10 nm-about 13 nm, about 10 nm-about 12 nm, about 10 nm-about 11 nm, about 11 nm-about 20 nm, about 11 nm-about 19 nm, about 11 nm-about 18 nm, about 11 nm-about 17 nm, about 11 nm-about 16 nm, about 11 nm-about 15 nm, about 11 nm-about 14 nm, about 11 nm-about 13 nm, about 11 nm-about 12 nm, about 12 nm-about 20 nm, about 12 nm-about 19 nm, about 12 nm-about 18 nm, about 12 nm-about 17 nm, about 12 nm-about 16 nm, about 12 nm-about 15 nm, about 12 nm-about 14 nm, about 12 nm-about 13 nm, about 13 nm-about 20 nm, about 13 nm-about 19 nm, about 13 nm-about 18 nm, about 13 nm-about 17 nm, about 13 nm-about 16 nm, about 13 nm-about 15 nm, about 13 nm-about 14 nm, about 14 nm-about 20 nm, about 14 nm-about 19 nm, about 14 nm-about 18 nm, about 14 nm-about 17 nm, about 14 nm-about 16 nm, about 14 nm-about 15 nm, about 15 nm-about 20 nm, about 15 nm-about 19 nm, about 15 nm-about 18 nm, about 15 nm-about 17 nm, about 15 nm-about 16 nm, about 16 nm-about 20 nm, about 16 nm-about 19 nm, about 16 nm-about 18 nm, about 16 nm-about 17 nm, about 17 nm-about 20 nm, about 17 nm-about 19 nm, about 17 nm-about 18 nm, about 18 nm-about 20 nm, about 18 nm-about 19 nm, or about 19 nm-about 20 nm are typically used. The skilled person will readily understand that the diameter of the nanopore used will depend on the size of the label that is associated with the signalling oligonucleotide.

A biological nanopore may be a transmembrane protein nanopore. Examples of transmembrane protein pores include β-barrel pores and α-helix bundle pores. β-barrel pores comprise a barrel or channel that is formed from β-strands. β-barrel pores include, but are not limited to, β-toxins, such as α-hemolysin (α-HL), anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. α-helix bundle pores include, but are not limited to, inner membrane proteins and a outer membrane proteins, such as WZA and ClyA toxin. A biological nanopore may be a transmembrane pore derived from or based on MspA, α-HL, lysenin, CsgG, ClyA, or haemolytic protein fragaceatoxin C (FraC).

Examples of transmembrane pores derived from or based on MspA are described in WO 2012/107778. Examples of transmembrane pores derived from or based on α-hemolysin are described in WO 2010/109197. Examples of transmembrane pores derived from or based on lysenin are described in WO 2013/153359. Examples of transmembrane pores derived from or based on CsgG are described in WO 2016/034591 and WO 2019/002893. Examples of transmembrane pores derived from or based on ClyA are described in WO 2017/098322. Examples of transmembrane pores derived from or based on FraC are described in WO 2020/055246.

The nanopore may be a DNA origami pore. Examples of DNA origami pores are described in WO 2013/083983, WO 2018/011603, and WO 2020/025974.

The nanopore may be a solid state nanopore. Examples of solid state nanopores are described in WO 2016/127007.

In nanopore-based detection methods, signalling oligonucleotides typically comprise a structural label that produces an identifiable current signal, i.e. reduction in current, when translocated through the nanopore. The number of capture-signalling hybrids that are detected may be quantified and correlated to the amount of target probe and/or target nucleic acid in the sample.

In some embodiments, signalling oligonucleotides comprise a biotin label and capture-signalling hybrids are detected by detecting the presence or absence of biotin using nanopore-based detection methods. In some embodiments, the capture oligonucleotides are further contacted with streptavidin, neutravidin, traptavidin or avidin and capture-signalling hybrids are detected by detecting the presence or absence of biotin/streptavidin, biotin/neutravidin, biotin/traptavidin or biotin/avidin complexes using nanopore-based detection methods.

In some embodiments, signalling oligonucleotides comprise an avidin, neutravidin, traptavidin or streptavidin label and capture-signalling hybrids are detected by detecting the presence or absence of avidin, neutravidin, traptavidin or streptavidin using nanopore-based detection methods. In some embodiments, the capture oligonucleotides are further contacted with biotin and capture-signalling hybrids are detected by detecting the presence or absence of biotin/streptavidin, biotin/neutravidin, biotin/traptavidin or biotin/avidin complexes using nanopore-based detection methods.

The size and duration of current blockages (as represented by reduction(s) in current) can be used to differentiate between different signalling labels because the size of the current blockage is typically relative to the size of the label (larger labels typically produce larger peaks/greater reductions in current, and vice versa). Multiple labels corresponding to multiple different capture-signalling hybrids can therefore be identified in a single reaction.

### Quantification of Target Nucleic Acids

The superior sensitivity of the method of the invention advantageously allows for quantitative detection of target nucleic acids. As described in detail above, the methods of the invention comprise detecting the presence or absence of target probes which (if present) are derived directly from the target nucleic acid. Advantageously, the abundance of target probes is not altered, (e.g. by prior amplification) in the method of the invention and so the abundance of target probe corresponds directly to the abundance of target nucleic acid.

The methods of the invention may comprise calibrating the level of capture-probe or capture-signalling hybrids with the abundance of target probe present in the sample. The sample may be contacted with a known amount of capture oligonucleotides and the difference between the level of capture-signalling hybrids present in the sample and the level present in a negative control (e.g. in the absence of target probe) may be used to determine the abundance of target probe, and therefore the abundance of target nucleic acid in the sample. For example, a 50% reduction in the level of capture-signalling hybrids relative to the negative control indicates that 50% of capture oligonucleotides have interacted with probe to form capture-probe hybrids. The number of capture-probe hybrids detected corresponds to the abundance of target nucleic acid present in the sample.

Advantageously, the method of the invention can be readily adapted to allow detection of target nucleic acids in a wide variety of concentration ranges. Probes can be designed to optimise the dynamic range (the range of target nucleic acid concentration that can be detected) by altering: (i) the toehold length wherein a shorter toehold length typically increases the dynamic range; (ii) the GC content wherein a lower GC content increases the dynamic range; (iii) the length of the probe wherein shorter probes increase the dynamic range; and/or (iv) alternating the position of the toehold wherein a 5′ toehold exhibits faster displacement than a 3′ toehold and therefore decreases the dynamic range. Decreasing the efficiency of strand displacement typically increases the dynamic range of the assay because a more concentrated sample is required to achieve equivalent strand displacement rates.

The methods of the invention can be readily multiplexed by employing signalling oligonucleotides with distinguishable labels or by differentiating between different capture oligonucleotides using nucleic acid carrier reference labels. In these embodiments, the relative and/or absolute abundance of multiple target nucleic acid(s) can be measured in a single reaction.

### Target Nucleic Acids

As used herein, the term “target nucleic acid” encompasses a single target nucleic acid and multiple (i.e. more than one) target nucleic acids. The target nucleic acid may comprise RNA, e.g. single-stranded RNA (ssRNA) or double-stranded RNA (dsRNA), or DNA, e.g. single-stranded DNA (ssDNA) and double-stranded DNA (dsDNA), or combinations thereof. The target nucleic acid may be messenger RNA (mRNA), microRNA (miRNA), non-coding RNA, small interfering RNA (siRNA), short hairpin RNA (shRNA) or ribosomal RNA (rRNA). The target nucleic acid may be autosomal DNA, or mitochondrial DNA. The target nucleic acid may be a naturally occurring or synthetic nucleic acid.

When detecting the presence or absence of double-stranded target nucleic acid, the method may further comprise denaturing the target nucleic acid to produce single-stranded nucleic acid prior to contacting the target nucleic acid with cutting reagents.

The method of the invention may be used to detect the presence or absence of more than one target nucleic acid in a sample. For example, the method of the invention may be used to detect the presence or absence of 2, 3, 4, 5, 6, 7, 8, 9, 10, 11, 12, 13, 14, 15, 20, 25, 30, 40, 50, 60, 70, 80, 90, 100, 150, 200, 250, 500, or 1000 target nucleic acids in a sample.

In some embodiments, the sample contains the target nucleic acid. In some embodiments, the sample does not contain target nucleic acid. In some embodiments, the sample comprises non-target nucleic acid(s). The sample may be obtained from a cell culture. The sample may be obtained from a subject.

The subject may be selected from a human or a non-human animal, such as a murine, bovine, equine, ovine, canine, or feline animal. The sample may be selected from the group consisting of, but not limited to, blood, serum, plasma, saliva, sputum, urine, faeces, cerebrospinal fluid, a lung tissue sample, a bronchoalveolar lavage sample, a nose and/or throat swab sample, or a biopsy sample.

The sample may be treated prior to use in the method of the invention. For example, the sample may be treated to lyse cells, remove and/or denature proteins. Nucleic acid extraction may be performed on the sample prior to use in the method of the invention. Suitable nucleic acid extraction methods are known in the art and include methods that extract total DNA and/or RNA from samples.

### Pathogen Detection

Methods of the invention may be used to detect the presence or absence of target nucleic acids derived from a pathogen. Presence of a target nucleic acid derived from a pathogen in the sample typically indicates presence of the pathogen in the sample, whereas absence of the target nucleic acid in the sample typically indicates absence of the pathogen in the sample. Methods of the invention may be used to detect target nucleic acid derived from a viral pathogen, a bacterial pathogen, or a fungal pathogen.

The target nucleic acid may be viral nucleic acid, e.g. a viral genome, such as a ssRNA viral genome. The ssRNA viral genome may be derived from a virus selected from an Influenza virus, Zika virus, Ebola virus, coronavirus, Dengue virus, Hantavirus, Nairovirus, Orthobunyavirus, Phlebovirus, Flavivirus, and Alphavirus. In some embodiments, the target nucleic acid is derived from a coronavirus, such as SARS-CoV-2.

The nucleic acid carrier may comprise multiple capture oligonucleotides that are specific for different probes derived from the same pathogen. Alternatively, the nucleic acid carrier may comprise multiple capture oligonucleotides that are specific for probe(s) derived from different pathogens.

In some embodiments, target probes are designed to detect the presence or absence of a particular pathogen, and/or to detect the presence or absence of particular pathogen variants. For example, probes that are common to a group of pathogens (e.g. SARS-COV-2) may be used to detect the presence or absence of a pathogen within that group; and probes that are specific to a particular variant may be used to detect the presence or absence of different variants.

In some embodiments, methods of the invention are used to quantify the relative abundance of multiple pathogens in the sample. Advantageously, the methods of the invention may be used to identify the predominant pathogen, or pathogen variant, in the sample.

### Genetic Marker Detection

Methods of the invention may be used to detect the presence or absence of a genetic biomarker, e.g. a genetic variant. The genetic biomarker may be associated with a particular disease or condition, or an increased risk thereof. For example, the genetic biomarker may be associated with cancer, or an increased risk thereof. The genetic biomarker may be associated with a hereditary disease or condition. The presence of the genetic biomarker may indicate the presence of a disease or condition, or increased risk of a disease or condition, that is associated with that biomarker. The absence of the genetic biomarker may indicate the absence of a disease or condition, or reduced risk of a disease or condition, that is associated with that biomarker.

In methods for detecting the presence or absence of a genetic biomarker, the target nucleic acid may be the genome of a subject, or a region thereof, a gene or a region thereof, or may be an RNA transcript produced by a gene of interest. The method may comprise detecting the presence or absence, and optionally the relative abundance, of multiple variants of a particular genetic marker in a single reaction.

Nucleic acid carriers may comprise capture oligonucleotides that are specific for all known variants of a genetic marker.

### SNV Detection

Detection of target nucleic acid(s) relies on specific base-pairing between target probes and capture oligonucleotides which are complementary thereto. Target probes and capture oligonucleotides that are fully complementary establish a more stable interaction than capture-probe pairs containing mismatches. Methods of the invention can therefore be used to differentiate between highly similar sequences, e.g. target nucleic acids containing single nucleotide variants (SNVs). This is particularly advantageous because the method of the invention can be used as a screening method for the detection of SNVs, and SNV-based biomarkers. The region of the capture oligonucleotide(s) that is specific to a particular SNV may be positioned in the toehold overhang region of capture-signalling hybrids.

In some embodiments, the target nucleic acid comprises a SNV when compared to a reference nucleic acid. The reference nucleic acid may be the wild type form of the target nucleic acid. In some embodiments, the target nucleic acid is a gene or a region thereof comprising a SNV when compared to the wild type form of the gene.

### Transcriptomics

Methods of the invention may be used to detect the presence or absence, and optionally the abundance, of RNA transcript(s). Thus, methods of the invention may be used in transcriptomics, i.e. to detect the presence or absence, and optionally the abundance, of RNA transcripts in a sample. The method of the invention may be used to measure the presence or absence, and optionally the abundance, of transcripts derived from a single cell (e.g. single cell transcriptomics).

### Pharmacokinetics and Drug Screening

Methods of the invention may be used to detect the presence or absence, and optionally the abundance, of therapeutic nucleic acids in a sample. Therapeutic nucleic acids are typically administered to a subject to treat a disease or condition. For example, the therapeutic nucleic acid may be selected from siRNA, shRNA, miRNA, RNA or DNA aptamers, mRNA, splice-switching oligonucleotides, antisense oligonucleotides, RNA decoys and peptide nucleic acids. Methods of the invention may be used to detect the presence or absence of therapeutic nucleic acids in samples obtained at different time points and/or from different tissues or locations allowing the pharmacokinetics of the therapeutic nucleic acid to be tracked.

Methods of the invention may also be used to detect the presence or absence, and optionally the abundance, of native nucleic acids in the presence or absence of treatment with a drug candidate designed to target the native nucleic acid. For example, a patient may be administered a drug (e.g. a therapeutic nucleic acid) that is designed to lower the expression of a particular gene or to reduce the abundance of the corresponding gene transcript. The methods of the invention may be used to detect the presence or absence, and optionally the abundance, of the particular gene transcript in samples obtained in the presence or absence of the drug candidate to determine the activity of the drug on the gene transcript. Samples may be obtained from the subject at different time points and/or from different tissues or locations to determine the pharmacokinetics of the drug candidate.

Methods of the invention may be used to screen drug candidates for their ability to reduce or increase the abundance of gene transcripts in a sample. For example, various cell cultures may be treated with putative drug candidates. Following treatment, the methods of the invention may be used to detect the abundance of the gene transcript of interest in the various cell cultures.

### EXAMPLES

The invention will be further clarified by the following examples, which are intended to be purely exemplary of the invention and are in no way limiting.

### Example 1

The inventors developed a method for detecting the presence of SARS-COV-2 and Escherichia virus MS2 target nucleic acids. As shown herein, the inventors designed DNA carriers for detecting the presence or absence of short RNA probes that are recognised and excised from SARS-COV-2 and MS2. The DNA carrier was assembled by annealing capture oligonucleotides that are complementary to regions of SARS-COV-2 and MS2 to single-stranded DNA. The inventors found that multiple RNA probes derived from SARS-COV-2 and MS2 could be detected in-parallel, even at ultralow concentrations. In the present example, ultrasensitive nanopore based detection methods were used to identify the presence or absence of capture-signalling hybrids. The design of DNA carrier structures was verified with AFM imaging and gel electrophoresis.

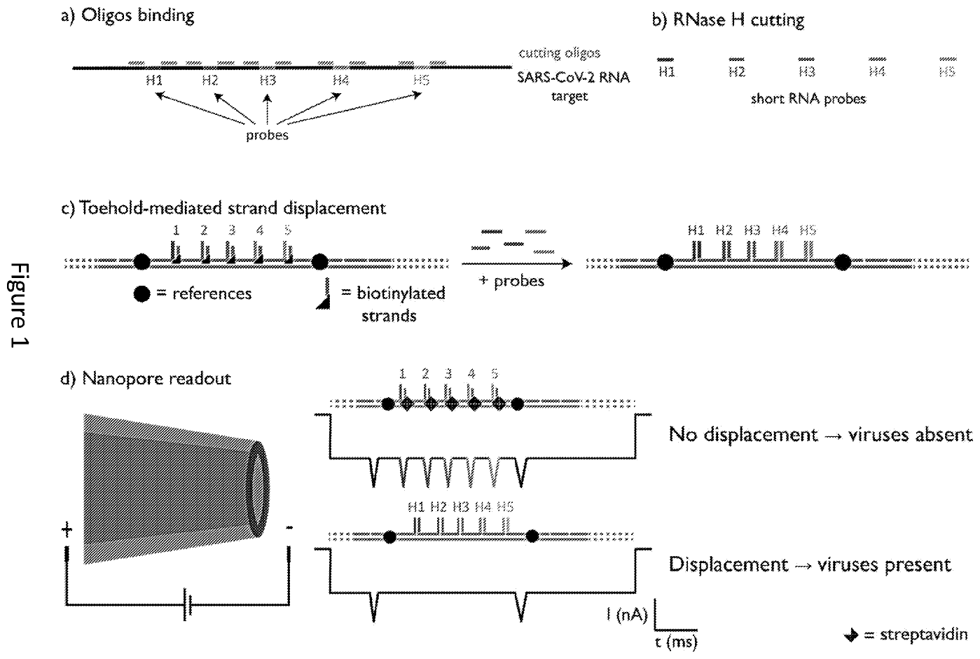

An overview of the experimental design for viral RNA detection is shown in FIG. 1. The process includes four steps:
(1) cutting oligonucleotides complementary to viral RNA regions upstream and downstream of the target probes are bound to the RNA virus (in FIG. 1A, the SARS-COV-2 genome is shown);
(2) RNA:DNA hybrids formed by cutting oligonucleotides binding to the viral RNA are cut with RNase H resulting in the release of target probes (FIG. 1B);
(3) the presence/absence of the target probes is detected using a DNA carrier with capture-signalling hybrids annealed thereto and SDRs. The DNA carrier has five overhangs each specific to a target probe (H1-H5 in FIG. 1) and each comprising a bound biotinylated signalling oligonucleotide which is partially complementary to the overhang, but has a 6 nt toehold end that serves as a seed for strand displacement by fully complementary target probes. If probes are present in the mixture the biotinylated signalling oligonucleotide will be displaced (FIG. 1C)
(4) nanopore sensing is used to discriminate the two possible states (i.e. the presence or absence of the target probe, FIG. 1D) after adding monovalent streptavidin the non-displaced biotinylated signalling oligonucleotides induce a downward peak specific to their position in the DNA carrier. The DNA carrier also comprises references composed of eight DNA nanostructures that are present in both cases and mark the sensing region of DNA carrier.

### Identification of Probes and Corresponding Cutting Oligonucleotides

The SARS-COV-2 probes and MS2 probes used in the present example are provided in Tables 1 and 2, respectively.

TABLE 1
Sequence of 20 nt RNA and DNA probes
from the SARS-COV-2 RNA genome.

| SEQ | | Nucleic | | |
|---|---|---|---|---|
| ID | | acid | Target RNA/DNA | Length |
| NO | Code | type | probe (5′-3′) | (nt) |
| 1 | H1 | RNA | UGAUUGUGAAGAAGAAGAGU | 20 |
| 2 | | DNA | TGATTGTGAAGAAGAAGAGT | |
| 3 | H2 | RNA | AAGAAAGGAGCUAAAUUGUU | 20 |
| 4 | | DNA | AAGAAAGGAGCTAAATTGTT | |
| 5 | H3 | RNA | AGAGUUGAUUUUUGUGGAAA | 20 |
| 6 | | DNA | AGAGTTGATTTTTGTGGAAA | |
| 7 | H4 | RNA | UGGUGUUUAUUCUGUUAUUU | 20 |
| 8 | | DNA | TGGTGTTTATTCTGTTATTT | |
| 9 | H5 | RNA | GGUAAAGUUGAGGGUUGUAU | 20 |
| 10 | | DNA | GGTAAAGTTGAGGGTTGTAT | |

TABLE 2
Target probe sequences, with length of
20 nt, from the MS2 RNA genome.

| SEQ | | | |
|---|---|---|---|
| ID | | Target RNA/DNA | Length |
| NO | Code | probe (5′-3′) | (nt) |
| 11 | M1 | ACCACTAATGAGTGATATCC | 20 |
| 12 | M2 | TACCTGTAGGTAACATGCTC | 20 |
| 13 | M3 | TCTGCATCCGATTCCATCTC | 20 |
| 14 | M4 | CCTGATATGAATATGTACCT | 20 |
| 15 | M5 | ACCTCCCCCTAAAGAGAGGA | 20 |

To excise the probes from SARS-COV-2 and MS2 RNA, two complementary cutting oligonucleotides are hybridised upstream and downstream of the probe of interest. Cutting oligonucleotides for the MS2 probes in Table 2 are provided in Table 3, respectively.

TABLE 3
MS2 probe cutting DNA
oligonucleotides.

| SEQ | | | |
|---|---|---|---|
| ID | | Cutting oligo | Length |
| NO | Code | (5′-3′) | (nt) |
| 16 | M1a | AACCAACCGAACTGCAACTC | 20 |
| 17 | M1b | AAGCATCTCATATGCACCCT | 20 |
| 18 | M2a | GGAGCCAGTCGACAACGAAT | 20 |
| 19 | M2b | ACGGGGGCCGTAAGGCCCTC | 20 |
| 20 | M3a | CGATAAGTCTATCGTCGCAA | 20 |
| 21 | M3b | AACTCCACACCAGGCGATCG | 20 |

### DNA Carrier Assembly

To make the DNA carrier, a long linear single-stranded DNA scaffold was annealed with short complementary oligonucleotides (purchased from Integrated DNA Technologies) (Table 22). To prepare the linear scaffold, single-stranded circular M13mp18 DNA (7249 nt, Guild Biosciences, USA) was cleaved by restriction enzymes after binding to a short oligonucleotide (39 nt) which created double-stranded restriction sites (see the protocol details provided in Bell, N. A. W. & Keyser, U. F. Nat. Nanotechnol. 11, 645-651 (2016)). The oligonucleotide set for making a specific design was prepared by mixing the required oligonucleotides (with 200 nM final concentration of each in the mixture).

To produce DNA carriers for the detection of the SARS-COV-2 and MS2, several fully complementary oligonucleotides (i.e. those listed in Table 22) were replaced with capture oligonucleotides which comprise a region complementary to the DNA scaffold and an overhang region complementary to the target probe. Capture oligonucleotides for the SARS-COV-2 and MS2 virus probes and the fully complementary oligonucleotides which they replace are listed in Tables 4 and 5, respectively. The oligonucleotides used to produce reference signals and the corresponding complementary oligonucleotides they replaced are listed in Table 6.

TABLE 4
Capture DNA oligonucleotides corresponding to
SARS-COV-2 probes and the DNA carrier comple-
mentary oligonucleotides that are replaced
therewith to form the SARS-COV-2 DNA carrier.
The region of the capture oligo complementary
to the corresponding target probe is in bold.

| SEQ | | | Oligonu- |
|---|---|---|---|
| ID | | | cleotide |
| NO | Code | Capture oligo (5′-3′) | replaced |
| 22 | cH1_42 | TTCGACAACTCGTATTAAAT | SEQ ID |
| | | CCTTTGCCCGAACGTTAT | NO: 42 |
| | | TTTTT | |
| | | ACTCTTCTTCTTCACAATCA | |
| 23 | cH2_55 | GTGAGTGAATAACCTTGCTT | SEQ ID |
| | | CTGTAAATCGTCGCTATT | NO: 55 |
| | | TTTTT | |
| | | AACAATTTAGCTCCTTTCTT | |
| 24 | cH3_68 | AGAATATAAAGTACCGACAA | SEQ ID |
| | | AAGGTAAAGTAATTCTGT | NO: 68 |
| | | TTTTT | |
| | | TTTCCACAAAAATCAACTCT | |
| 25 | cH4_81 | TCCCAATCCAAATAAGAAAC | SEQ ID |
| | | GATTTTTTGTTTAACGTC | NO: 81 |
| | | TTTTT | |
| | | AAATAACAGAATAAACACCA | |
| 26 | cH5_94 | CATTCAACCGATTGAGGGAG | SEQ ID |
| | | GGAAGGTAAATATTGACG | NO: 94 |
| | | TTTTT | |
| | | ATACAACCCTCAACTTTACC | |

TABLE 5
Capture DNA oligonucleotides corresponding
to MS2 probes and the DNA carrier comple-
mentary oligonucleotides that are replaced
therewith to form the MS2 DNA carrier. The
region of the capture oligonucleotide com-
plementary to the corresponding target
probe is in bold.

| SEQ | | | Oligonu- |
|---|---|---|---|
| ID | | | cleotide |
| NO | Code | Capture oligo (5′-3′) | replaced |
| 27 | cM1_42 | TTCGACAACTCGTATTAAATCC | SEQ ID |
| | | TTTGCCCGAACGTTATTTTTT | NO: 42 |
| | | GGATATCACTCATTAGTGGT | |
| 28 | cM2_55 | GTGAGTGAATAACCTTGCTTCT | SEQ ID |
| | | GTAAATCGTCGCTATTTTTTT | NO: 55 |
| | | GAGCATGTTACCTACAGGTA | |
| 29 | cM3_68 | AGAATATAAAGTACCGACAAAA | SEQ ID |
| | | GGTAAAGTAATTCTGTTTTTT | NO: 68 |
| | | GAGATGGAATCGGATGCAGA | |

TABLE 6
Complementary DNA oligonucleotides replaced
to make references (R1 and R2).

| SEQ | | | Oligo |
|---|---|---|---|
| ID | | | re- |
| NO | Name | Sequence (5′→>3′) | placed |
| 30 | Reference | ACATCACTTGCCTGAGTAGA | 26-30 |
| | 1 (R1) | AGAACTCAAATCCTCTTTTGAGGA | |
| | | ACAAGTTTTCTTGTCTATCGGCCT | |
| | | TGCTGGTAATTCCTCTTTTGAGGA | |
| | | ACAAGTTTTCTTGTATCCAGAACA | |
| | | ATATTACCGCTCCTCTTTTGAGGA | |
| | | ACAAGTTTTCTTGTCAGCCATTGC | |
| | | AACAGGAAAATCCTCTTTTGAGGA | |
| | | ACAAGTTTTCTTGTACGCTCATGG | |
| | | AAATACCTACTCCTCTTTTGAGGA | |
| | | ACAAGTTTTCTTGTATTTTGACGC | |
| | | TCAATCGTCTTCCTCTTTTGAGGA | |
| | | ACAAGTTTTCTTGTGAAATGGATT | |
| | | ATTTACATTGGCAGATTCAC | |
| | | CAGTCACACGACCAGTAATAAAAG | |
| | | GGACAT | |
| 31 | Reference | TCACAAACAAATAAATCCTCATTA | 106-112 |
| | 2 (R2) | AAGCCAGAATGGAAAGCGCAGTCT | |
| | | CTGAATTTACCGTTCCAGTAAGCG | |
| | | TCAT | |
| | | ACATGGCTTTTCCTCTTTTGAGGA | |
| | | ACAAGTTTTCTTGTTGATGATACA | |
| | | GGAGTGTACTTCCTCTTTTGAGGA | |
| | | ACAAGTTTTCTTGTGGTAATAAGT | |
| | | TTTAACGGGGTCCTCTTTTGAGGA | |
| | | ACAAGTTTTCTTGTTCAGTGCCTT | |
| | | GAGTAACAGTTCCTCTTTTGAGGA | |
| | | ACAAGTTTTCTTGTGCCCGTATAA | |
| | | ACAGTTAATGTCCTCTTTTGAGGA | |
| | | ACAAGTTTTCTTGTCCCCCTGCCT | |
| | | ATTTCGGAACTCCTCTTTTGAGGA | |
| | | ACAAGTTTTCTTGTCTATTATTCT | |
| | | GAAACATGAAAGTATTAAGA | |
| | | GGCTGAGACTCCTCAAGAGAAGGA | |
| | | TTAGGATTAGCGGGGTTTTGCTCA | |
| | | GT | |

Signalling oligonucleotides were also added to the DNA carrier assembly mixture. Signalling oligonucleotides correspond to the target probe but lack the first six nucleotides at the 5′ end and the 3′ end comprises a biotin label. The signalling oligonucleotide binds to the complementary region of the capture oligonucleotide to generate capture-signalling hybrids with a 3′ toehold (i.e. overhang) that serves as a seed for the strand displacement reaction and target probe detection.

The biotinylated signalling oligonucleotides which are partially complementary to the overhang region of the capture oligonucleotides listed in Tables 4 and 5 are listed in Tables 7 and 8, respectively.

TABLE 7
Biotinylated signalling DNA oligonucleotides complementary to SARS-CoV-2 probes.

| SEQ ID | | | |
|---|---|---|---|
| NO | Code | Biotin strand to be displaced (5′-3′) | Length (nt) |
| 32 | B1 | TGAAGAAGAAGAGT/3′-biotin | 14 |
| 33 | B2 | GGAGCTAAATTGTT/3′-biotin | 14 |
| 34 | B3 | GATTTTTGTGGAAA/3′-biotin | 14 |
| 35 | B4 | TTATTCTGTTATTT/3′-biotin | 14 |
| 36 | B5 | GTTGAGGGTTGTAT/3′-biotin | 14 |

TABLE 8
Biotinylated signalling DNA oligonucleotides complementary to MS2 probes.

| SEQ ID | | | |
|---|---|---|---|
| NO | Code | Biotin strand to be displaced (5′-3′) | Length (nt) |
| 37 | B1_MS2 | AATGAGTGATATCC/3′-biotin | 14 |
| 38 | B2_MS2 | TAGGTAACATGCTC/3′-biotin | 14 |
| 39 | B3_MS2 | TCCGATTCCATCTC/3′-biotin | 14 |

After preparation of the linear DNA scaffold and the oligonucleotide sets, the DNA carrier was assembled by mixing, heating up and cooling down. First, the solutions were prepared as following: 8 μL cut M13mp18 DNA scaffold (100 nM); 12 μL oligonucleotide mix (each oligo 200 nM, oligos after phosphorylation); 6 μL 100 mM MgCl₂; 2.9 μL 100 mM Tris-HCl (pH=8.0), 10 mM EDTA; 11.1 μL Mili-Q water. The mixture was heated to 70° C. for 30 s followed by a linear cooling ramp to 25° C. over 50 minutes.

The annealed DNA carrier sample is then purified using a 100 kDa Amicon filter by mixing the annealed mixture with 460 μL washing buffer (10 mM Tris-HCl (pH=8.0), 0.5 mM MgCl₂) and centrifugated at 9,000×G for 10 minutes. This step was repeated twice. The purified DNA carrier was retrieved by spinning for 2 minutes at 1,000×G.

### MS2 Probe Production

The MS2 probes were cut from ˜3.6 kb MS2 RNA. Firstly, cutting oligonucleotides adjusted to the target probes were annealed to target RNA by mixing the cutting oligonucleotides listed in Table 4 at a concentration of 1 μM in 1×RNase H reaction buffer and with 330 nM of target RNA. The mixture was heated to 65° C. for 5 minutes, then incubated at 21° C. for 5 minutes and immediately transferred to ice.

In the next step, 1.5 units of RNase H was added to mixture. The mix was incubated for 20 minutes at 37° C. to allow RNase H to cut RNA strands in the RNA:DNA hybrid regions where cutting oligonucleotides were bound to the target RNA. The enzyme was temperature-inactivated at 65° C. for 20 minutes.

### Capture-Probe Hybrid Production

The strand displacement reaction (SDR) was performed in a buffer that contains 10 mM MgCl₂ and 100 mM NaCl with DNA carrier concentration of 6.72 nM and different excess of target probes with known concentration or ten times excess of expected probe concentration after RNase H cutting of MS2 RNA for 10 minutes at room temperature.

### Native Agarose Gel Electrophoresis

All samples were run on a 1% (w/v) agarose gel prepared in fresh 1×TBE buffer with autoclaved Milli-Q water for 180 minutes, at 70V on ice. The RNA sample was loaded on the gel and fresh 1×purple loading dye without SDS (NEB) or 1× orange dye was used. The gel was poststained in 1×GelRed buffer (Biotium) for 10 minutes and imaged with a GelDoc-It™ (UVP).

### Nanopore Measurement and Data Analysis

The presence or absence of target probes was determined using nanopore detection. 14±3 nm glass nanopores were fabricated by pulling quartz capillaries with filaments (0.5 mm outer diameter and 0.2 inner diameters, Sutter Instrument, USA) using a laser-assisted puller (P-2000, Sutter Instruments). DNA carriers were diluted to 0.3-1 nM concentration in 4M LiCl (pH 9.4) with 9 times excess of monovalent streptavidin to each biotin site on DNA carrier and measured using Axopatch 200B current amplifier at the constant voltage of 600 mV. The same filters and the nanopore setup were used as described in Bell, N. A. W. & Keyser, U. F. Nat. Nanotechnol. 11, 645-651 (2016).

Data analysis of nanopore current traces for each sample was performed using LabView algorithms. Briefly, single DNA events were extracted by setting a current drop threshold to 0.8 nA and minimal duration of 0.5 ms and the event charge deficit (ECD) i.e. event area in the range from 50 to 1000 fC. The characteristics of events were determined using the procedure described in Bell, N. A. W. & Keyser, U. F. Nat. Nanotechnol. 11, 645-651 (2016).

### Results and Discussion

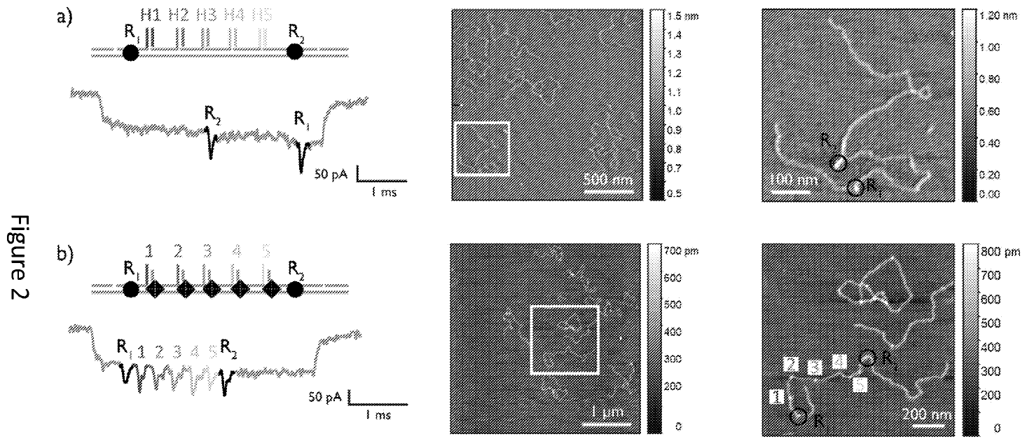

Firstly, the structure of the DNA carrier with and without streptavidin-biotin complex was validated. The DNA carrier comprises two labels that are used to identify the location of the capture oligonucleotides. In the positive control (FIG. 2A), there are two downward peaks in the DNA carrier current trace originating from reference labels in the DNA carrier. The absence of peaks in locations corresponding to the locations of the capture oligonucleotides indicates absence of capture-signalling hybrids, and therefore presence of target probe. In the negative control (FIG. 2B), there are seven downward peaks in the DNA carrier current trace, two originate from reference peaks in the DNA carrier and the middle five peaks correspond to biotin-streptavidin complexes at five capture oligonucleotide (i.e. target probe binding) sites. These five peaks indicate presence of capture-signalling hybrids, and therefore absence of target probe. This is also observed with AFM imaging as shown in FIG. 2.

The displacement of signalling oligonucleotide by target probes (i.e. strand displacement reaction (SDR)) was validated with synthetic SARS-COV-2 RNA and DNA oligonucleotides using nanopore readout. The effect of the probe to DNA carrier ratio on the level of the SDR was assessed for ratios from 0 to 10 times. The SDR mix was incubated for 10 minutes. The occupied fraction refers to the absolute number of detected downward peaks between two references. An occupied fraction of 1 means that the probe is not present, and that the signalling oligonucleotide is not displaced, and an occupied fraction of 0 means that the probe is present, and, in all events, the signalling oligonucleotide is displaced.

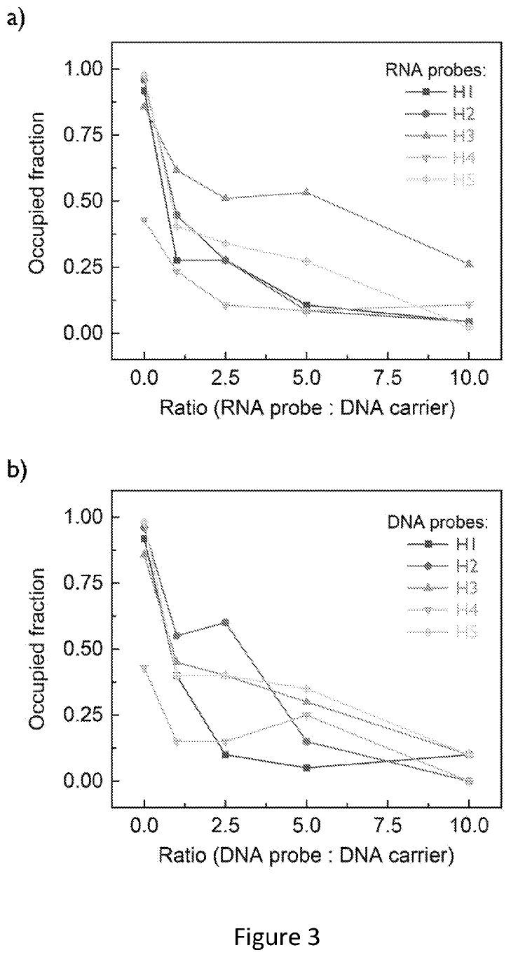

The data for RNA and DNA 20 nt target probes are plotted in FIG. 3A and FIG. 3B, respectively. From both data sets, it can be concluded that 1:10 probe to DNA carrier ratio is enough for efficient SDR. However, even in 1:1 ratio a certain portion of events have displaced sites. The level of the SDR varies from site to site and depends on the nature of target probe (RNA or DNA). These differences can be ascribed to the toehold sequence since it is shown that higher CG content improves the SDR. Surprisingly, RNA probes are not always more efficient in the SDR despite the higher stability of DNA: RNA hybrids compared to DNA: DNA duplexes.

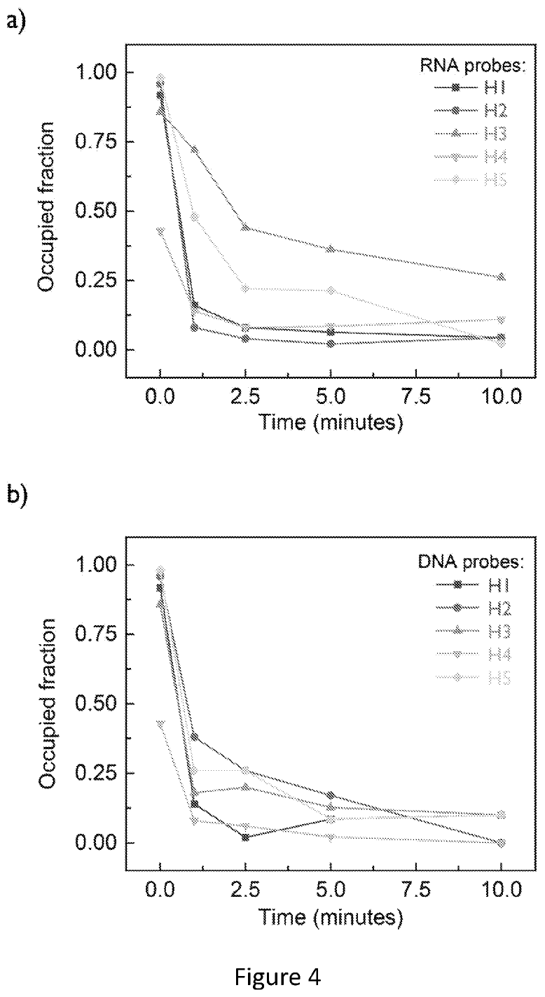

The following experiments were performed with constant 10:1 probe to DNA carrier ratio with different incubation times for the SDR. Here, RNA probes (FIG. 4A) are slower in the displacement of the probe H3 but faster for probe H2 than DNA probes of the same sequence (FIG. 4B). These results can be ascribed to lower or higher RNA:DNA hybrid stability compared to DNA: DNA duplex stability that relates to secondary structure and sequence.

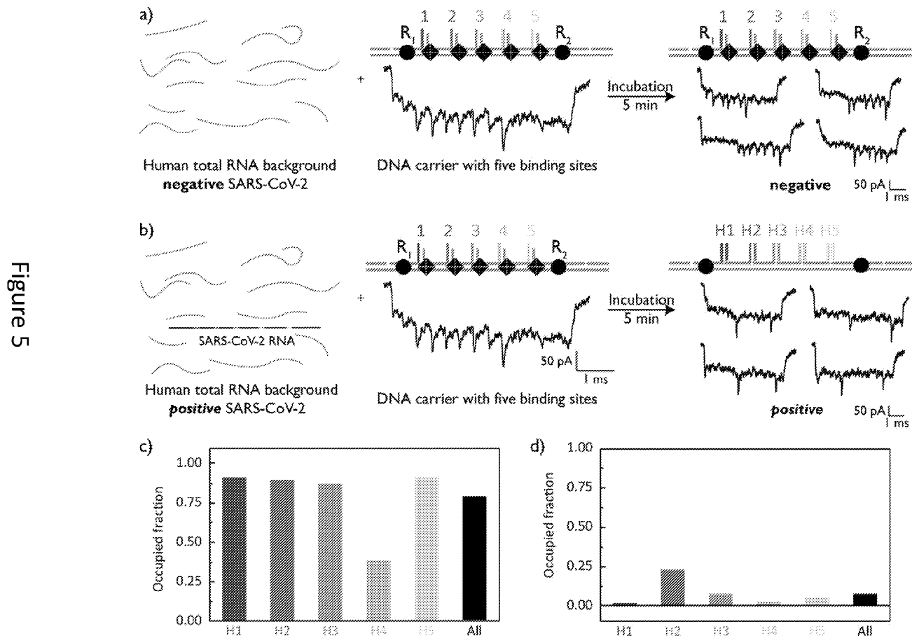

The specificity of the DNA carrier for target probes was then verified in the complex mixture of human total RNA. For the negative control, the level of the SDR did not significantly change with incubation with 300 ng of human total RNA with ^{˜}2.4 ng of DNA carrier under the same SDR conditions (FIG. 5A). In the positive control, target probes successfully displaced all five sites as shown in FIG. 5B. The occupied fraction for each site for the negative and the positive controls is shown in FIG. 5C and FIG. 5D, respectively.

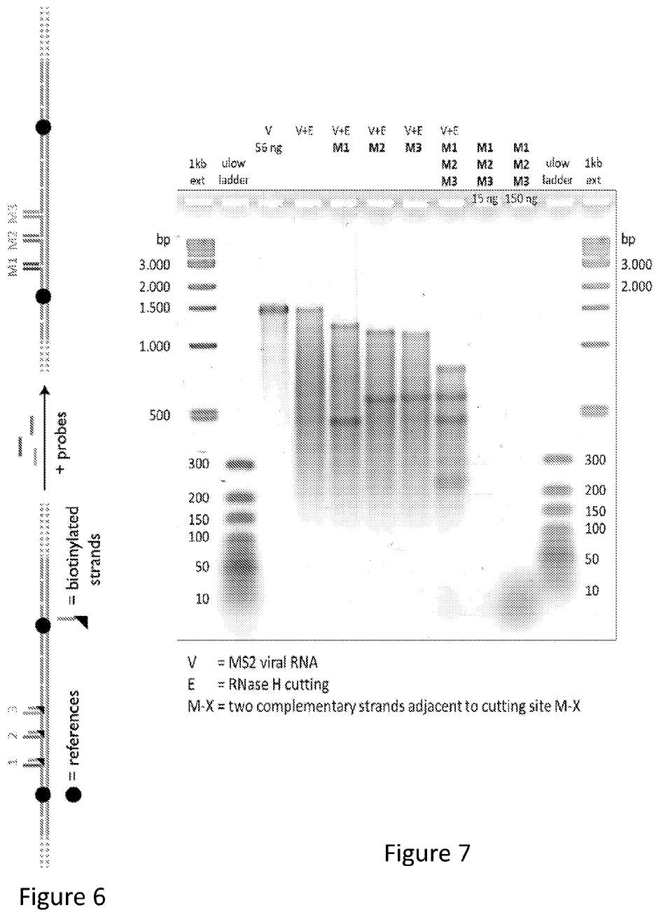

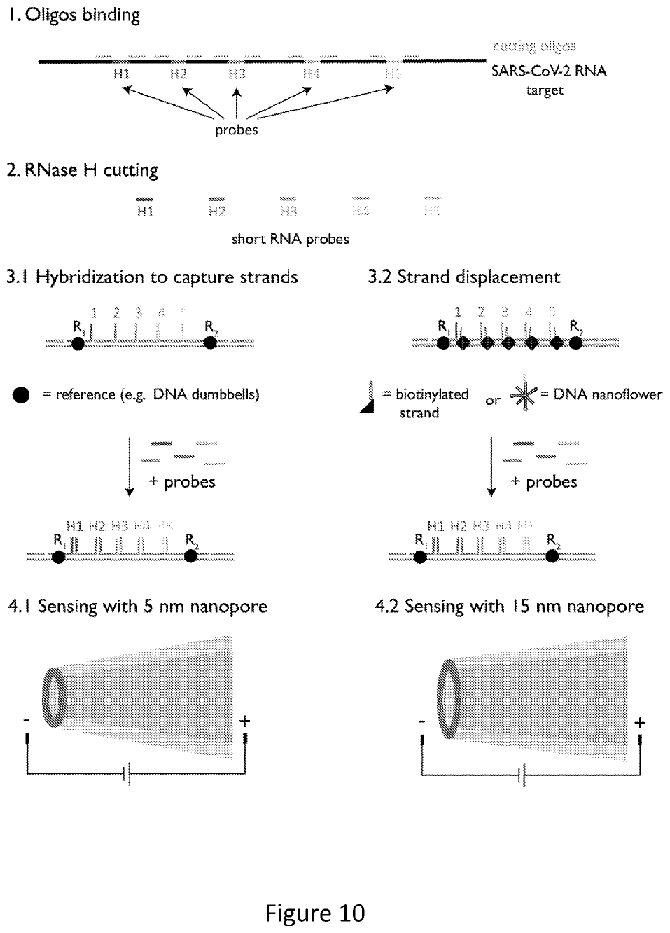

RNase H cutting of the viral RNA was optimized with MS2 RNA phage by designing three probes (M1-M3) to be cut from RNA and with corresponding three sites on the DNA carrier (FIG. 6). Example events (i.e. current traces/signatures) of the DNA carrier with all three sites with biotin-streptavidin complex are shown in FIG. 9A.

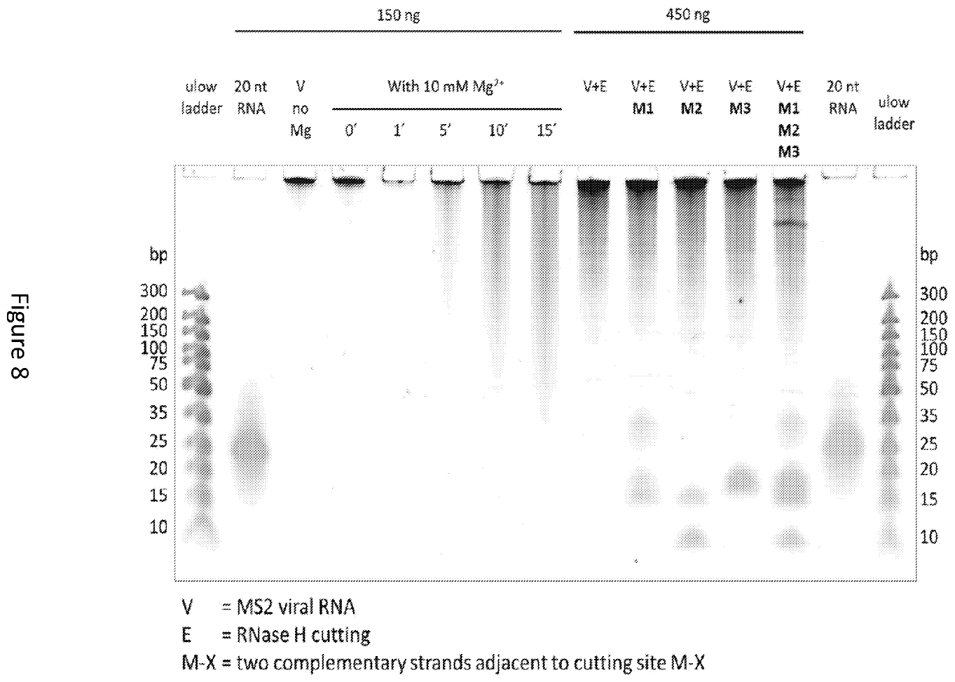

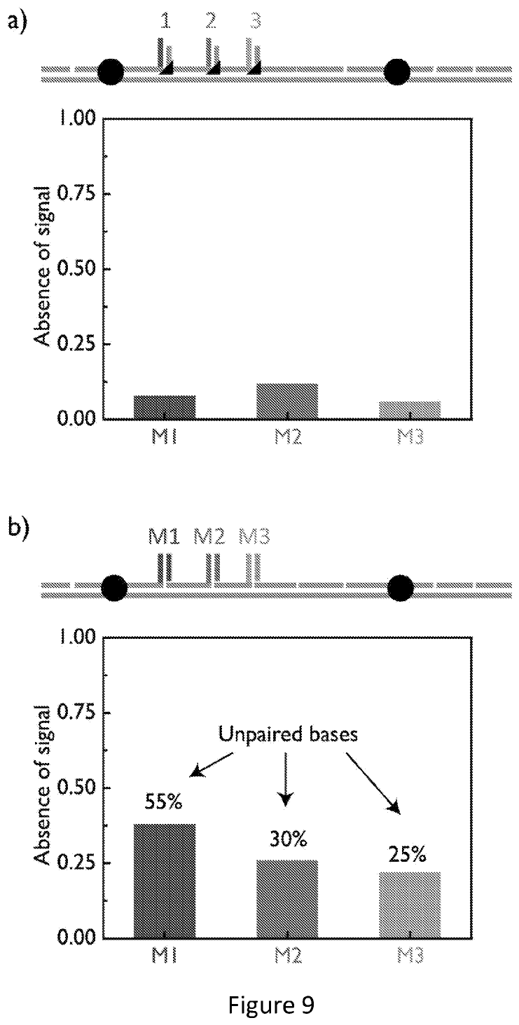

The efficiency of RNase H cutting was estimated with native 2% (w/v) agarose gel electrophoresis as illustrated in FIG. 7. Firstly, the cutting efficiency was verified for individual sites M1, M2 and M3 (V+E, M1/M2/M3) and the fragment with expected length can be observed after cutting. Multisite cutting was also verified to be successful (V+E, M1, M2, M3). The length of probes that is of great importance for SDR was verified by higher resolution denaturing PAGE (FIG. 8).

Methods known in the art for randomly fragmenting RNA include, for example, incubating RNA in the presence of MgCl₂ at high temperature. However, incubation of MS2 RNA in 10 mM MgCl₂ at 94° C. for 15 minutes was less efficient at fragmenting MS2 RNA to produce short, e.g. 20 nt, probes (FIG. 8). These results indicate that the methods of the invention achieve more efficient probe production than methods involving random fragmentation.

The SDR with cut MS2 probes M1-M5 (see Table 2) were tested with the DNA carrier and nanopores. In ten times excess of MS2 RNA after cutting, displacement of sites varied according to the position of each probe in 3D RNA structure. The blank control (no probes added) has all five sites occupied as indicated in FIG. 9A. The probe displacement varies according to the position of probe and likelihood of being unstructured (FIG. 9B). It may be the case that the probe is complementary to another region of RNA meaning the probe stays in dsRNA form and cannot perform stand displacement after cutting resulting in a lower effective concentration than expected. The position of the probe in the 3D RNA structure, as well as the probe length, can be taken into account when optimising probe design.

Based on the results described herein, the inventors identified the following criteria for the optimisation of target probes: (a) a GC content of 40-60%; (b) a terminal region that has a GC content of 40-60%; (c) a high specificity, i.e. low similarity to other nucleic acids that may be present in the sample to limit cross-hybridization; and/or (d) located in unstructured i.e. unhybridized regions of the target nucleic acid.

### Example 2

### Detection of SARS-COV-2 in Patient Swab Samples

The method of the invention was used to detect the presence or absence of SARS-COV-2 in the presence of total nucleic acid isolated from a patient swab sample. The method step are as follows:

### 1. Recognition of Target Probes:

Reaction mixture: 8 μL patient sample (positive or negative); 1 μL 10×RNase H buffer (contains MgCl₂ and KCl); 0.6 μL of cutting oligo mix (16.67 UM of each cutting oligo)
Binding: 5 minutes at 70° C., 25° C. for minutes and transferred immediately to ice

### 2. Excision of Target Probes by RNase H Cutting:

0.3 μL RNase H added to reaction mixture
Cutting: 20 minutes at 37° C.
Inactivation of RNase H: 20 minutes at 65° C.
   3. Contacting target probe with capture oligonucleotides:
Reaction mixture: 5 μL mix of cut sample; 4 μL of DNA carrier (final concentration ^{˜}3 nM); 0.5 μL 100 mM MgCl₂; 0.5 μL 1M NaCl
Incubated at room temperature

### 4. Detection of Capture-Signalling Hybrids by Nanopore:

Reaction mixture: 7 μL 4 M LiCl (pH=9.39); 4 μL 8M LiCl; 1.6 μL SDR mix; 2.4 μL monovalent streptavidin (200 nM)
Incubated for 5 min

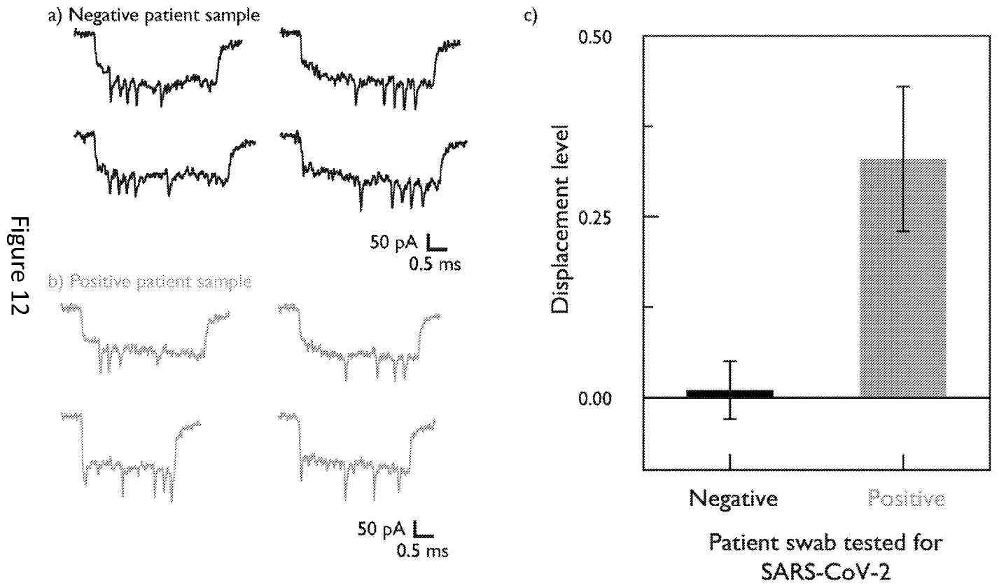

Results on the detection of SARS-COV-2 sequences in the presence of human total RNA background are shown in FIG. 11. Five distinct probes in the SARS-COV-2 RNA genome were identified. In the negative control (absence of target probe) the DNA carrier current trace remains unchanged relative to the pre-incubation current trace, displaying 7 peaks indicating that the sample is negative for the virus. In the positive control, the DNA carrier current trace shows only two peaks signalling the absence of the biotin/streptavidin labelled capture-signalling hybrids and indicating the presence of SARS-COV-2 in the sample. All measurements were performed in a background of human total RNA background at a concentration of 10 ng/μL.

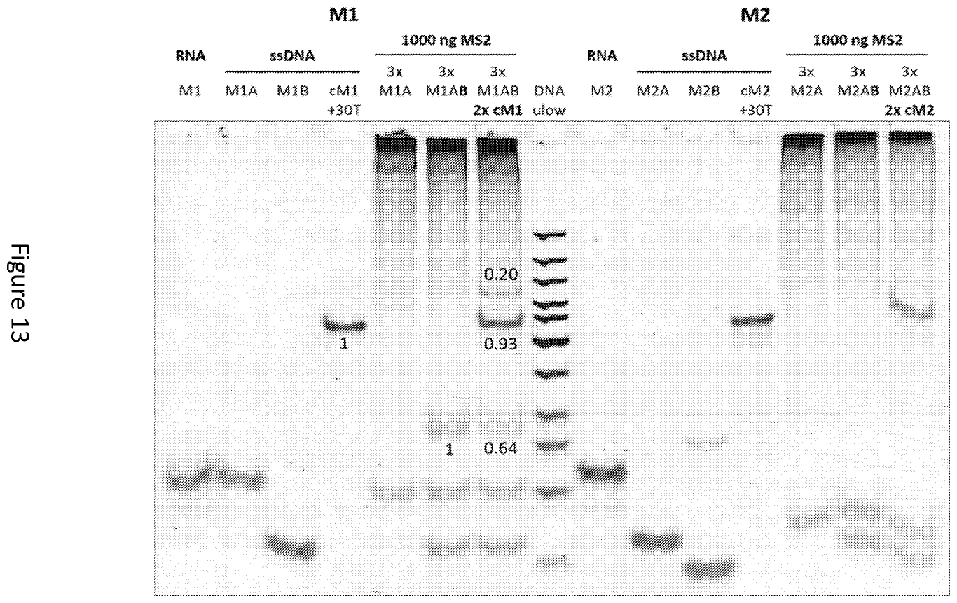

Human patient swab samples that had previously been tested using RT-PCR were incubated with capture oligonucleotides for 10-30 minutes (for positive and negative samples). The results are shown in FIG. 12 (negative sample shown in black; positive sample shown in grey). The displacement level for the positive sample is significantly higher than the negative sample for the three probes used in the study.

### Example 3

Short RNA Probe Cleavage from Long RNA

To test the hypothesis that the position of a probe in a folded RNA affects the free concentration of that probe in the solution, the inventors used MS2 RNA (10165948001 Roche). MS2 RNA is single-stranded and 3569 nucleotide long. The inventors designed multiple short probes based on their position in folded RNA (Dai et al. Nature. 2017; 541 (7635): 112-116, and The Vienna RNA Web suite). Two example probes, M1 and M2, were designed to be in the unpaired RNA region (M1) or the paired RNA region (M2) of MS2.

The probe cutting process is described in Example 1 and includes: annealing cutting DNA oligonucleotides MXA and MXB (where X is 1 or 2) to MS2 RNA; RNaseH cutting of the DNA: RNA hybrid sites; and validating data using polyacrylamide gel electrophoresis (PAGE) and nanopore detection.

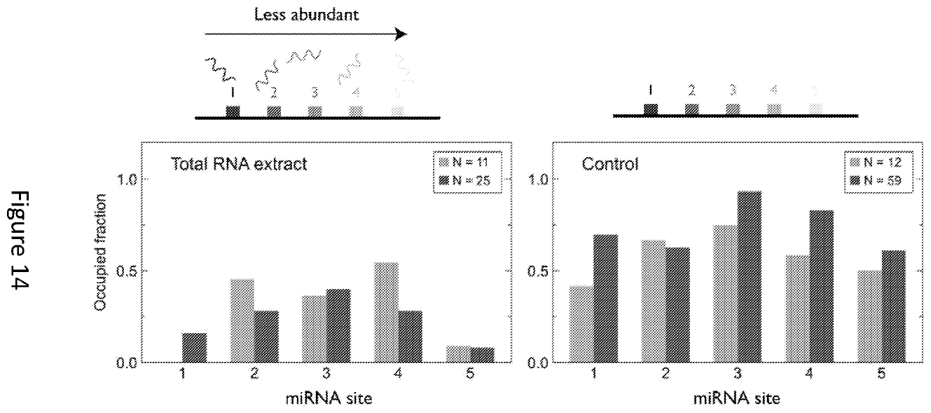

The results of cutting for these two probes are shown in FIG. 13. For M1 the inventors used adjusted 20 nt long cutting oligonucleotides M1A and M1B to extract a 20 nt long RNA probe. Cutting MS2 RNA with only one cutting oligonucleotide (M1A) produces a band corresponding the M1A cutting oligonucleotide only (FIG. 13, 3×M1A lane). When both cutting oligonucleotides (M1A and M1B) are used, two lower bands that correspond to the cutting oligonucleotides and an additional band that corresponds to the ^{˜}20 nt long probe (M1) are detected (FIG. 13, 3×M1AB lane).

To confirm that the additional band corresponds to the target probe, the inventors added oligonucleotide cM1 (which has a 30 nt tail) which is complementary to the target probe. In the presence of cM1, the gel shift confirms that the additional band indeed corresponds to free target probe M1 (FIG. 13, 3×M1AB 2×cM1 lane).

The same experiments were repeated for the M2 probe (FIG. 13, M2 lanes). No additional band that would correspond to the free target probe M2 in solution was identified (FIG. 13, 3×M2AB lane) indicating that this probe has not been efficiently excised from the MS2 target.

These results indicate that target probes positioned in an unpaired region (e.g. M1) are more efficiently cleaved and released in solution than target probes positioned in a paired region (e.g. M2). Cleavage and release of target probes into solution is essential for successful detection of target probes by toehold-mediated SDR.

Table 9 demonstrates the occupied fractions for DNA carrier comprising five detection sites for five MS2 20 nt probes (M1-M5) in the presence of different samples. Sample 1 (blank control) indicates that in the absence of probes the occupied fraction is high. Sample 2 (probes M4 and M5 present) and sample 3 (probes M1, M4 and M5 present) indicate a reduced occupied fraction for sites corresponding to the probes present in the sample, indicating detection of these probes.

TABLE 9
Occupied fraction for each site without (Sample 1) and with
M4 and M5 oligonucleotides (Sample 2). Sample 3 shows
occupied fractions when the cut MS2 RNA for sites 1, 4,
and 5 are used.

| SAMPLE | TARGET ADDED | M1 | M2 | M3 | M4 | M5 |
|---|---|---|---|---|---|---|
| 1 | Blank control | 0.90 | 0.88 | 0.92 | 0.83 | 0.54 |
| 2 | M4, M5 DNA | 0.84 | 0.95 | 0.88 | 0.031 | 0.063 |
| 3 | M1, M4, M5 | 0.28 | 0.90 | 0.85 | 0.090 | 0.12 |

RNA Probe Cleavage from SARS-COV-2 RNA Isolated from a Patient Sample

Using the same protocol as for MS2 RNA targets the inventors designed three probes (S1, S2, and S3) present in the conserved domain of SARS-COV-2 RNA and predicted to be partially unpaired.

Table 10 demonstrates the occupied fractions for: sample 1 is a negative control with a negative patient swab sample; sample 2 is a positive control with S1, S2 and S3 20 nt probes added instead of patient sample in the estimated similar concentration with the negative patient swab sample 1; and sample 3 is a positive sample with probes S1, S2 and S3 cut from a positive patient samples.

TABLE 10
Occupied fraction for each site using the same protocol with negative
sample (Sample 1) and with S1, S2, and S3 oligonucleotides
(Sample 2). Sample 3 shows data when the cut SARS-COV-2 RNA
for sites 1, 2, and 3 are used in positive patient sample background.

| SAMPLE | TARGET ADDED | S1 | S2 | S3 |
|---|---|---|---|---|
| 1 | Blank control | 1 | 1 | 1 |
| | (negative patient sample) | | | |
| 2 | S1, S2, S3 DNA | 0.67 | 0.5 | 0.34 |
| 3 | S1, S2, S3 cut from SARS- | 0.67 | 0.67 | 0.5 |
| | CoV-2 RNA | | | |
| | (positive patient sample) | | | |

TABLE 11
S1-S3 SARS-CoV-2 probe cutting DNA oligonucleotides.

| SEQ ID | | | |
|---|---|---|---|
| NO | Code | Cutting oligo (5′-3′) | Length (nt) |
| 40 | S1A | GCTAGTGTAACTAGCAAGAA | 20 |
| 41 | S1B | ATTGCAGCAGTACGCACACA | 20 |
| 42 | S2A | CGTTCTCCATTCTGGTTACT | 20 |
| 43 | S2B | GTGATCTTTTGGTGTATTCA | 20 |
| 44 | S3A | GATAACTAGCGCATATACCT | 20 |
| 45 | S3B | GCTACACTACGTGCCCGCCG | 20 |

TABLE 12
Target probe sequences, with length of 20 nt, in the SARS-CoV-2 RNA genome.

| SEQ ID | | | |
|---|---|---|---|
| NO | Code | Target RNA/DNA probe (5′-3′) | Length (nt) |
| 46 | S1 | CATCCTTACTGCGCTTCGAT | 20 |
| 47 | S2 | CATTGCAACTGAGGGAGCCT | 20 |
| 48 | S3 | AGACTCAGACTAATTCTCCT | 20 |

TABLE 13
Biotinylated signalling DNA oligonucleotides for the toehold-mediated strand
displacement reaction for the SARS-CoV-2 DNA carrier.

| SEQ ID | | | |
|---|---|---|---|
| NO | Code | Biotin strand to be displaced (5′-3′) | Length (nt) |
| 49 | bS1 | TACTGCGCTTCGAT/3′-biotin | 14 |
| 50 | bS2 | AACTGAGGGAGCCT/3′-biotin | 14 |
| 51 | bS3 | AGACTAATTCTCCT/3′-biotin | 14 |

TABLE 14
Capture DNA oligonucleotides and the complementary DNA oligonucleotides (table 22) they
replace to produce the MS2 DNA carrier.

| SEQ ID | | | Oligonucleotide |
|---|---|---|---|
| NO | Code | Capture oligo (5′-3′) | replaced |
| 52 | cS1_42 | TTCGACAACTCGTATTAAATCCTTTGCCCGAACGTTAT TTTTT | SEQ ID NO: 42 |
| | | ATCGAAGCGCAGTAAGGATG | |
| 53 | cS2_55 | GTGAGTGAATAACCTTGCTTCTGTAAATCGTCGCTATT TTTTT | SEQ ID NO: 55 |
| | | AGGCTCCCTCAGTTGCAATG | |
| 54 | cS3_68 | AGAATATAAAGTACCGACAAAAGGTAAAGTAATTCTGT TTTTT | SEQ ID NO: 68 |
| | | AGGAGAATTAGTCTGAGTCT | |

### Example 4

C. elegans miRNA Detection

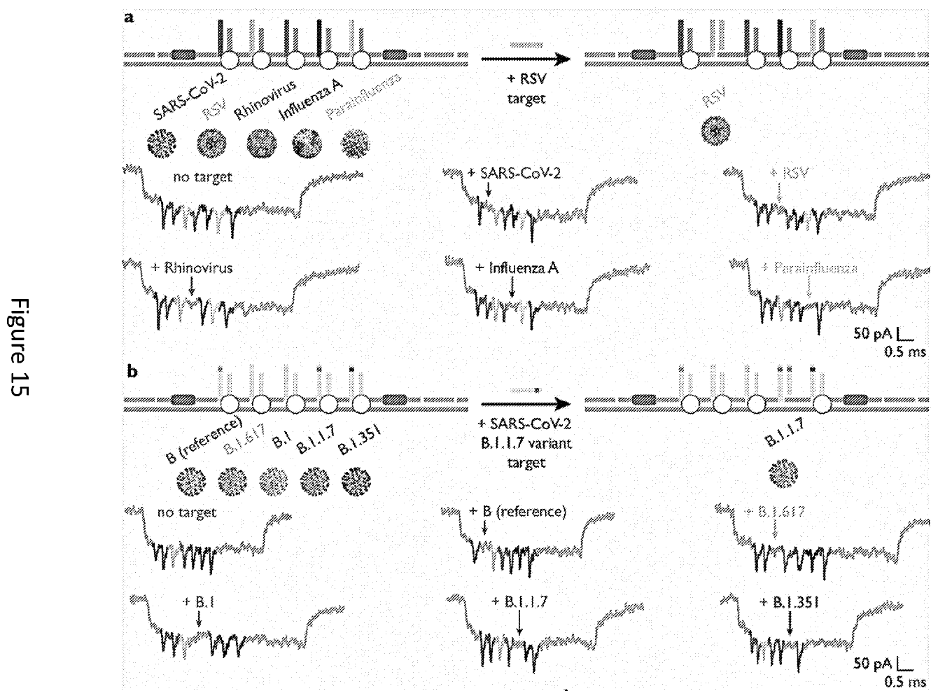

miRNAs play an essential role in the development of C. elegans and it is described in the literature that more than one miRNA is necessary to influence development. The method of the invention was used to detect miRNAs in C. elegans. Five miRNAs (miR-58, miR-1, miR-71, miR-70, miR-72) that have previously been described to play a role in the development of C. elegans (Kato, M. et al. Genome Biol 10, R54 (2009)) were analysed using the methods of the invention. Results on the detection of the miRNA sequences in the presence of C. elegans total RNA background are shown in FIG. 14 (1, 2, 3, 4, and 5 correspond to miR-58, miR-1, miR-71, miR-70, miR-72, respectively). The SDR had 0.5 μl total RNA extract (200-400 ng/μl), 1.0 μl DNA carrier (10 nM), 0.5 μl NaCl (1 M), 0.5 μl MgCl₂ (100 mM), 2.5 μl H₂O. Nanopore measurements were performed by mixing 5 μl of the SDR sample, 5 μl LiCl (8 M), and 10 μl LiCl (4 M) while ˜15 μl was added into nanopore chip.

### Example 5

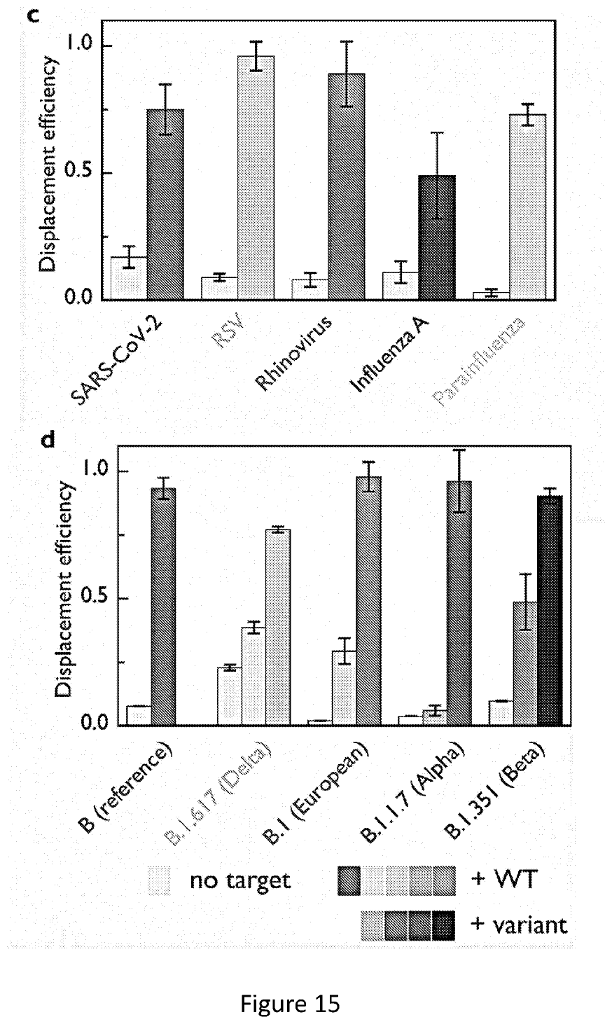

Simultaneous identification of multiple respiratory viruses and SARS-COV-2 single-nucleotide variants Simultaneous detection of multiple viruses is of great clinical relevance for respiratory virus diagnostics. Firstly, respiratory viruses can have a similar clinical manifestation and co-infection with multiple viruses are common and increase the severity and mortality of respiratory diseases. The inventors employed a DNA carrier for multiplexed target identification of five different respiratory viruses including SARS-COV-2, influenza A type, Respiratory Syncytial Virus (RSV), parainfluenza A type, and rhinoviruses (sequences are listed in Tables 15-17, and design principles are provided below). The design of DNA carriers for multiple respiratory viruses/viral groups is shown in FIG. 15a. In the absence of any target nucleic acid/probes, all peaks corresponding to capture-signalling hybrids are present. In the presence of the respective target probes for SARS-COV-2, influenza A type, RSV, parainfluenza, and rhinoviruses, that peak is absent in DNA carrier nanopore events (FIG. 15a). Displacement efficiency for the first fifty linear DNA carrier events for each sample on at least three different nanopores is plotted in FIG. 15c. These results indicate that in the presence of specific target, the corresponding peak (signalling oligo) is displaced/absent.

The ability to detect and track viral variants is important for both clinical decision making and epidemiology. A particular difficulty when detecting viral variants is that they may deviate from the wild-type virus by only a single nucleotide variation. To confirm that the methods of the invention could detect and differentiate between single nucleotide variants (SNVs), the inventors designed DNA carriers to detect emerging SARS-COV-2 variants (sequences are listed in Tables 18-21, and design principles are provided below). The DNA carrier has five capture oligonucleotide sites specific to a reference strain first isolated in Wuhan (B by PANGOLIN nomenclature); European strain B.1; and three variants of the European strain defined as variants of concern B.1.1.7 (alpha), and B.1.351 (beta), and B.1.617 (delta) first detected in United Kingdom, South Africa, and India, respectively (see FIG. 15b). In the absence of target nucleic acid/probe, all peaks corresponding to capture-signalling hybrids are present. In the presence of each respective target probes for the reference or each variant, the corresponding peak (produced by capture-signalling hybrids) is absent in example nanopore events. (FIG. 15b). Variant-specific target probes are fully complementary to capture oligonucleotides on the DNA carrier, while the wild type (WT) target probe has a mismatch as indicated in Table 20. As demonstrated, the method of the invention can discriminate SARS-COV-2 variants from the WT sequence even in the presence of only a single-nucleotide variation. In FIG. 15d, the inventors plotted the displacement levels for the first fifty DNA carrier events when no targets (control measurement), WT target and variant target are present.

### DNA Carrier Sequences

DNA carrier for multiple respiratory viruses is prepared as described above. Capture oligo sequences (Table 17), biotin signalling strand sequences (Table 16), and target probe sequences (Table 15) for each virus are provided below. Target probes for Influenza A, Parainfluenza A, and Rhinoviruses are common to a group of these viruses rather than a single variant.

DNA carrier for multiple SARS-COV-2 variants is prepared as described above. Capture oligo sequences (Table 21), biotin signalling strand sequences (Table 19), and target probes sequences for wildtype (Table 20) and variants (Table 18) are provided below.

TABLE 15
Target DNA probe sequences for the multiple virus identification.

| SEQ ID | Strand | Virus/group | | |
|---|---|---|---|---|
| NO | name | of viruses | Sequence reference | Sequence (5′→3′) |
| 55 | SW | SARS-CoV-2_Wuhan | NCBI Reference Sequence: | GTATGAAAATGCCTTTTTAC |
| | | | NC_045512.2; Wu, F. et al. | |
| | | | Nature 579:265-269 | |
| | | | (2020) | |
| 56 | Infl | Influenza A | Sequence adapted from | TGACAGGATTGGTCTTGTCT |
| | | viruses | ″WHO information for the | |
| | | universal | molecular detection of | |
| | | | influenza viruses″ July | |
| | | | 2017 | |
| | | | (https://www.who.int/ | |
| | | | influenza/gisrs_laboratory/ | |
| | | | WHO_information_for_the_ | |
| | | | molecular_detection_of_ | |
| | | | influenza_viruses_20171023_ | |
| | | | Final.pdf) | |
| 57 | RSV | Respiratory | Sequence adapted from | ACACAGCAGCTGTTCAGTAC |
| | | syncytial | Shirato, K. et al. J. | |
| | | virus universal | Virological Methods | |
| | | A | 139(1): 78-84 (2007) | |
| 58 | PI | Parainfluenza 1 | Sequence adapted from | CTTCCTGCTGGTGTGTTAAT |
| | | | Templeton, K. E. et al. | |
| | | | Journal of Clinical | |
| | | | Microbiology 42(4): 1564- | |
| | | | 1569 (2004) | |
| 59 | RV | Rhinoviruses | Sequence adapted from | TCCTCCGGCCCCTGAATGTG |
| | | universal | Lu, X. et al. Journal of | |
| | | | Clinical Microbiology | |
| | | | 46(2): 533-539 (2008) | |

TABLE 16
3′ Biotinylated DNA oligo sequences for the multiple virus identification.

| SEQ ID | Strand | Virus/group | |
|---|---|---|---|
| NO | name | of viruses | Sequence (5′→3′) |
| 60 | bSW | SARS-CoV-2_Wuhan | AAATGCCTTTTTAC/3-biotin/ |
| 61 | blnfl | Influenza A viruses | GATTGGTCTTGTCT/3-biotin/ |
| | | universal | |
| 62 | bRSV | Respiratory syncytial | CAGCTGTTCAGTAC/3-biotin/ |
| | | virus universal A | |
| 63 | bPI | Parainfluenza I | GCTGGTGTGTTAAT/3-biotin/ |
| 64 | bRV | Rhinoviruses universal | GGCCCCTGAATGTG/3-biotin/ |

TABLE 17
Capture DNA strand sequences for multiple virus identification.

| SEQ | Strand | Virus/group | |
|---|---|---|---|
| ID NO | name | of viruses | Sequence (5′→3′) |
| 65 | cSW_42 | SARS-CoV-2_Wuhan | TTCGACAACTCGTATTAAATCCTTTGCCCGAACGTTAT |
| | | | TTTTT GTAAAAAGGCATTTTCATAC |
| 66 | cRSV_55 | Respiratory | GTGAGTGAATAACCTTGCTTCTGTAAATCGTCGCTATT |
| | | syncytial virus | TTTTT GTACTGAACAGCTGCTGTGT |
| | | universal A | |
| 67 | CRV_68 | Rhinoviruses | AGAATATAAAGTACCGACAAAAGGTAAAGTAATTCTGT |
| | | universal | TTTTT CACATTCAGGGGCCGGAGGA |
| 68 | cl_81 | Influenza A viruses | TCCCAATCCAAATAAGAAACGATTTTTTGTTTAACGTC |
| | | universal | TTTTT AGACAAGACCAATCCTGTCA |
| 69 | cPl_94 | Parainfluenza 1 | CATTCAACCGATTGAGGGAGGGAAGGTAAATATTGACG |
| | | | TTTTT ATTAACACACCAGCAGGAAG |

TABLE 18
Target sequences in the SARS-CoV-2 genomes for the identification of the multiple variants
defined in the table. Single nucleotide variant positions are highlighted in bold, the
toehold region used for the strand displacement reaction is underlined.

| | | | | Single | |
|---|---|---|---|---|---|
| SEQ ID | Strand | WHO | Pangolin | nucleotide | |
| NO | name | nomenclature | nomenclature | variation | Sequence (5′→3′) |
| 70 | SW | Reference | B (reference) | / | GTATGAAAATGCCTTTTTAC |
| 71 | Slm | Delta | B.1.617 (Indian) | T-G | CCGGTATAGATTGTTTAGGA |
| | | | | L452R | |
| 72 | SEm | NA | B.1 (European) | A-G | GGTGTTAACTGCACAGAAGT |
| | | | | D614G | |
| 73 | SUKm | Alfa | B.1.1.7 (British) | A-T | CTTATGGTGTTGGTTACCAA |
| | | | | N501Y | |
| 74 | SSAm | Beta | B.1.351 (South | G-A | TAAAGGTTTTAATTGTTACT |
| | | | African) | E484K | |

TABLE 19
3′ Biotinylated DNA strand sequences for the multiple SARS-CoV-2 variant identification.

| SEQ ID | | | |
|---|---|---|---|
| NO | Strand name | Variant name | Sequence (5′→3′) |
| 75 | bSW | B (reference) | AAATGCCTTTTTAC/3-BIOTIN/ |
| 76 | bSIm | B.1.617 (Indian) | TAGATTGTTTAGGA/3-BIOTIN/ |
| 77 | bSEm | B.1 (European) | AACTGCACAGAAGT/3-BIOTIN/ |
| 78 | bSUKm | B.1.1.7 (British) | GTGTTGGTTACCAA/3-BIOTIN/ |
| 79 | bSSAm | B.1.351 (South African) | TTTTAATTGTTACT/3-BIOTIN/ |

TABLE 20
DNA sequences for the multiple SARS-CoV-2 variant identification.
WT nucleotide positions are highlighted in bold, the toehold
region used for the strand displacement reaction is underlined.

| SEQ ID NO | Strand name | Sequence (5′→3′) | Length (nt) |
|---|---|---|---|
| 80 | SI | CCTGTATAGATTGTTTAGGA | 20 |
| 81 | SE | GATGTTAACTGCACAGAAGT | 20 |
| 82 | SUK | CTAATGGTGTTGGTTACCAA | 20 |
| 83 | SSA | TGAAGGTTTTAATTGTTACT | 20 |

TABLE 21
Capture DNA strand sequences for the multiple SARS-CoV-2 variant identification.

| SEQ ID | Strand | | |
|---|---|---|---|
| NO | name | Variant name | Sequence (5′→3′) |
| 84 | cSW_42 | B (reference) | TTCGACAACTCGTATTAAATCCTTTGCCCGAACGTTAT TTTTT |
| | | | GTAAAAAGGCATTTTCATAC |
| 85 | cSI_68 | B.1.617 (Indian) | AGAATATAAAGTACCGACAAAAGGTAAAGTAATTCTGT |
| | | | TTTTT TCCTAAACAATCTATACCGG |
| 86 | CSE_94 | B.1 (European) | CATTCAACCGATTGAGGGAGGGAAGGTAAATATTGACG |
| | | | TTTTT ACTTCTGTGCAGTTAACACC |
| 87 | cSUK_81 | B.1.1.7 (British) | TCCCAATCCAAATAAGAAACGATTTTTTGTTTAACGTC TTTTT |
| | | | TTGGTAACCAACACCATAAG |
| 88 | CSSA_55 | B.1.351 | GTGAGTGAATAACCTTGCTTCTGTAAATCGTCGCTATT |
| | | (South African) | TTTTT AGTAACAATTAAAACCTTTA |

TABLE 22
DNA scaffold complementary oligonucleotides

| SEQ | | SEQ | |
|---|---|---|---|
| ID | | ID | |
| NO | Sequence (5′→3′) | NO | Sequence (5′→3′) |
| 89 | TTTTCGTAATCATGGTCATAGCTGTT | 184 | CTTGAGCCATTTGGGAATTAGAGCCAG |
| | TCCTGTGTGAAATTGTTATC | | CAAAATCACCA |
| 90 | CGCTCACAATTCCACACAACATACG | 185 | GTAGCACCATTACCATTAGCAAGGCCG |
| | AGCCGGAAGCATA | | GAAACGTCACC |
| 91 | AAGTGTAAAGCCTGGGGTGCCTAAT | 186 | AATGAAACCATCGATAGCAGCACCGTA |
| | GAGTGAGCTAACT | | ATCAGTAGCGA |
| 92 | CACATTAATTGCGTTGCGCTCACTG | 187 | CAGAATCAAGTTTGCCTTTAGCGTCAG |
| | CCCGCTTTCCAGT | | ACTGTAGCGCG |
| 93 | CGGGAAACCTGTCGTGCCAGCTGC | 188 | TTTTCATCGGCATTTTCGGTCATAGCCC |
| | ATTAATGAATCGGC | | CCTTATTAGC |
| 94 | CAACGCGCGGGGAGAGGCGGTTTG | 189 | GTTTGCCATCTTTTCATAATCAAAATCA |
| | CGTATTGGGCGCCA | | CCGGAACCAG |
| 95 | GGGTGGTTTTTCTTTTCACCAGTGA | 190 | AGCCACCACCGGAACCGCCTCCCTCA |
| | GACGGGCAACAGC | | GAGCCGCCACCC |
| 96 | TGATTGCCCTTCACCGCCTGGCCCT | 191 | TCAGAACCGCCACCCTCAGAGCCACCA |
| | GAGAGAGTTGCAG | | CCCTCAGAGCC |
| 97 | CAAGCGGTCCACGCTGGTTTGCCC | 192 | GCCACCAGAACCACCACCAGAGCCGC |
| | CAGCAGGCGAAAAT | | CGCCAGCATTGA |
| 98 | CCTGTTTGATGGTGGTTCCGAAATC | 193 | CAGGAGGTTGAGGCAGGTCAGACGAT |
| | GGCAAAATCCCTT | | TGGCCTTGATAT |
| 99 | ATAAATCAAAAGAATAGCCCGAGAT | 194 | TCACAAACAAATAAATCCTCATTAAAGC |
| | AGGGTTGAGTGTT | | CAGAATGGAA |
| 100 | GTTCCAGTTTGGAACAAGAGTCCAC | 195 | AGCGCAGTCTCTGAATTTACCGTTCCA |
| | TATTAAAGAACGT | | GTAAGCGTCAT |
| 101 | GGACTCCAACGTCAAAGGGCGAAAA | 196 | ACATGGCTTTTGATGATACAGGAGTGT |
| | ACCGTCTATCAGG | | ACTGGTAATAA |
| 102 | GCGATGGCCCACTACGTGAACCATC | 197 | GTTTTAACGGGGTCAGTGCCTTGAGTA |
| | ACCCAAATCAAGT | | ACAGTGCCCGT |
| 103 | TTTTTGGGGTCGAGGTGCCGTAAAG | 198 | ATAAACAGTTAATGCCCCCTGCCTATTT |
| | CACTAAATCGGAA | | CGGAACCTAT |
| 104 | CCCTAAAGGGAGCCCCCGATTTAGA | 199 | TATTCTGAAACATGAAAGTATTAAGAGG |
| | GCTTGACGGGGAA | | CTGAGACTCC |
| 105 | AGCCGGCGAACGTGGCGAGAAAGG | 200 | TCAAGAGAAGGATTAGGATTAGCGGGG |
| | AAGGGAAGAAAGCG | | TTTTGCTCAGT |
| 106 | AAAGGAGCGGGCGCTAGGGCGCTG | 201 | ACCAGGCGGATAAGTGCCGTCGAGAG |
| | GCAAGTGTAGCGGT | | GGTTGATATAAG |
| 107 | CACGCTGCGCGTAACCACCACACC | 202 | TATAGCCCGGAATAGGTGTATCACCGT |
| | CGCCGCGCTTAATG | | ACTCAGGAGGT |
| 108 | CGCCGCTACAGGGCGCGTACTATG | 203 | TTAGTACCGCCACCCTCAGAACCGCCA |
| | GTTGCTTTGACGAG | | CCCTCAGAACC |
| 109 | CACGTATAACGTGCTTTCCTCGTTA | 204 | GCCACCCTCAGAGCCACCACCCTCATT |
| | GAATCAGAGCGGG | | TTCAGGGATAG |
| 110 | AGCTAAACAGGAGGCCGATTAAAGG | 205 | CAAGCCCAATAGGAACCCATGTACCGT |
| | GATTTTAGACAGG | | AACACTGAGTT |
| 111 | AACGGTACGCCAGAATCCTGAGAAG | 206 | TCGTCACCAGTACAAACTACAACGCCT |
| | TGTTTTTATAATC | | GTAGCATTCCA |
| 112 | AGTGAGGCCACCGAGTAAAAGAGTC | 207 | CAGACAGCCCTCATAGTTAGCGTAACG |
| | TGTCCATCACGCA | | ATCTAAAGTTT |
| 113 | AATTAACCGTTGTAGCAATACTTCTT | 208 | TGTCGTCTTTCCAGACGTTAGTAAATGA |
| | TGATTAGTAATA | | ATTTTCTGTA |
| 114 | ACATCACTTGCCTGAGTAGAAGAAC | 209 | TGGGATTTTGCTAAACAACTTTCAACAG |
| | TCAAACTATCGGC | | TTTCAGCGGA |
| 115 | CTTGCTGGTAATATCCAGAACAATAT | 210 | GTGAGAATAGAAAGGAACAACTAAAGG |
| | TACCGCCAGCCA | | AATTGCGAATA |
| 116 | TTGCAACAGGAAAAACGCTCATGGA | 211 | ATAATTTTTTCACGTTGAAAATCTCCAA |
| | AATACCTACATTT | | AAAAAAGGCT |
| 117 | TGACGCTCAATCGTCTGAAATGGAT | 212 | CCAAAAGGAGCCTTTAATTGTATCGGT |
| | TATTTACATTGGC | | TTATCAGCTTG |
| 118 | AGATTCACCAGTCACACGACCAGTA | 213 | CTTTCGAGGTGAATTTCTTAAACAGCTT |
| | ATAAAAGGGACAT | | GATACCGATA |
| 119 | TCTGGCCAACAGAGATAGAACCCTT | 214 | GTTGCGCCGACAATGACAACAACCATC |
| | CTGACCTGAAAGC | | GCCCACGCATA |
| 120 | GTAAGAATACGTGGCACAGACAATA | 215 | ACCGATATATTCGGTCGCTGAGGCTTG |
| | TTTTTGAATGGCT | | CAGGGAGTTAA |
| 121 | ATTAGTCTTTAATGCGCGAACTGATA | 216 | AGGCCGCTTTTGCGGGATCGTCACCCT |
| | GCCCTAAAACAT | | CAGCAGCGAAA |
| 122 | CGCCATTAAAAATACCGAACGAACC | 217 | GACAGCATCGGAACGAGGGTAGCAAC |
| | ACCAGCAGAAGAT | | GGCTACAGAGGC |
| 123 | AAAACAGAGGTGAGGCGGTCAGTAT | 218 | TTTGAGGACTAAAGACTTTTTCATGAGG |
| | TAACACCGCCTGC | | AAGTTTCCAT |
| 124 | AACAGTGCCACGCTGAGAGCCAGC | 219 | TAAACGGGTAAAATACGTAATGCCACT |
| | AGCAAATGAAAAAT | | ACGAAGGCACC |
| 125 | CTAAAGCATCACCTTGCTGAACCTC | 220 | AACCTAAAACGAAAGAGGCAAAAGAAT |
| | AAATATCAAACCC | | ACACTAAAACA |
| 126 | TCAATCAATATCTGGTCAGTTGGCA | 221 | CTCATCTTTGACCCCCAGCGATTATAC |
| | AATCAACAGTTGA | | CAAGCGCGAAA |
| 127 | AAGGAATTGAGGAAGGTTATCTAAA | 222 | CAAAGTACAACGGAGATTTGTATCATC |
| | ATATCTTTAGGAG | | GCCTGATAAAT |
| 128 | CACTAACAACTAATAGATTAGAGCC | 223 | TGTGTCGAAATCCGCGACCTGCTCCAT |
| | GTCAATAGATAAT | | GTTACTTAGCC |
| 129 | ACATTTGAGGATTTAGAAGTATTAGA | 224 | GGAACGAGGCGCAGACGGTCAATCAT |
| | CTTTACAAACAA | | AAGGGAACCGAA |
| 130 | TTCGACAACTCGTATTAAATCCTTTG | 225 | CTGACCAACTTTGAAAGAGGACAGATG |
| | CCCGAACGTTAT | | AACGGTGTACA |
| 131 | TAATTTTAAAAGTTTGAGTAACATTA | 226 | GACCAGGCGCATAGGCTGGCTGACCT |
| | TCATTTTGCGGA | | TCATCAAGAGTA |
| 132 | ACAAAGAAACCACCAGAAGGAGCG | 227 | ATCTTGACAAGAACCGGATATTCATTAC |
| | GAATTATCATCATA | | CCAAATCAAC |
| 133 | TTCCTGATTATCAGATGATGGCAATT | 228 | GTAACAAAGCTGCTCATTCAGTGAATA |
| | CATCAATATAAT | | AGGCTTGCCCT |
| 134 | CCTGATTGTTTGGATTATACTTCTGA | 229 | GACGAGAAACACCAGAACGAGTAGTAA |
| | ATAATGGAAGGG | | ATTGGGCTTGA |
| 135 | TTAGAACCTACCATATCAAAATTATT | 230 | GATGGTTTAATTTCAACTTTAATCATTG |
| | TGCACGTAAAAC | | TGAATTACCT |
| 136 | AGAAATAAAGAAATTGCGTAGATTTT | 231 | TATGCGATTTTAAGAACTGGCTCATTAT |
| | CAGGTTTAACGT | | ACCAGTCAGG |
| 137 | CAGATGAATATACAGTAACAGTACC | 232 | ACGTTGGGAAGAAAAATCTACGTTAAT |
| | TTTTACATCGGGA | | AAAACGAACTA |
| 138 | GAAACAATAACGGATTCGCCTGATT | 233 | ACGGAACAACATTATTACAGGTAGAAA |
| | GCTTTGAATACCA | | GATTCATCAGT |
| 139 | AGTTACAAAATCGCGCAGAGGCGAA | 234 | TGAGATTTAGGAATACCACATTCAACTA |
| | TTATTCATTTCAA | | ATGCAGATAC |
| 140 | TTACCTGAGCAAAAGAAGATGATGA | 235 | ATAACGCCAAAAGGAATTACGAGGCAT |
| | AACAAACATCAAG | | AGTAAGAGCAA |
| 141 | AAAACAAAATTAATTACATTTAACAA | 236 | CACTATCATAACCCTCGTTTACCAGAC |
| | TTTCATTTGAAT | | GACGATAAAAA |
| 142 | TACCTTTTTTAATGGAAACAGTACAT | 237 | CCAAAATAGCGAGAGGCTTTTGCAAAA |
| | AAATCAATATAT | | GAAGTTTTGCC |
| 143 | GTGAGTGAATAACCTTGCTTCTGTA | 238 | AGAGGGGGTAATAGTAAAATGTTTAGA |
| | AATCGTCGCTATT | | CTGGATAGCGT |
| 144 | AATTAATTTTCCCTTAGAATCCTTGA | 239 | CCAATACTGCGGAATCGTCATAAATATT |
| | AAACATAGCGAT | | CATTGAATCC |
| 145 | AGCTTAGATTAAGACGCTGAGAAGA | 240 | CCCTCAAATGCTTTAAACAGTTCAGAAA |
| | GTCAATAGTGAAT | | ACGAGAATGA |
| 146 | TTATCAAAATCATAGGTCTGAGAGA | 241 | CCATAAATCAAAAATCAGGTCTTTACCC |
| | CTACCTTTTTAAC | | TGACTATTAT |
| 147 | CTCCGGCTTAGGTTGGGTTATATAA | 242 | AGTCAGAAGCAAAGCGGATTGCATCAA |
| | CTATATGTAAATG | | AAAGATTAAGA |
| 148 | CTGATGCAAATCCAATCGCAAGACA | 243 | GGAAGCCCGAAAGACTTCAAATATCGC |
| | AAGAACGCGAGAA | | GTTTTAATTCG |
| 149 | AACTTTTTCAAATATATTTTAGTTAAT | 244 | AGCTTCAAAGCGAACCAGACCGGAAG |
| | TTCATCTTCTG | | CAAACTCCAACA |
| 150 | ACCTAAATTTAATGGTTTGAAATACC | 245 | GGTCAGGATTAGAGAGTACCTTTAATT |
| | GACCGTGTGATA | | GCTCCTTTTGA |
| 151 | AATAAGGCGTTAAATAAGAATAAACA | 246 | TAAGAGGTCATTTTTGCGGATGGCTTA |
| | CCGGAATCATAA | | GAGCTTAATTG |
| 152 | TTACTAGAAAAAGCCTGTTTAGTATC | 247 | CTGAATATAATGCTGTAGCTCAACATGT |
| | ATATGCGTTATA | | TTTAAATATG |
| 153 | CAAATTCTTACCAGTATAAAGCCAAC | 248 | CAACTAAAGTACGGTGTCTGGAAGTTT |
| | GCTCAACAGTAG | | CATTCCATATA |
| 154 | GGCTTAATTGAGAATCGCCATATTTA | 249 | ACAGTTGATTCCCAATTCTGCGAACGA |
| | ACAACGCCAACA | | GTAGATTTAGT |
| 155 | TGTAATTTAGGCAGAGGCATTTTCG | 250 | TTGACCATTAGATACATTTCGCAAATGG |
| | AGCCAGTAATAAG | | TCAATAACCT |
| 156 | AGAATATAAAGTACCGACAAAAGGT | 251 | GTTTAGCTATATTTTCATTTGGGGCGCG |
| | AAAGTAATTCTGT | | AGCTGAAAAG |
| 157 | CCAGACGACGACAATAAACAACATG | 252 | GTGGCATCAATTCTACTAATAGTAGTAG |
| | TTCAGCTAATGCA | | CATTAACATC |
| 158 | GAACGCGCCTGTTTATCAACAATAG | 253 | CAATAAATCATACAGGCAAGGCAAAGA |
| | ATAAGTCCTGAAC | | ATTAGCAAAAT |
| 159 | AAGAAAAATAATATCCCATCCTAATT | 254 | TAAGCAATAAAGCCTCAGAGCATAAAG |
| | TACGAGCATGTA | | CTAAATCGGTT |
| 160 | GAAACCAATCAATAATCGGCTGTCT | 255 | GTACCAAAAACATTATGACCCTGTAATA |
| | TTCCTTATCATTC | | CTTTTGCGGG |
| 161 | CAAGAACGGGTATTAAACCAAGTAC | 256 | AGAAGCCTTTATTTCAACGCAAGGATA |
| | CGCACTCATCGAG | | AAAATTTTTAG |
| 162 | AACAAGCAAGCCGTTTTTATTTTCAT | 257 | AACCCTCATATATTTTAAATGCAATGCC |
| | CGTAGGAATCAT | | TGAGTAATGT |
| 163 | TACCGCGCCCAATAGCAAGCAAATC | 258 | GTAGGTAAAGATTCAAAAGGGTGAGAA |
| | AGATATAGAAGGC | | AGGCCGGAGAC |
| 164 | TTATCCGGTATTCTAAGAACGCGAG | 259 | AGTCAAATCACCATCAATATGATATTCA |
| | GCGTTTTAGCGAA | | ACCGTTCTAG |
| 165 | CCTCCCGACTTGCGGGAGGTTTTGA | 260 | CTGATAAATTAATGCCGGAGAGGGTAG |
| | AGCCTTAAATCAA | | CTATTTTTGAG |
| 166 | GATTAGTTGCTATTTTGCACCCAGCT | 261 | AGATCTACAAAGGCTATCAGGTCATTG |
| | ACAATTTTATCC | | CCTGAGAGTCT |
| 167 | TGAATCTTACCAACGCTAACGAGCG | 262 | GGAGCAAACAAGAGAATCGATGAACG |
| | TCTTTCCAGAGCC | | GTAATCGTAAAA |
| 168 | TAATTTGCCAGTTACAAAATAAACAG | 263 | CTAGCATGTCAATCATATGTACCCCGG |
| | CCATATTATTTA | | TTGATAATCAG |
| 169 | TCCCAATCCAAATAAGAAACGATTTT | 264 | AAAAGCCCCAAAAACAGGAAGATTGTA |
| | TTGTTTAACGTC | | TAAGCAAATAT |
| 170 | AAAAATGAAAATAGCAGCCTTTACA | 265 | TTAAATTGTAAACGTTAATATTTTGTTAA |
| | GAGAGAATAACAT | | AATTCGCAT |
| 171 | AAAAACAGGGAAGCGCATTAGACGG | 266 | TAAATTTTTGTTAAATCAGCTCATTTTTT |
| | GAGAATTAACTGA | | AACCAATAG |
| 172 | ACACCCTGAACAAAGTCAGAGGGTA | 267 | GAACGCCATCAAAAATAATTCGCGTCT |
| | ATTGAGCGCTAAT | | GGCCTTCCTGT |
| 173 | ATCAGAGAGATAACCCACAAGAATT | 268 | AGCCAGCTTTCATCAACATTAAATGTGA |
| | GAGTTAAGCCCAA | | GCGAGTAACA |
| 174 | TAATAAGAGCAAGAAACAATGAAATA | 269 | ACCCGTCGGATTCTCCGTGGGAACAAA |
| | GCAATAGCTATC | | CGGCGGATTGA |
| 175 | TTACCGAAGCCCTTTTTAAGAAAAGT | 270 | CCGTAATGGGATAGGTCACGTTGGTGT |
| | AAGCAGATAGCC | | AGATGGGCGCA |
| 176 | GAACAAAGTTACCAGAAGGAAACCG | 271 | TCGTAACCGTGCATCTGCCAGTTTGAG |
| | AGGAAACGCAATA | | GGGACGACGAC |
| 177 | ATAACGGAATACCCAAAAGAACTGG | 272 | AGTATCGGCCTCAGGAAGATCGCACTC |
| | CATGATTAAGACT | | CAGCCAGCTTT |
| 178 | CCTTATTACGCAGTATGTTAGCAAAC | 273 | CCGGCACCGCTTCTGGTGCCGGAAAC |
| | GTAGAAAATACA | | CAGGCAAAGCGC |
| 179 | TACATAAAGGTGGCAACATATAAAA | 274 | CATTCGCCATTCAGGCTGCGCAACTGT |
| | GAAACGCAAAGAC | | TGGGAAGGGCG |
| 180 | ACCACGGAATAAGTTTATTTTGTCAC | 275 | ATCGGTGCGGGCCTCTTCGCTATTACG |
| | AATCAATAGAAA | | CCAGCTGGCGA |
| 181 | ATTCATATGGTTTACCAGCGCCAAA | 276 | AAGGGGGATGTGCTGCAAGGCGATTA |
| | GACAAAAGGGCGA | | AGTTGGGTAACG |
| 182 | CATTCAACCGATTGAGGGAGGGAAG | 277 | CCAGGGTTTTCCCAGTCACGACGTTGT |
| | GTAAATATTGACG | | AAAACGACGGC |
| 183 | GAAATTATTCATTAAAGGTGAATTAT | 278 | CAGTGCCAAGCTTGCATGCCTGCAGGT |
| | CACCGTCACCGA | | CGACTCTAGAGGATCTTTT |

### Example 6

### Discrimination of Control SARS-COV-2 RNA Virus Variants

DNA carriers were designed to detect the presence of wild type SARS-COV-2 and the presence of two single nucleotide and single amino acid variants, N501T and N501S. DNA carriers for SARS-COV-2 N501 RNA virus variants was prepared as described herein. The sequences of SARS-COV-2 N501 RNA, capture oligos, biotinylated strand, and cutting oligos for RNase H cutting are provided in Table 23.

### Programmable RNase H Cutting of SARS-COV-2 RNA Controls

For nanopore sensing, SARS-COV-2 RNA (S:N501 in Table 23) controls were used for the detection with DNA carriers. Firstly, cutting oligos were mixed with a SARS-COV-2 N501 RNA in a ratio 1:1:1 and the mixture was heated to 70° C. for 5 minutes. RNase H (5,000 units/ml, NEB) was added, mixed, and heated for 20 minutes at 37° C. to allow the enzyme to cut RNA in the DNA: RNA hybrid to effectively releases target RNA probes. RNase H was thermally inactivated by incubation at 65° C. for 10 min.

### Nanopore Readout of DNA Carrier

DNA carrier was mixed with cut SARS-COV-2 RNA (S:N501 in Table 23, protocol see above) at ten times excess in 10 mM MgCl₂ and 100 mM NaCl. The mixture (5 μL) was incubated at room temperature (^{˜}10 min) until prepared for nanopore measurement.

### Results

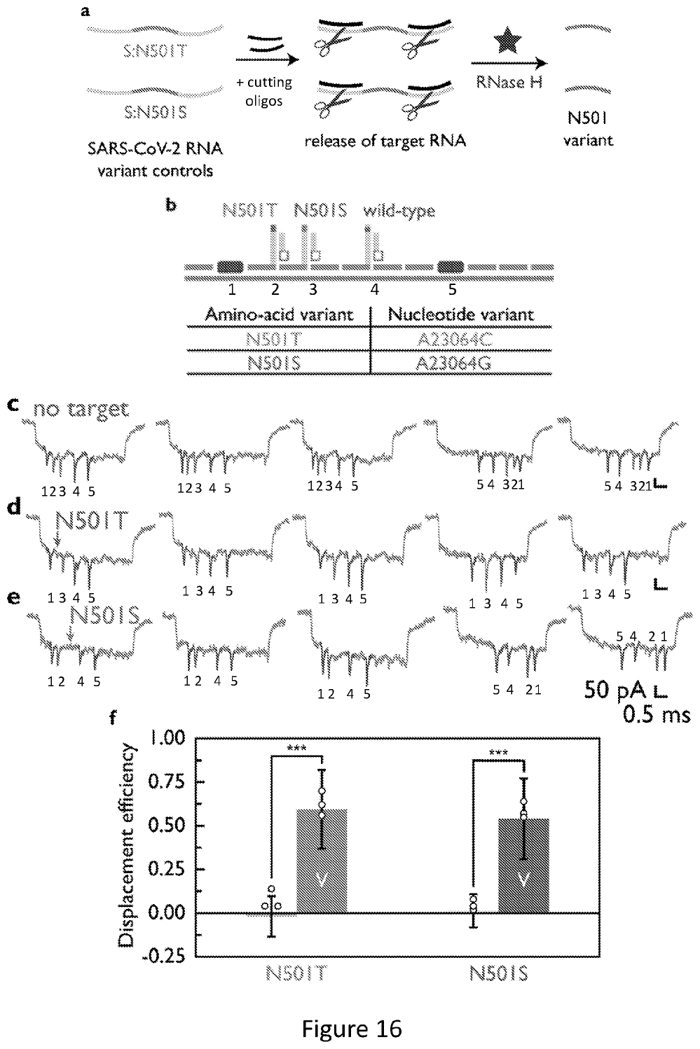

Nanopore readouts confirmed that the SARS-COV-2 capture oligos were specific to the variants they were designed to identify. The N501T target probe displaced the signalling oligo annealed to the N501T capture oligo, and not the N501S or the wild-type capture oligo as evidenced by the presence of the N501S and wild-type signals in the presence of N501T target probe (FIG. 16(d)). The same was true for N501S target probe which interacted only with the N501S capture oligo (FIG. 16(e)). This result is confirmed by FIG. 16(f) which demonstrates that the variant probes exhibited a significantly higher displacement efficiency than the wild-type probe for their respective capture oligos.

These results further confirm that the method of the invention can be used to discriminate between RNA probes containing single-nucleotide variants (FIG. 16).

TABLE 23
SARS-CoV-2 RNA sequences and cutting oligos, capture sites and biotinylated strands.

| SEQ ID | | | | |
|---|---|---|---|---|
| NO | Strand name | Description | | Sequence (5′→3′) |
| 279 | S:N501_RNA | Wild-type oligo | RNA | ATCATATGGTTTCCAACCCA |
| | | | | CTTATGGTGTTGGTTACCAA |
| | | | | CCATACAGAGTAGTAGTACT |
| 280 | S:N501T_RNA | A23064C | RNA | ATCATATGGTTTCCAACCCA |
| | | | | CTTCTGGTGTTGGTTACCAA |
| | | | | CCATACAGAGTAGTAGTACT |
| 281 | S:N501S_RNA | A23064G | RNA | ATCATATGGTTTCCAACCCA |
| | | | | CTTGTGGTGTTGGTTACCAA |
| | | | | CCATACAGAGTAGTAGTACT |
| 282 | Cutting oligo | Downstream | DNA | TGGGTTGGAAACCATATGAT |
| | S:N501_a | cutting oligo | | |
| 283 | Cutting oligo | Upstream | DNA | AGTACTACTACTCTGTATGG |
| | S:N501_b | cutting oligo | | |
| 284 | CS42:N501T | Carrier | DNA | TTCGACAACTCGTATTAAATCCTTTGCCC |
| | | overhang | | GAACGTTAT TTTTT |
| 285 | cS55:N501S | Carrier | DNA | GTGAGTGAATAACCTTGCTTCTGTAAAT |
| | | overhang | | CGTCGCTATT TTTTT |
| 286 | cSUK_81 | Carrier | DNA | TCCCAATCCAAATAAGAAACGATTTTTT |
| | | overhang | | GTTTAACGTC TTTTT |
| | | | | TTGGTAACCAACACCAAAAG |
| 287 | bS:N501 | Signalling oligo | DNA | GTGTTGGTTACCAA/3-BIOTIN/ |

1. A method for detecting the presence or absence of a target nucleic acid in a sample, the method comprising the steps of:
(a) contacting the sample with a cutting reagent for excising a target probe from the target nucleic acid to provide an excise mixture;
(b) contacting the excise mixture with a nucleic acid carrier including a capture oligonucleotide that is complementary to the target probe; and
(c) detecting binding of the target probe to the capture oligonucleotide;
wherein binding of the target probe to the capture oligonucleotide indicates presence of the target nucleic acid in the sample, and the absence of binding of the target probe to the capture oligonucleotide indicates absence of the target nucleic acid in the sample.

2. The method of claim 1, wherein the cutting reagent includes: (a) cutting oligonucleotides which are complementary to target nucleic acid sequences immediately upstream and immediately downstream of the target probe, and (b) an enzyme suitable for cutting the target nucleic acid at sites hybridized to cutting oligonucleotides.

3. The method of claim 1, wherein the target nucleic acid is RNA, optionally wherein the target nucleic acid is selected from single-stranded RNA, double-stranded RNA, mRNA, miRNA, and non-coding RNA.

4. The method of claim 2, wherein the target nucleic acid is single-stranded RNA and the cutting oligonucleotides comprised single-stranded DNA.

5. The method of claim 4, wherein the enzyme suitable for cutting the target nucleic acid at sites hybridized to cutting oligonucleotides is ribonuclease H (RNase H).

6. The method of claim 1, wherein the nucleic acid carrier is a single stranded DNA (ssDNA) carrier.

7. The method of claim 1, in which the nucleic acid carrier comprises one or more reference labels that allow the identity of the nucleic acid carrier, the location of the capture oligonucleotides, and/or the identity of the capture oligonucleotides to be determined.

8. The method of claim 1, wherein the nucleic acid carrier comprises more than one capture oligonucleotide complementary to different target probes, optionally wherein the different target probes are derived from different target nucleic acids.

9. The method of claim 1, wherein the capture oligonucleotide binds to a signaling oligonucleotide in the absence of target probe.

10. The method of claim 9, in which the signaling oligonucleotide is displaced from the capture oligonucleotide in the presence of target probe.

11. The method of claim 10, wherein the capture oligonucleotide comprises an overhang that is complementary to the target probe but is not complementary to the signaling oligonucleotide, and wherein in the presence of target probe, the target probe binds to the overhang and displaces the signaling oligonucleotide from the capture oligonucleotide.

12. The method of claim 9, wherein detecting binding of the target probe to the capture oligonucleotide includes detecting binding of the capture oligonucleotide to the signaling oligonucleotide.

13. The method of claim 9, wherein the signaling oligonucleotide comprises a structural, chemical and/or fluorescent label.

14. The method of claim 13, in which the signaling oligonucleotide comprises a ligand label, and optionally in which the method further comprises contacting the nucleic acid carrier with a receptor that interacts with the ligand.

15. The method of claim 14, wherein the ligand is biotin and the receptor is avidin, neutravidin, traptavidin or streptavidin, and wherein detecting binding of the capture oligonucleotide to the signaling oligonucleotide comprises detecting the presence of biotin, avidin, neutravidin, traptavidin, streptavidin and/or biotin/avidin, biotin/neutravidin, biotin/traptavidin or biotin/streptavidin complexes.

16. The method of claim 14, in which the ligand is an antigen and the receptor is an antibody, and in which detecting binding of the capture oligonucleotide to the signaling oligonucleotide comprises detecting the presence of antigen and/or antigen/antibody complexes.

17. The method of claim 9, wherein the capture oligonucleotide comprised a fluorescent label and the signaling oligonucleotide comprised a quencher and wherein detecting binding of the target probe to the capture oligonucleotide comprised detecting the presence or absence fluorescence.

18. The method of claim 1, wherein binding of the target probe to the capture oligonucleotide is detected using nanopore-based detection methods.

19. The method of claim 1, wherein binding of the target probe to the capture oligonucleotide is detected by spectroscopic-based detection methods.

20. The method of claim 1, wherein the method further comprised quantifying the level of target nucleic acid in the sample by quantifying the level of binding of the target probe to the capture oligonucleotide and/or by quantifying the level of binding of the signaling oligonucleotide to the capture oligonucleotide.

21. The method of claim 1, wherein the method comprises detecting the presence or absence of more than one target nucleic acid in the sample.

22. The method of claim 1, wherein the method comprises excising more than one target probe from the target nucleic acid, optionally from more than one target nucleic acid.

23. The method of claim 1, wherein the target probe has a GC content of 40-60%.

24. The method of claim 1, wherein the target probe comprises a terminal region that has a GC content of 40-60%, optionally wherein the terminal region of the target probe is 1 nt, 2 nt, 3 nt, 4 nt, 5 nt, 6 nt, 7 nt, 8 nt, 9 nt, 10 nt, 15 nt, 20 nt, 30 nt, 40 nt, or 50 nt starting from the 3′ and/or the 5′ end of the target probe.

25. The method of claim 1, wherein the target probe has less than 80% sequence identity to other sequences that may be present in the sample, such as other regions of the target nucleic acid.

26. The method of claim 1, wherein the target probe is located in an unhybridized region of the target nucleic acid.

27. The method of claim 1, wherein the target nucleic acid is derived from a virus, optionally wherein the virus is selected from a coronavirus, Influenza virus, Zika virus, Ebola virus, Dengue virus, Hantavirus, Nairovirus, Orthobunyavirus, Phlebovirus, Flavivirus, and Alphavirus.

28. The method of claim 27, wherein the target nucleic acid is a coronavirus genome, optionally the SARS-COV-2 genome.

29. The method of claim 1, in which the target nucleic acid is derived from a microorganism, optionally in which the target nucleic acid is derived from a bacteria or a fungus.

30. The method of claim 1, in which the target nucleic acid is derived from a pathogen, optionally in which the pathogen is a viral pathogen, bacterial pathogen or a fungal pathogen.

31. The method of claim 1, in which the target nucleic acid is an RNA transcript.

32. The method of claim 1, wherein the target nucleic acid is a therapeutic nucleic acid, optionally wherein the therapeutic nucleic acid is selected from siRNA, shRNA, miRNA, RNA aptamer, DNA aptamer, mRNA, splice-switching oligonucleotides, antisense oligonucleotides, RNA decoys and peptide nucleic acids.

33. The method of claim 1, wherein the target nucleic acid is a genetic biomarker, optionally wherein the target nucleic acid is selected from a gene, an RNA transcript or a region thereof.

34. The method of claim 33, wherein the genetic biomarker is associated with a disease or condition, optionally wherein the disease or condition is cancer or an increased risk thereof, or a hereditary disease or condition.

35. The method of claim 1, wherein the target nucleic acid comprises a single nucleotide variant when compared to a reference nucleic acid.

36. The method of claim 1, wherein the sample is obtained from a subject that has been treated with a therapeutic.

37. The method of claim 36, wherein the method includes comparing the level of target nucleic acid in the sample to the level present in a sample from a subject who has not been treated with the therapeutic.

38. The method of claim 36, wherein the target nucleic acid is an RNA transcript.

39. The method of claim 1, in which the sample is obtained from a subject, optionally in which the subject is a human.

40. The method of claim 39, in which the sample is selected from blood, serum, plasma, saliva, sputum, urine, faeces, cerebrospinal fluid, a lung tissue sample, a bronchoalveolar lavage sample, a nose and/or throat swab sample, or a biopsy sample.
